SEQUENCE LISTING (64 sequences)

SEQ ID NO: 1 - protein, 61 aa, synthetic construct
  source 1..61: note = CD86
KWKKKKRPRNSYKCGTNTMEREESEQTKKREKIHIPERSDEAQRVFKSSKTSSCDKSDTCF

SEQ ID NO: 2 - protein, 36 aa, synthetic construct
  source 1..36: note = LOX1
CWWPSYLFQLGKAKKGNSKEDPQDKVTQIKLDDFTM

SEQ ID NO: 3 - protein, 59 aa, synthetic construct
  source 1..59: note = 2B4
WRRKRKEKQSETSPKEFLTIYEDVKDLKTRRNHEQEQTFPGGGSTIYSMIQSQSSAPTS

SEQ ID NO: 4 - protein, 44 aa, synthetic construct
  source 1..44: note = DECTIN-1
RWPPSAACSGKESVVAIRTNSQSDFHLQTYGDEDLNELDPHYEM

SEQ ID NO: 5 - protein, 20 aa, synthetic construct
  source 1..20: note = DECTIN-2
LRLSLWGRKETSQPQQEQMM

SEQ ID NO: 6 - protein, 48 aa, synthetic construct
  source 1..48: note = TIM1
KKYFFKKEVQQLSVSFSSLQIKALQNAVEKEVQAEDNIYIENSLYATD

SEQ ID NO: 7 - protein, 62 aa, synthetic construct
  source 1..62: note = CD40
KKVAKKPTNKAPHPKQEPQEINFPDDLPGSNTAAPVQETLHGCQPVTQEDGKESRISVQERQ

SEQ ID NO: 8 - protein, 24 aa, synthetic construct
  source 1..24: note = CD11b
KLGFFKRQYKDMMSEGGPPGAEPQ

SEQ ID NO: 9 - protein, 434 aa, synthetic construct
  source 1..434: note = CSF1R
KYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 10 - protein, 54 aa, synthetic construct
  source 1..54: note = CSF2R
KRFLRIQRLFPPVPQIKDKLNDNHEVEDEIIWEEFTPEEGKGYREEVLTVKEIT

SEQ ID NO: 11 - protein, 186 aa, synthetic construct
  source 1..186: note = CSF3R
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 12 - protein, 213 aa, synthetic construct
  source 1..213: note = IL1R
KIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 13 - protein, 185 aa, synthetic construct
  source 1..185: note = TLR1
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 14 - protein, 175 aa, synthetic construct
  source 1..175: note = TLR2
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 15 - protein, 176 aa, synthetic construct
  source 1..176: note = TLR2
CHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 16 - protein, 180 aa, synthetic construct
  source 1..180: note = TLR3
FEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 17 - protein, 187 aa, synthetic construct
  source 1..187: note = TLR4
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 18 - protein, 198 aa, synthetic construct
  source 1..198: note = TLR5
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 19 - protein, 189 aa, synthetic construct
  source 1..189: note = TLR6
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 20 - protein, 189 aa, synthetic construct
  source 1..189: note = TLR7
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 21 - protein, 194 aa, synthetic construct
  source 1..194: note = TLR8
AHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 22 - protein, 388 aa, synthetic construct
  source 1..388: note = SCARF1
CCCWAPRSDLKDRPARDGATVSRMKLQVWGTLTSLGSTLPCRSLSSHKLPWVTVSHHDPEVPFNHSFIEPPSAGWATDDSFSSDPESGEADEVPAYCVPPQEGMVPVAQAGSSEASLAAGAFPPPEDASTPFAIPRTSSLARAKRPSVSFAEGTKFAPQSRRSSGELSSPLRKPKRLSRGAQSGPEGREAEESTGPEEAEAPESFPAAASPGDSATGHRRPPLGGRTVAEHVEAIEGSVQESSGPVTTIYMLAGKPRGSEGPVRSVFRHFGSFQKGQAEAKVKRAIPKPPRQALNRKKGSPGLASGSVGQSPNSAPKAGLPGATGPMAVRPEEAVRGLGAGTESSRRAQEPVSGCGSPEQDPQKQAEEERQEEPEYENVVPISRPPEP

SEQ ID NO: 23 - protein, 409 aa, synthetic construct
  source 1..409: note = SCARF2
CCCACRGKDPTRRPRPRRELSLGRKKAPHRLCGRFSRISMKLPRIPLRRQKLPKVVVAHHDLDNTLNCSFLEPPSGLEQPSPSWSSRASFSSFDTTDEGPVYCVPHEEAPAESRDPEVPTVPAEAPAPSPVPLTTPASAEEAIPLPASSDSERSASSVEGPGGALYARVARREARPARARGEIGGLSLSPSPERRKPPPPDPATKPKVSWIHGKHSAAAAGRAPSPPPPGSEAAPSPSKRKRTPSDKSAHTVEHGSPRTRDPTPRPPGLPEEATALAAPSPPRARARGRGPGLLEPTDAGGPPRSAPEAASMLAAELRGKTRSLGRAEVALGAQGPREKPAPPQKAKRSVPPASPARAPPATETPGPEKAATDLPAPETPRKKTPIQKPPRKKSREAAGELGRAGAPTL

SEQ ID NO: 24 - protein, 37 aa, synthetic construct
  source 1..37: note = DC-SIGN
CGALSKYGRTQRFGLGRLQEEELLGLQQLRPEKSDSM

SEQ ID NO: 25 - protein, 28 aa, synthetic construct
  source 1..28: note = CXCL16
CKRRRGQSPQSSPDLPVHYIPVAPDSNT

SEQ ID NO: 26 - protein, 51 aa, synthetic construct
  source 1..51: note = CD93
GLLVYRKRRAKREEKKEKKPQNAADSYSWVPERAESRAMENQYSPTPGTDC

SEQ ID NO: 27 - protein, 19 aa, synthetic construct
  source 1..19: note = LIMPII
GQGSMDEGTADERAPLIRT

SEQ ID NO: 28 - protein, 52 aa, synthetic construct
  source 1..52: note = MELLEC
LAVPRWTSSPARHETRRPEPHRTTASGQSHLSMTTDGDDDLMDRTSSYKAQM

SEQ ID NO: 29 - protein, 35 aa, synthetic construct
  source 1..35: note = DNGR1
LCCAGSCKNSSLCKQYTDPAPSDWQLSTYIEEEHM

SEQ ID NO: 30 - protein, 43 aa, synthetic construct
  source 1..43: note = TIM4
LRGKLMETYCSQKHTRLDYIGDSKNVLNDVQHGREDEDGLFTL

SEQ ID NO: 31 - protein, 84 aa, synthetic construct
  source 1..84: note = FCER1G
LRKRRDSLSLSTQRTQGPAESARNLEYVSVSPTNNTVYASVTHSNRETEIWTPRENDTITIYSTINHSKESKPTFSRATALDNV

SEQ ID NO: 32 - protein, 571 aa, synthetic construct
  source 1..571: note = VEGFR2
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 33 - protein, 69 aa, synthetic construct
  source 1..69: note = IFNgR2
LVLKYRGLIKYWFHTPPSIPLQIEEYLKDPTQPILEALDKDSSPKDDVWDSVSIISFPEKEQEDVLQTL

SEQ ID NO: 34 - protein, 33 aa, synthetic construct
  source 1..33: note = CLEC2
MVRWWSSSASGVSILAPKRTKINLTIYGDEDQM

SEQ ID NO: 35 - protein, 43 aa, synthetic construct
  source 1..43: note = MARCO
NVGNRRKPKPVNIEFPEMAIQHFAAQQTESLLEDEKLIKKNRM

SEQ ID NO: 36 - protein, 31 aa, synthetic construct
  source 1..31: note = DEC-205
QRHRLHLAGFSSVRYAQGVNEDEIMLPSFHD

SEQ ID NO: 37 - protein, 41 aa, synthetic construct
  source 1..41: note = RAGE
QRRQRRGEERKAPENQEEEEERAELNQSEEPEAGESSTGGP

SEQ ID NO: 38 - protein, 71 aa, synthetic construct
  source 1..71: note = STAB1
RARGKPMGFGFSAFQAEDDADDDFSPWQEGTNPTLVSVPNPVFGSDTFCEPFDDSLLEEDFPDTQRILTVK

SEQ ID NO: 39 - protein, 41 aa, synthetic construct
  source 1..41: note = CD28
RSKRSRLLHSDYMNMTPRRPGPTRKHYQPYAPPRDFAAYRS

SEQ ID NO: 40 - protein, 43 aa, synthetic construct
  source 1..43: note = LANGERIN
RVTPTKGPVLSPGSKPPPERPWLSINQKDVTFHADPAEKEVTM

SEQ ID NO: 41 - protein, 11 aa, synthetic construct
  source 1..11: note = CD36
SYCACRSKTIK

SEQ ID NO: 42 - protein, 26 aa, synthetic construct
  source 1..26: note = CD80
TTYCFAPRCRERRRNERLRRESVRPV

SEQ ID NO: 43 - protein, 25 aa, synthetic construct
  source 1..25: note = CD80
TYCFAPRCRERRRNERLRRESVRPV

SEQ ID NO: 44 - protein, 88 aa, synthetic construct
  source 1..88: note = SLAMF7
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 45 - protein, 52 aa, synthetic construct
  source 1..52: note = DAP12
YFLGRLVPRGRGAAEAATRKQRITETESPYQELQGQRSDVYSDLNTQRPYYK

SEQ ID NO: 46 - protein, 46 aa, synthetic construct
  source 1..46: note = MRC1/CD206
YKKRRVHLPQEGAFENTLYFNSQSSPGTSDMKDLVGNIEQNEHSVI

SEQ ID NO: 47 - protein, 262 aa, synthetic construct
  source 1..262: note = MEGF10
YRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 48 - protein, 72 aa, synthetic construct
  source 1..72: note = STABILIN2
YSYFRINRRTIGFQHFESEEDINVAALGKQQPENISNPLYESTTSAPPEPSYDPFTDSEERQLEGNDPLRTL

SEQ ID NO: 49 - protein, 36 aa, synthetic construct
  source 1..36: note = BAI-1
YWSMRLQARGGPSPLKSNSDSARLPISSGSTSSSRI

SEQ ID NO: 50 - protein, 113 aa, synthetic construct
  source 1..113: note = CD3sigma
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 51 - protein, 55 aa, synthetic construct
  source 1..55: note = CD3epsilon
KNRKAKAKPVTRGAGAGGRQRGQNKERPPPVPNPDYEPIRKGQRDLYSGLNQRRI

SEQ ID NO: 52 - protein, 45 aa, synthetic construct
  source 1..45: note = CD3delta
GHETGRLSGAADTQALLRNDQVYQPLRDRDDAQYSHLGGNWARNK

SEQ ID NO: 53 - protein, 61 aa, synthetic construct
  source 1..61: note = FcgammaRI
RKELKRKKKWDLEISLDSGHEKKVISSLQEDRHLEEELKCQEQKEEQLQEGVHRKEPQGAT

SEQ ID NO: 54 - DNA, 276 bp, synthetic construct
  source 1..276: mol_type = other DNA; note = Synthetic promoter-146 (SP146)
ctagcgagggcggaccagaaaaggagaagtaggagccaagatttccaaactctgtggttgccttgccaagatttccaaactctgtggttgccttgcagaaaaggagaagtaggagaagcgacttcctctttccagaagcgacttcctctttccagaggaagagggcggaggctcacaaggcaaccacagagtttggaaatcttggaagcgacttcctctttccagcagaaaaggagaagtaggagaagcgacttcctctttccaggtccgccctcg

SEQ ID NO: 55 - DNA, 266 bp, synthetic construct
  source 1..266: mol_type = other DNA; note = Synthetic promoter-107 (SP107)
ctagcgagggcggacaagcgacttcctctttccagcagaaaaggagaagtaggagccaagatttccaaactctgtggttgccttgaagcgacttcctctttccagaagcgacttcctctttccagcagaaaaggagaagtaggagctcctacttctccttttctgccaagatttccaaactctgtggttgccttgctcctacttctccttttctgcagaaaaggagaagtaggagctggaaagaggaagtcgcttgagggcggacg

SEQ ID NO: 56 - DNA, 176 bp, synthetic construct
  source 1..176: mol_type = other DNA; note = Synthetic promoter-60 (SP60)
ctagcgtccgccctcaggaagagggcggaggctcaccaagatttccaaactctgtggttgccttgccaagatttccaaactctgtggttgccttgccaagatttccaaactctgtggttgccttgtgagcctccgccctcttcctaagcgacttcctctttccaggagggcggacg

SEQ ID NO: 57 - protein, 18 aa, synthetic construct
  source 1..18: note = T2A
EGRGSLLTCGDVEENPGP

SEQ ID NO: 58 - protein, 19 aa, synthetic construct
  source 1..19: note = P2A
ATNFSLLKQAGDVEENPGP

SEQ ID NO: 59 - protein, 20 aa, synthetic construct
  source 1..20: note = E2A
QCTNYALLKLAGDVESNPGP

SEQ ID NO: 60 - protein, 22 aa, synthetic construct
  source 1..22: note = F2A
VKQTLNFDLLKLAGDVESNPGP

SEQ ID NO: 61 - DNA, 1338 bp, synthetic construct
  source 1..1338: mol_type = other DNA
atggcactgccagtgaccgccctgctgctgccactggccctgctgctgcacgcagccagaccccaagtgcaactggtggaatcaggcggcggagtggtgcagcctggcagaagcctgcggctgtcctgcgcggcctctggattcacattcagcagctacgccatgcactgggtcagacaggcccctggaaaaggcctggaatgggtggccgtgatcagctacgacggcaacaacaagtactacgccgacagcgtgaagggaagattcaccatcagccgggacaacagcaagaacaccctgtacctgcagatgaacagcctgcgggccgaggatacagccgtctactattgtgccagagccgttccttggggctccagatactactactacggcatggacgtgtggggccagggcacaactgtgaccgtgtcctccggtggcggcggctccggcggaggcggaagcggaggcggcggctctgctatccagctgacccagagcccatccagcctgtccgccagcgtgggcgatagagtgacaatcacctgtagagccagccagggaatcagcagcgccctggcctggtatcagcaaaagagcggaaaagcccccaagctgctgatcttcgacgcctctagcctggaatctggcgtgcctagcagattcagcggatctggatctggcaccgactttaccctgacaatctctagcctccagccagaagatttcgctacatactactgccagcagttcaacagctatcctctgaccttcggcggcggaaccaaagtggaaatcaagaccacaaccccagcacctagaccacccacaccagcacccaccatcgcctctcagcctctgagcctgagaccagaggcctgtaggcccgcagcaggaggagcagtgcacaccaggggcctggacttcgcctgcgacatctacatctgggcacctctggccggcacctgtggcgtgctgctgctgagcctggtcatcacacgcgtgaagtttagccggtccgccgacgcaccagcatatcagcagggccagaatcagctgtacaacgagctgaatctgggccggagagaggagtacgacgtgctggataagaggcggggccgggaccccgagatgggaggcaagccacagaggagaaagaacccccaggagggcctgtataatgagctgcagaaggacaagatggccgaggcctactctgagatcggcatgaagggagagaggcgccggggcaagggacacgatggcctgtatcagggcctgagcacagccaccaaggacacctacgatgccctgcacatgcaggccctgcctccaagg

SEQ ID NO: 62 - protein, 446 aa, synthetic construct
MALPVTALLLPLALLLHAARPQVQLVESGGGVVQPGRSLRLSCAASGFTFSSYAMHWVRQAPGKGLEWVAVISYDGNNKYYADSVKGRFTISRDNSKNTLYLQMNSLRAEDTAVYYCARAVPWGSRYYYYGMDVWGQGTTVTVSSGGGGSGGGGSGGGGSAIQLTQSPSSLSASVGDRVTITCRASQGISSALAWYQQKSGKAPKLLIFDASSLESGVPSRFSGSGSGTDFTLTISSLQPEDFATYYCQQFNSYPLTFGGGTKVEIKTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 63 - RNA, 20 nt, synthetic construct
  source 1..20: mol_type = other RNA
cgacctctctgatccctgtg

SEQ ID NO: 64 - DNA, 20 bp, Synthetic construct
  source 1..20: mol_type = other DNA
tgacctccctgatccctgtg

(19) United States
(12) Patent Application Publication
(10) Pub. No.: US 2026/0201333 A1
(43) Pub. Date: Jul. 16, 2026

(54) METHODS AND COMPOSITIONS FOR GENERATING MODIFIED PLURIPOTENT CELLS AND DERIVATES THEREOF

(71) Applicant: SHORELINE BIOSCIENCES, INC., San Diego, CA (US)

(72) Inventors: Christine HUH, San Diego, CA (US); Fereshteh PARVIZ, San Diego, CA (US); David RODGERS, San Diego, CA (US); May SUMI, San Diego, CA (US); Matthew THAYER, San Diego, CA (US); Huafeng WANG, San Diego, CA (US)

(21) Appl. No.: 19/135,719

(22) PCT Filed: Dec. 4, 2023
(86) PCT No.: PCT/US2023/082309

§ 371 Date: Jun. 4, 2025

Related U.S. Application Data
(60) Provisional application No. 63/430,312, filed on Dec. 5, 2022.
(60) Provisional application No. 63/516,301, filed on Jul. 28, 2023.
(60) Provisional application No. 63/598,493, filed on Nov. 13, 2023.

(51) Int. Cl. C12N 5/0786; C07K 14/47; C07K 14/705; C12N 5/074

(52) U.S. Cl. CPC C12N 5/0645; C07K 14/4702; C07K 14/70503; C12N 5/0696; C12N 2506/45; C12N 2510/00

(57) ABSTRACT

The present disclosure relates, in part, to methods and compositions for generating modified pluripotent cells, such as modified pluripotent cells comprising a polynucleotide encoding a chimeric antigen receptor (CAR) and genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene, and derivates thereof (e.g., modified myeloid progenitor cells, modified monocytes, and/or modified macrophages).

### CROSS-REFERENCE TO RELATED APPLICATIONS

This application claims priority to U.S. Provisional Application No. 63/430,312 filed Dec. 5, 2022, U.S. Provisional Application No. 63/516,301 filed Jul. 28, 2023, and U.S. Provisional Application No. 63/598,493 filed Nov. 13, 2023, the content of each of which is incorporated by reference in its entirety herein, and to which priority is claimed.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14735-055-228_SEQLISTING.xml”, was created on Nov. 29, 2023 and is 74,472 bytes in size.

### 1. FIELD

The present disclosure relates, in part, to methods and compositions for generating modified pluripotent cells, such as modified pluripotent cells comprising a polynucleotide or a polypeptide encoding a chimeric antigen receptor (CAR) and genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene, and derivates thereof (e.g., modified myeloid progenitor cells, modified monocytes, and/or modified macrophages).

### 2. BACKGROUND

Macrophages expressing a chimeric antigen receptor have recently emerged as a new possibility for treating solid tumors. However, there are significant engineering hurdles that limit the use of macrophage-based cell therapy. In particular, monocyte-derived macrophages do not expand efficiently in vitro and have a short half-life. Further, attempts to modify a population of macrophages or monocytes directly typically results in a heterogenous population of macrophages, which serves as an additional barrier for clinical use.

Thus, there is an unmet need for sources of macrophages and derivates thereof (e.g., monocytes and myeloid progenitor cells) expressing a chimeric antigen receptor that are suitable for clinical use, such as modified pluripotent cells comprising a polynucleotide or a polypeptide encoding a chimeric antigen receptor (CAR) and genetic disruption of one or more genes that improve the activity of the macrophages.

### 3. SUMMARY

In one aspect, provided herein is a modified pluripotent cell comprising a polynucleotide encoding a chimeric antigen receptor (CAR) and genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the pluripotent cell comprises genetic disruption of the SIRPA gene. In some embodiments, the cell is heterozygous for the genetic disruption of the SIRPA gene. In some embodiments, the cell is homozygous for the genetic disruption of the SIRPA gene. In some embodiments, the pluripotent cell comprises genetic disruption of the SIGLEC10 gene. In some embodiments, the cell is heterozygous for the genetic disruption of the SIGLEC10 gene. In some embodiments, the cell is homozygous for the genetic disruption of the SIGLEC10 gene. In some embodiments, the pluripotent cell comprises genetic disruption of the CISH gene. In some embodiments, the cell is heterozygous for the genetic disruption of the CISH gene. In some embodiments, the cell is homozygous for the genetic disruption of the CISH gene. In some embodiments, the CAR comprises a non-lymphoid intracellular signaling domain. In some embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, CD40, and CD80/B7-1. In some embodiments, the modified pluripotent cell is an induced pluripotent stem cell (iPSC). In some embodiments, the iPSC has been reprogrammed from a cell selected from the group consisting of a peripheral blood mononuclear cell (PBMC), CD34⁺ cord blood, a macrophage, a monocyte, and a fibroblast.

In another aspect, provided herein is a method of generating a modified pluripotent cell, comprising: (a) obtaining a pluripotent cell expressing a polypeptide encoding a chimeric antigen receptor (CAR); and (b) genetically disrupting in the pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene. In one embodiment, the method comprises genetically disrupting the SIRPA gene. In one embodiment, the modified pluripotent cell is heterozygous for the genetic disruption of the SIRPA gene. In one embodiment, the modified pluripotent cell is homozygous for the genetic disruption of the SIRPA gene. In one embodiment, the method comprises genetically disrupting the SIGLEC10 gene. In one embodiment, the modified pluripotent cell is heterozygous for the genetic disruption of the SIGLEC10 gene. In one embodiment, the modified pluripotent cell is homozygous for the genetic disruption of the SIGLEC10 gene. In one embodiment, the method comprises genetically disrupting the CISH gene. In one embodiment, the modified pluripotent cell is heterozygous for the genetic disruption of the CISH gene. In one embodiment, the modified pluripotent cell is homozygous for the genetic disruption of the CISH gene. In one embodiment, the CAR comprises a non-lymphoid intracellular signaling domain. In one embodiment, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, CD40, and CD80/B7-1. In one embodiment, the pluripotent cell is an induced pluripotent stem cell (iPSC). In one embodiment, the iPSC has been reprogrammed from a cell selected from the group consisting of a peripheral blood mononuclear cell (PBMC), CD34⁺ cord blood, a macrophage, a monocyte, and a fibroblast.

In another aspect, provided herein is a method of generating a modified pluripotent cell, comprising introducing a polypeptide encoding a chimeric antigen receptor (CAR) into a pluripotent cell comprising genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In one embodiment, the method comprises genetically disrupting the SIRPA gene. In one embodiment, the modified pluripotent cell is heterozygous for the genetic disruption of the SIRPA gene. In one embodiment, the modified pluripotent cell is homozygous for the genetic disruption of the SIRPA gene. In one embodiment, the method comprises genetically disrupting the SIGLEC10 gene. In one embodiment, the modified pluripotent cell is heterozygous for the genetic disruption of the SIGLEC10 gene. In one embodiment, the modified pluripotent cell is homozygous for the genetic disruption of the SIGLEC10 gene. In one embodiment, the method comprises genetically disrupting the CISH gene. In one embodiment, the modified pluripotent cell is heterozygous for the genetic disruption of the CISH gene. In one embodiment, the modified pluripotent cell is homozygous for the genetic disruption of the CISH gene. In one embodiment, the CAR comprises a non-lymphoid intracellular signaling domain. In one embodiment, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, CD40, and CD80/B7-1. In one embodiment, the pluripotent cell is an induced pluripotent stem cell (iPSC). In one embodiment, the iPSC has been reprogrammed from a cell selected from the group consisting of a peripheral blood mononuclear cell (PBMC), CD34⁺ cord blood, a macrophage, a monocyte, and a fibroblast.

In a further aspect, provided herein is a method of generating a modified pluripotent cell, comprising: (a) introducing a polypeptide encoding a chimeric antigen receptor (CAR) into a pluripotent cell; and (b) genetically disrupting in the pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene. In one embodiment, the method comprises genetically disrupting the SIRPA gene. In one embodiment, the modified pluripotent cell is heterozygous for the genetic disruption of the SIRPA gene. In one embodiment, the modified pluripotent cell is homozygous for the genetic disruption of the SIRPA gene. In one embodiment, the method comprises genetically disrupting the SIGLEC10 gene. In one embodiment, the modified pluripotent cell is heterozygous for the genetic disruption of the SIGLEC10 gene. In one embodiment, the modified pluripotent cell is homozygous for the genetic disruption of the SIGLEC10 gene. In one embodiment, the method comprises genetically disrupting the CISH gene. In one embodiment, the modified pluripotent cell is heterozygous for the genetic disruption of the CISH gene. In one embodiment, the modified pluripotent cell is homozygous for the genetic disruption of the CISH gene. In one embodiment, the CAR comprises a non-lymphoid intracellular signaling domain. In one embodiment, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, CD40, and CD80/B7-1. In one embodiment, the pluripotent cell is an induced pluripotent stem cell (iPSC). In one embodiment, the iPSC has been reprogrammed from a cell selected from the group consisting of a peripheral blood mononuclear cell (PBMC), CD34⁺ cord blood, a macrophage, a monocyte, and a fibroblast.

In yet another aspect, provided herein is a method of generating a modified pluripotent cell, comprising: (a) genetically disrupting in a pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene; and (b) introducing a polypeptide encoding a chimeric antigen receptor (CAR) into the pluripotent cell. In one embodiment, the method comprises genetically disrupting the SIRPA gene. In one embodiment, the modified pluripotent cell is heterozygous for the genetic disruption of the SIRPA gene. In one embodiment, the modified pluripotent cell is homozygous for the genetic disruption of the SIRPA gene. In one embodiment, the method comprises genetically disrupting the SIGLEC10 gene. In one embodiment, the modified pluripotent cell is heterozygous for the genetic disruption of the SIGLEC10 gene. In one embodiment, the modified pluripotent cell is homozygous for the genetic disruption of the SIGLEC10 gene. In one embodiment, the method comprises genetically disrupting the CISH gene. In one embodiment, the modified pluripotent cell is heterozygous for the genetic disruption of the CISH gene. In one embodiment, the modified pluripotent cell is homozygous for the genetic disruption of the CISH gene. In one embodiment, the CAR comprises a non-lymphoid intracellular signaling domain. In one embodiment, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, Dectin-1, CD206, Dectin-3, CLEC2, CD40, and CD80/B7-1. In one embodiment, the pluripotent cell is an induced pluripotent stem cell (iPSC). In one embodiment, the iPSC has been reprogrammed from a cell selected from the group consisting of a peripheral blood mononuclear cell (PBMC), CD34⁺ cord blood, a macrophage, a monocyte, and a fibroblast.

In one aspect, provided herein is a homogenous population of modified myeloid progenitor cells comprising a polynucleotide encoding a chimeric antigen receptor (CAR), and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the cells comprise genetic disruption of the SIRPA gene. In some embodiments, the cells are heterozygous for the genetic disruption of the SIRPA gene. In some embodiments, the cells are homozygous for the genetic disruption of the SIRPA gene. In some embodiments, the cells comprise genetic disruption of the SIGLEC10 gene. In some embodiments, the cells are heterozygous for the genetic disruption of the SIGLEC10 gene. In some embodiments, the cells are homozygous for the genetic disruption of the SIGLEC10 gene. In some embodiments, the cells comprise genetic disruption of the CISH gene. In some embodiments, the cells are heterozygous for the genetic disruption of the CISH gene. In some embodiments, the cells are homozygous for the genetic disruption of the CISH gene. In some embodiments, the cells are isogenic. In some embodiments, the CAR comprises a non-lymphoid intracellular signaling domain. In some embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In some embodiments, the cells are derived from induced pluripotent stem cells (iPSCs). In some embodiments, the iPSCs have been reprogrammed from a cell selected from the group consisting of a peripheral blood mononuclear cell (PBMC), CD34⁺ cord blood, a macrophage, a monocyte, and a fibroblast.

In another aspect, provided herein is a method of generating the homogeneous population of modified myeloid progenitor cells provided herein, comprising expanding and differentiating the modified pluripotent cell provided herein under conditions sufficient for cell differentiation into a population of myeloid progenitor cells. In some embodiments, the modified pluripotent cell is an induced pluripotent stem cell (iPSC).

In another aspect, provided herein is a homogenous population of modified monocytes comprising a polynucleotide encoding a chimeric antigen receptor (CAR), and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified monocytes comprise genetic disruption of the SIRPA gene. In some embodiments, the modified monocytes are heterozygous for the genetic disruption of the SIRPA gene. In some embodiments, the modified monocytes are homozygous for the genetic disruption of the SIRPA gene. In some embodiments, the modified monocytes comprise genetic disruption of the SIGLEC10 gene. In some embodiments, the modified monocytes are heterozygous for the genetic disruption of the SIGLEC10 gene. In some embodiments, the modified monocytes are homozygous for the genetic disruption of the SIGLEC10 gene. In some embodiments, the modified monocytes comprise genetic disruption of the CISH gene. In some embodiments, the modified monocytes are homozygous for the genetic disruption of the CISH gene. In some embodiments, the modified monocytes are homozygous for the genetic disruption of the CISH gene. In some embodiments, the modified monocytes are isogenic. In some embodiments, the CAR comprises a non-lymphoid intracellular signaling domain. In some embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In some embodiments, the modified monocytes are derived from induced pluripotent stem cells (iPSCs). In some embodiments, the iPSCs have been reprogrammed from a cell selected from the group consisting of a peripheral blood mononuclear cell (PBMC), CD34⁺ cord blood, a macrophage, a monocyte, and a fibroblast.

In another aspect, provided herein is a method of generating the homogeneous population of monocytes provided herein, comprising expanding and differentiating the modified pluripotent cell provided herein under conditions sufficient for cell differentiation into a population of monocytes. In some embodiments, the modified pluripotent cell is an induced pluripotent stem cell (iPSC). In another aspect, provided herein is a homogenous population of modified macrophages comprising a polynucleotide encoding a chimeric antigen receptor (CAR), and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified macrophages comprise genetic disruption of the SIRPA gene. In some embodiments, the modified macrophages are heterozygous for the genetic disruption of the SIRPA gene. In some embodiments, the modified macrophages are homozygous for the genetic disruption of the SIRPA gene. In some embodiments, the modified macrophages comprise genetic disruption of the SIGLEC10 gene. In some embodiments, the modified macrophages are heterozygous for the genetic disruption of the SIGLEC10 gene. In some embodiments, the modified macrophages are homozygous for the genetic disruption of the SIGLEC10 gene. In some embodiments, the modified macrophages comprise genetic disruption of the CISH gene. In some embodiments, the modified macrophages are heterozygous for the genetic disruption of the CISH gene. In some embodiments, the modified macrophages are homozygous for the genetic disruption of the CISH gene. In some embodiments, the modified macrophages are isogenic. In some embodiments, the CAR comprises a non-lymphoid intracellular signaling domain. In some embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In some embodiments, the modified macrophages are derived from induced pluripotent stem cells (iPSCs). In some embodiments, the iPSCs have been reprogrammed from a cell selected from the group consisting of a peripheral blood mononuclear cell (PBMC), CD34⁺ cord blood, a macrophage, a monocyte, and a fibroblast.

In another aspect, provided herein is a method of generating the homogeneous population of macrophages provided herein comprising expanding and differentiating the modified pluripotent cell provided herein or the modified monocytes provided herein under conditions sufficient for cell differentiation into a population of macrophages. In some embodiments, the modified pluripotent cell is an induced pluripotent stem cell (iPSC).

In another aspect, provided herein is a homogenous population of modified CD11b⁺CD45⁺ cells comprising a polynucleotide encoding a chimeric antigen receptor (CAR), and genetic disruption of: (a) a signal regulatory protein alpha (SIRPA) gene; (b) a cytokine inducible SH2 containing protein (CISH) gene; and/or (c) sialic acid-binding Ig-like lectin 10 (SIGLEC10) gene. In certain embodiments, the CD11b⁺CD45⁺ cells comprise genetic disruption of the SIRPA gene. In certain embodiments, the CD11b⁺CD45⁺ cells are heterozygous for the genetic disruption of the SIRPA gene. In certain embodiments, the CD11b⁺CD45⁺ cells are homozygous for the genetic disruption of the SIRPA gene. In certain embodiments, the CD11b⁺CD45⁺ cells comprise genetic disruption of the SIGLEC10 gene. In certain embodiments, the CD11b⁺CD45⁺ cells are heterozygous for the genetic disruption of the SIGLEC10 gene. In certain embodiments, the CD11b⁺CD45⁺ cells are homozygous for the genetic disruption of the SIGLEC10 gene. In certain embodiments, the CD11b⁺CD45⁺ cells comprise genetic disruption of the CISH gene. In certain embodiments, the CD11b⁺CD45⁺ cells are heterozygous for the genetic disruption of the CISH gene. In certain embodiments, the CD11b⁺CD45⁺ cells are homozygous for the genetic disruption of the CISH gene. In certain embodiments, the CD11b⁺CD45⁺ cells are isogenic. In certain embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, the CD11b⁺CD45⁺ cells are derived from induced pluripotent stem cells (iPSCs). In certain embodiments, the iPSCs have been reprogrammed from a cell selected from the group consisting of a peripheral blood mononuclear cell (PBMC), CD34⁺ cord blood, a macrophage, a monocyte, and a fibroblast. In certain embodiments, the modified CD11b⁺CD45⁺ cell is a CD11b⁺CD45⁺CD14⁻ cell or a CD11b⁺CD45⁺CD14⁺ cell.

In another aspect, provided herein is a method of generating the homogeneous population of modified CD11b⁺CD45⁺ cells provided herein, comprising expanding and differentiating the modified pluripotent cell provided herein under conditions sufficient for cell differentiation into a population of CD11b⁺CD45⁺ cells. In certain embodiments, the modified pluripotent cell is an induced pluripotent stem cell (iPSC).

As provided herein, the modified cells of the present disclosure (e.g., a modified cell described in Section 5.1) or population of modified cells of the present disclosure are suitable for use as a cell-based therapy or therapies, such as in a method of treating cancer, for example a head and neck cancer, breast cancer, prostate cancer, or B cell cancer.

### 4. BRIEF DESCRIPTION OF THE DRAWINGS

FIG. 1 illustrates a western blot confirming SIRPA knockout in monocytes 17 days and 20 days after differentiation of SIRPA KO iPSCs. Monocytes derived from TC-1133 iPSCs that expressed SIRPA were wild-type control cells. Vinculin served as the housekeeping gene control.

FIG. 2-FIG. 3 illustrate flow cytometry data for iPSC derived SIRPA KO monocytes at Day 17 (FIG. 2) and Day 20 (FIG. 3) relative to wild-type control monocytes derived from TC-1133 iPSCs that expressed SIRPA. Live singlets were gated on CD45⁺ and then CD11b⁺CD14⁺ to confirm monocyte purity.

FIG. 4A-FIG. 4C illustrate flow cytometry data for SIRPα expression using an antibody that detects SIRPα surface expression (FIG. 4B) or intracellular expression (FIG. 4C) in iPSC-derived SIRPA KO macrophages obtained from D17 or D20 iPSC-derived monocytes, relative to wild-type control monocytes derived from TC-1133 iPSCs that expressed SIRPA. Isotype controls were used as a negative control to determine background caused by nonspecific antibody binding (FIG. 4A).

FIG. 5A and FIG. 5B illustrate that SIRPA KO monocytes derived from SIRPA KO iPSCs had increased killing on Raji cells relative to wild-type control monocytes derived from TC-1133 iPSCs that expressed SIRPA, with the addition of 0.3 μg/mL and 0.5 μg/mL rituximab. The amount of target cell survival was measured from 0 hour to 60 or more hours (FIG. 5A) or from 0 hour to 20 or more hours (FIG. 5B).

FIG. 6A and FIG. 6B illustrates that SIRPA KO macrophages derived from SIRPA KO iPSCs had increased killing relative to wild-type control macrophages derived from TC-1133 iPSCs that expressed SIRPA, with the addition of 0.1 μg/mL and 0.3 μg/mL rituximab in Raji cells (FIG. 6A), and with the addition of 10 μg/mL trastuzumab in BT474 cells (FIG. 6B).

FIG. 7A and FIG. 7B illustrate that SIRPA KO macrophages derived from iPSCs had increased killing on Raji cells relative to wild-type control macrophages derived from TC-1133 iPSCs that expressed SIRPA, with the addition of 0.1 μg/mL and 0.3 μg/mL rituximab.

FIG. 8A and FIG. 8B illustrate a schema of iMACs pre-complexed with rituximab (RTX) (FIG. 8A), and in vivo results that demonstrated that the iMACs pre-complexed with rituximab (RTX) killed Raji cells (FIG. 8B).

FIG. 9 illustrates the killing of CAL27 cells, which are head and neck cancer cells, by SIRPA KO macrophages derived from iPSCs (indicated as “SIRPα KO” in the figure, with the addition of 10 μg/mL cetuximab (anti-EGFR antibody), 10 μg/mL secukinumab (anti-TROP2 antibody), or both.

FIG. 10 illustrates the serial killing of LNCap cells (target cells) by SIRPA KO macrophages derived from iPSCs that expressed an anti-PSMA CAR (indicated as “SIRPα KO CAR iMACs” in the figure), SIRPA KO macrophages derived from iPSCs that expressed an anti-CLDN18.2 CAR (indicated as “SIRPα KO iMACs w/irrelevant CAR” in the figure), wild-type control monocytes derived from TC-1133 iPSCs that expressed SIRPA and the anti-PSMA CAR (indicated as “WT CAR iMACs” in the figure), wild-type control monocytes derived from TC-1133 iPSCs that expressed SIRPA but no CAR (indicated as “WT iMACs” in the figure). LNCap cells only group without any macrophages (indicated as “TARGET ONLY” in the figure) was used as the negative control.

FIG. 11 illustrates the tumor growth curve in mice treated with saline vehicle (“Vehicle Control”), wild-type control macrophage precomplexed with rituximab (“WT iMAC/RTX”), wild-type control macrophage precomplexed with rituximab and an anti-CD47 antibody (“WT iMAC/RTX+aCD47”), and SIRPA KO macrophages precomplexed with rituximab (“SIRPA KO iMAC/RTX”). The tumor size was measured by the in vivo bioluminescence imaging. The two dotted vertical lines indicated the days where the vehicles or macrophages were administered to the mice.

### 5. DETAILED DESCRIPTION

The present disclosure is directed, in part, to methods and compositions for generating modified pluripotent cells, such as modified pluripotent cells comprising a polynucleotide or a polypeptide encoding a chimeric antigen receptor (CAR) and genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. Also provided herein are methods and compositions for homogenous populations of modified myeloid progenitor cells, modified monocytes, modified macrophages, and modified CD11b⁺CD45⁺ cells (e.g., CD11b⁺CD45⁺CD14⁻ cells or CD11b⁺CD45⁺CD14⁺ cells) derived from said modified pluripotent cells comprising a polynucleotide or a polypeptide encoding a chimeric antigen receptor (CAR) and genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene.

Unless defined otherwise, all technical and scientific terms used herein have the same meaning as commonly understood to one of ordinary skill in the art to which the invention pertains. Otherwise, certain terms used herein have the meanings as set forth in the specification. All patents, published patent applications and publications cited herein are incorporated by reference as if set forth fully herein. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

As used herein, the abbreviations for the genetically encoded amino acids are conventional and are as follows:

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

It should be noted that as used herein and in the appended claims, the singular forms “a,” “an,” and “the” include plural reference unless the context clearly dictates otherwise.

As used herein, the terms “about” and “approximately” mean within 20%, within 15%, within 10%, within 9%, within 8%, within 7%, within 6%, within 5%, within 4%, within 3%, within 2%, within 1%, or less of a given value or range.

As used herein the term “non-lymphoid intracellular domain” is intended to mean a signaling or costimulatory domain that is not part of the native T cell receptor complex (e.g., CD3ζ, CD3δ, or CD3ε) native B cell receptor complex (e.g. CD22, CD79a, CD79b), or involved in the classical T cell signaling pathway, such as a co-stimulatory molecule from the CD28 family (e.g., CD28 or ICOS) or tumor necrosis factor receptor (TNFR) family (e.g., 4-1BB, OX40, or CD27).

As used herein, the term “myeloid specific promoter” is intended to mean a promoter that enhances or facilitates transcription of a gene in a myeloid cell (e.g., a macrophage or monocyte), relative to a non-myeloid cell as measured by, for example, a reporter gene assay (e.g., a luciferase, or chemiluminescent reporter assay) or measure of the downstream protein encoded by the gene. A representative non-myeloid reference cell may be a HeLa or a 293T cell.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and II (1985); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and/or Janeway, et al. Immunobiology (Garland Science, 7th ed. 2008).

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components in any combination, whether or not a particular combination is expressly described.

### 5.1. Modified Cells

As provided herein, in some aspects the present disclosure involves modified cells comprising genetic disruption of (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cells further comprise (a) a chimeric antigen receptor (“CAR”) (such as a CAR described in Section 5.3), (b) a polynucleotide encoding a CAR (such as a polynucleotide described in Section 5.4) (c) a vector comprising a polynucleotide encoding a CAR (such as a vector described in Section 5.5), or (d) a CAR polypeptide (such as a polypeptide described in Section 5.6). In specific embodiments, the modified cells provided herein are generated according to the methods provided herein, such as the methods of generating a modified cell described in Section 5.2. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified cell comprises genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified cell comprises genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified cell using the SLEEK (SeLection by Essential-gene Exon Knock in) technology, which is described in Allen et al., A highly efficient transgene knock-in technology in clinically relevant cell types. Nat Biotechnol (2023) and International Patent Publication WO2022235811, the content of each of which is incorporated by reference in its entirety. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In some embodiments, the modified cells provided herein comprise genetic disruption of SIRPA (e.g., Homo sapiens NCBI Gene ID: 140885, Mus musculus NCBI Gene ID: 19261). SIRPA encodes for a SIRPα polypeptide, an immunoglobulin-like cell surface receptor for CD47. In certain embodiments, genetic disruption of SIRPA prevents or reduces expression of a SIRPα polypeptide capable of interaction with CD47. In certain embodiments, genetic disruption of SIRPA prevents a SIRPα polypeptide from being capable of interaction with CD47. In some embodiments, the SIRPA gene is a human SIRPA gene. In some embodiments, the SIRPA gene is a non-human SIRPA gene (e.g., a mouse SIRPA gene).

In some embodiments, the modified cells provided herein comprise genetic disruption of CISH (e.g., Homo sapiens NCBI Gene ID: 1154, Mus musculus NCBI Gene ID: 12700). CISH is an inhibitory immune checkpoint gene. In certain embodiments, genetic disruption of CISH prevents or reduces expression of a CISH polypeptide. In some embodiments, the CISH gene is a human CISH gene. In some embodiments, the CISH gene is a non-human CISH gene (e.g., a mouse CISH gene).

In some embodiments, genetic disruption of CISH is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of CISH is performed using gRNA. In certain embodiments, genetic disruption of CISH is performed using gRNA and a Cas12a protein. In certain embodiments, genetic disruption is performed using gRNA and a MAD7 protein. In some embodiments, the gRNA comprises a targeting sequence complementary to a CISH target sequence. In some embodiments, the CISH target sequence comprises coding sequence, for example CISH mRNA sequence. In some embodiments, the gRNA targets exon 1 of CISH. In some embodiments, the gRNA targets exon 2 of CISH. In some embodiments, the gRNA targets exon 3 of CISH. In some embodiments, the gRNA targets exon 4 of CISH. In some embodiments, the CISH target sequence comprises non-coding sequence. Exemplary non-coding sequence includes CISH intronic, promoter, 5′ untranslated region (UTR), 3′ UTR, or enhancer sequence. In some embodiments the gRNA is compatible with a gRNA/Cas9 system. In some embodiments the gRNA is compatible with a gRNA/Cas12a (e.g., MAD7) system. In some embodiments, genetic disruption of CISH is performed by RNAi. In some embodiments, genetic disruption of CISH is performed by conditional knockout. Non-limiting examples of CISH inhibitors, including CISH guide RNAs (gRNAs), include those provided in WO 2017/100861, WO 2017/023803, WO 2018/075664, WO 2019/213610, and WO 2019/217956, each of which are incorporated by reference in its entirety. In some embodiments, the genetic disruption of CISH is performed in human cells.

In some embodiments, the modified cells provided herein comprise genetic disruption of SIGLEC10 (e.g., Homo sapiens NCBI Gene ID: 89790, Mus musculus NCBI Gene ID: 243958). SIGLEC10 is a ligand for CD52, vascular adhesion protein 1 (VAP-1), and CD24. The CD24-SIGLEC10 interaction can serve as an anti-phagocytic signal (see, e.g., Barkal A A, et al., Nature. 2019 August; 572(7769):392-396). Thus, in some embodiments genetic disruption of SIGLEC10 prevents or reduces expression of a SIGLEC10 polypeptide capable of interaction with CD24 on a target cell. In some embodiments genetic disruption of SIGLEC10 prevents a SIGLEC10 polypeptide from being capable of interaction with CD24 on a target cell. In some embodiments, the SIGLEC10 gene is a human SIGLEC10 gene. In some embodiments, the SIGLEC10 gene is a non-human SIGLEC10 gene (e.g., a mouse SIGLEC10 gene).

In some embodiments, genetic disruption of SIGLEC10 is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of SIGLEC10 is performed using gRNA. In certain embodiments, genetic disruption of SIGLEC10 is performed using gRNA and a Cas12a protein. In certain embodiments, genetic disruption is performed using gRNA and a MAD7 protein. In some embodiments, the gRNA comprises a targeting sequence complementary to a SIGLEC10 target sequence. In some embodiments, the SIGLEC10 target sequence comprises coding sequence, for example SIGLEC10 mRNA sequence. In some embodiments, the gRNA targets exon 1 of SIGLEC10. In some embodiments, the gRNA targets exon 2 of SIGLEC10. In some embodiments, the gRNA targets exon 3 of SIGLEC10. In some embodiments, the gRNA targets exon 4 of SIGLEC10. In some embodiments, the gRNA targets exon 5 of SIGLEC10. In some embodiments, the gRNA targets exon 6 of SIGLEC10. In some embodiments, the gRNA targets exon 7 of SIGLEC10. In some embodiments, the gRNA targets exon 8 of SIGLEC10. In some embodiments, the gRNA targets exon 9 of SIGLEC10. In some embodiments, the gRNA targets exon 10 of SIGLEC10. In some embodiments, the gRNA targets exon 11 of SIGLEC10. In some embodiments, the SIGLEC10 target sequence comprises non-coding sequence. Exemplary non-coding sequence includes SIGLEC10 intronic, promoter, 5′ untranslated region (UTR), 3′ UTR, or enhancer sequence. In some embodiments, genetic disruption of SIGLEC10 is performed by RNAi. In some embodiments, genetic disruption of SIGLEC10 is performed by conditional knockout. In some embodiments, the genetic disruption of SIGLEC10 is performed in human cells.

In certain embodiments, the modified cell provided herein is a hypoimmunogenic cell that can evade or partially evade an allogeneic host versus graft immune response. In certain embodiments, the modified cell further comprises (i) genetic disruption of a beta-2-microglobulin (B2M) gene, (ii) genetic disruption of a class II major histocompatibility complex transactivator (CIITA) gene, (iii) genetic disruption of a regulatory factor X (RFX) gene, and/or (iv) an exogenous polynucleotide encoding major histocompatibility complex, class I, E (HLA-E). In certain embodiments, the modified cell provided herein further comprises genetic disruption of a B2M gene. In certain embodiments, the modified cell provided herein further comprises genetic disruption of a CIITA gene. In certain embodiments, the modified cell provided herein further comprises genetic disruption of an RFX gene. In certain embodiments, the modified cell provided herein further comprises genetic disruption of a B2M gene and a CIITA gene. In certain embodiments, the modified cell provided herein further comprises genetic disruption of a B2M gene and an RFX gene. In certain embodiments, the modified cell provided herein further comprises genetic disruption of a CIITA gene and an RFX gene. In certain embodiments, the modified cell provided herein further comprises genetic disruption of a B2M gene, a CIITA gene and an RFX gene. In certain embodiments, the modified cell provided herein further comprises an exogenous polynucleotide encoding HLA-E. In certain embodiments, the modified cell provided herein further comprises genetic disruption of (i) a B2M gene, a CIITA gene and/or an RFX gene, and (ii) an exogenous polynucleotide encoding HLA-E. In certain embodiments, the modified cell provided herein further comprises genetic disruption of a B2M gene and a CIITA gene (e.g., B2M/CIITA double knockout), and an exogenous polynucleotide encoding HLA-E. In certain embodiments, the modified cell provided herein further comprises genetic disruption of a B2M gene, a CIITA gene, and an RFX gene, and an exogenous polynucleotide encoding HLA-E.

In certain embodiments, the modified cell provided herein further comprises genetic disruption of a B2M gene. The B2M gene (e.g., Homo sapiens NCBI Gene ID: 567, Mus musculus NCBI Gene ID: 12010) encodes the beta chain component of MHC class I molecules. Expression of B2M protein is necessary for assembly and function of MHC class I molecules on the cell surface. In certain embodiments, genetic disruption of the B2M gene eliminates or reduces the expression of an B2M protein. In certain embodiments, the B2M gene is a human B2M gene. In certain embodiments, the B2M gene is a non-human B2M gene (e.g., a mouse B2M gene).

In certain embodiments, the modified cell provided herein further comprises genetic disruption of a CIITA gene. The CIITA gene (e.g., Homo sapiens NCBI Gene ID: 4261, Mus musculus NCBI Gene ID: 12265) encodes a CIITA protein that is essential for transcriptional activity of the HLA class II promoter. In certain embodiments, genetic disruption of the CIITA gene eliminates or reduces the expression of a CIITA protein. In certain embodiments, the CIITA gene is a human CIITA gene. In certain embodiments, the CIITA gene is a non-human CIITA gene (e.g., a mouse CIITA gene).

In certain embodiments, the modified cell provided herein further comprises genetic disruption of an RFX gene. In certain embodiments, the RFX gene is an RFX1 gene, an RFX2 gene, an RFX3 gene, an RFX4 gene, an RFX5 gene, an RFX6 gene, an RFX7 gene, or an RFX8 gene. In certain embodiments, genetic disruption of the RFX gene eliminates or reduces the expression of an RFX protein. In certain embodiments, the RFX gene is a human RFX gene. In certain embodiments, the RFX gene is a non-human RFX gene (e.g., a mouse RFX gene).

In certain embodiments, the modified cell provided herein further comprises an exogenous polynucleotide encoding a major histocompatibility complex, class I, E (HLA-E). HLA-E belongs to the HLA class I heavy chain paralogues. HLA-E binds a restricted subset of peptides derived from the leader peptides of other class I molecules. In certain embodiments, a polynucleotide encoding an HLA-E is integrated into (a) a SIRPA gene; (b) a CISH gene; and/or (c) a SIGLEC10 gene, and the modified cell comprises genetic disruption of the gene(s) in which the polynucleotide encoding the HLA-E is integrated. In certain embodiments, a polynucleotide encoding the HLA-E is integrated into (a) a B2M gene; (b) a CIITA gene; and/or (c) an RFX gene, and the modified cell comprises genetic disruption of the gene(s) in which the polynucleotide encoding the HLA-E is integrated. In certain embodiments, a polynucleotide encoding the HLA-E is integrated into a safe harbor locus, e.g., an AAVS1 locus.

In certain embodiments, the modified cell provided herein is homozygous for the genetic disruption of the B2M gene, CIITA gene, and/or RFX gene. In certain embodiments, the modified cell provided herein is heterozygous for the genetic disruption of the B2M gene, CIITA gene, and/or RFX gene.

In certain embodiments, genetic disruption of a B2M gene, a CIITA gene, and/or an RFX gene is performed by a genome editing tool, such as TALEN, ZFN, or CRISPR (e.g., by deletion of one or more exons, introduction of a stop codon, introduction of a null mutation or inactivation of the promoter). In certain embodiments, genetic disruption of the B2M gene, CIITA gene, and/or RFX gene is performed using gRNA. In certain embodiments, genetic disruption of the B2M gene, CIITA gene, and/or RFX gene is performed using gRNA and a Cas12a protein. In certain embodiments, genetic disruption of the B2M gene, CIITA gene, and/or RFX gene is performed using gRNA and a MAD7 protein. In certain embodiments, the gRNA comprises a targeting sequence complementary to a target sequence of the B2M gene, CIITA gene, and/or RFX gene. In certain embodiments, the target sequence comprises a coding sequence of the B2M gene, CIITA gene, and/or RFX gene, for example mRNA sequence. In certain embodiments, the gRNA targets an exon of the B2M gene, CIITA gene, and/or RFX gene. In certain embodiments, the target sequence comprises non-coding sequence. Exemplary non-coding sequence includes intronic, promoter, 5′ untranslated region (UTR), 3′ UTR, or enhancer sequence of the B2M gene, CIITA gene, and/or RFX gene. In certain embodiments, genetic disruption of the B2M gene, CIITA gene, and/or RFX gene is performed by RNAi. In certain embodiments, genetic disruption of the B2M gene, CIITA gene, and/or RFX gene is performed by conditional knockout. In certain embodiments, the genetic disruption of the B2M gene, CIITA gene, and/or RFX gene is performed in human cells.

In certain aspects, the present disclosure relates to modified mammalian cells. In some embodiments, provided herein are modified human cells. In some embodiments, the modified human cells are autologous or allogeneic to a subject receiving administration of the modified cells. In some embodiments, the modified cells are allogeneic, relative to the subject. In other embodiments, the modified cells are autologous, relative to the subject. In some embodiments, provided herein are modified non-human mammalian cells. In certain embodiments, the modified cell is a rodent, e.g., mouse, cell.

### 5.1.1. Modified Pluripotent Cells

As provided herein, the present disclosure relates, in part, to modified pluripotent cells. Pluripotent cells can give rise to a multiplicity of mammalian cell types, and include, for example, induced pluripotent stem cells (iPSCs). In certain embodiments, pluripotent cells do not include embryonic stem cells. In some embodiments, the modified pluripotent cell provided herein is an iPSC. In some embodiments, the modified pluripotent cell provided herein is a hematopoietic stem cell. In some embodiments, a hematopoietic stem cell is produced by a modified iPSC as described herein.

In certain embodiments, the iPSC has been reprogrammed from a cell selected from the group consisting of a peripheral blood mononuclear cell (PBMC), CD34⁺ cord blood, a macrophage, a monocyte, and a fibroblast. In some embodiments, the iPSC has been reprogrammed from a PBMC. In some embodiments, the iPSC has been reprogrammed from CD34⁺ cord blood. In some embodiments, the iPSC is a TC-1133 iPSC. In some embodiments, the iPSC has been reprogrammed from a macrophage. In some embodiments, the iPSC has been reprogrammed from a monocyte. In some embodiments, the iPSC has been reprogrammed from a fibroblast.

In certain embodiments, a pluripotent stem cell as described herein is a mammalian pluripotent cell, e.g., a mammalian iPSC. In certain embodiments, a pluripotent stem cell as described herein is a human pluripotent cell, e.g., a human iPSC. In certain embodiments, a pluripotent cell as described herein is a multipotent cell, e.g., a human multipotent cell. In certain embodiments, a pluripotent cell as described herein is a human hematopoietic stem cell. In certain embodiments, a pluripotent cell as described herein is an embryonic stem cell (ESC), for example, a human ESC. In certain non-limiting embodiments, a pluripotent cell as described herein is a parthenogenic stem cell, e.g., a human parthenogenetic stem cell, a primordial germ cell-like pluripotent stem cell, e.g., a human primordial germ cell-like pluripotent stem cell, an epiblast stem cell, e.g., a human epiblast stem cell, an F-class pluripotent stem cell, e.g., a human F-class pluripotent stem cell, a somatic stem cell, e.g., a human somatic stem cell, or any other cell, e.g., human cell, capable of lineage specific differentiation. In certain embodiments, a pluripotent cell described herein is a non-human pluripotent cell. In certain embodiments, a pluripotent cell as described herein is a nonhuman primate pluripotent cell. In certain embodiments, a pluripotent cell as described herein is a rodent, e.g., mouse, pluripotent cell.

In certain embodiments, a pluripotent cell as described herein is not capable of differentiating or developing into all cell types. For example, in certain embodiments, a human pluripotent cell as described herein is not capable of differentiating or developing into all cell types of the human body.

As provided herein, the modified pluripotent cells of the present disclosure can give rise, for example, to modified myeloid progenitor cells, modified monocytes, modified macrophages, or modified CD11b⁺CD45⁺ cells (e.g., CD11b⁺CD45⁺CD14⁻ cells or CD11b⁺CD45⁺CD14⁺ cells). In some embodiments, the modified pluripotent cells of the present disclosure give rise to modified myeloid progenitor cells. In some embodiments, the modified pluripotent cells of the present disclosure give rise to modified monocytes. In some embodiments, the modified pluripotent cells of the present disclosure give rise to modified macrophages. In some embodiments, the modified pluripotent cells of the present disclosure give rise to modified CD11b⁺CD45⁺ cells. In some embodiments, the modified pluripotent cells of the present disclosure give rise to modified CD11b⁺CD45⁺CD14⁺ cells. In some embodiments, the modified pluripotent cells of the present disclosure give rise to modified CD11b⁺CD45⁺CD14⁻ cells.

In some embodiments, the modified pluripotent cells of the present disclosure give rise to a committed progenitor cell. In some embodiments, the modified pluripotent cells of the present disclosure give rise to modified granulocyte-monocyte progenitor (GMP) cells. In some embodiments, the modified pluripotent cells of the present disclosure give rise to modified monocyte-dendritic cell progenitors (MDPs). In some embodiments, the modified pluripotent cells of the present disclosure give rise to modified monocyte progenitor cells. In some embodiments, any of the modified cells provided herein can be enriched into a homogenous population. For example, a homogeneous population may be produced using and/or may be assessed via markers known in the art (such as those described in Section 5.10.2).

In some aspects, provided herein is a modified pluripotent cell comprising genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified pluripotent cell comprises genetic disruption of the SIGLEC10 gene. In some embodiments, the modified pluripotent cell further comprises genetic disruption of the SIRPA gene and/or the CISH gene. For example, in certain embodiments, the modified pluripotent cell comprises genetic disruption of SIGLEC10 and SIRPA. In other embodiments, the modified pluripotent cell comprises genetic disruption of SIGLEC10 and CISH. In some embodiments, the modified pluripotent cell comprises genetic disruption of SIRPA and CISH. In still further embodiments, the modified pluripotent cell comprises genetic disruption of SIGLEC10, SIRPA, and CISH. In some embodiments, the modified pluripotent cell is heterozygous for the genetic disruption of the SIGLEC10 gene. In some embodiments, the modified pluripotent cell is homozygous for the genetic disruption of the SIGLEC10 gene. In some embodiments, the modified pluripotent cell is heterozygous for the genetic disruption of the SIRPA gene. In some embodiments, the modified pluripotent cell is homozygous for the genetic disruption of the SIRPA gene. In some embodiments, the modified pluripotent cell is heterozygous for the genetic disruption of the CISH gene. In some embodiments, the modified pluripotent cell is homozygous for the genetic disruption of the CISH gene.

In some aspects, provided herein is a modified pluripotent cell comprising a polynucleotide or a polypeptide encoding a chimeric antigen receptor (CAR) (such as a CAR described in Section 5.3) and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified pluripotent cell comprises genetic disruption of the SIGLEC10 gene. In some embodiments, the modified pluripotent cell is heterozygous for the genetic disruption of the SIGLEC10 gene. In some embodiments, the modified pluripotent cell is homozygous for the genetic disruption of the SIGLEC10 gene. In some embodiments, the modified pluripotent cell comprises genetic disruption of the SIRPA gene. In some embodiments, the modified pluripotent cell is heterozygous for the genetic disruption of the SIRPA gene. In some embodiments, the modified pluripotent cell is homozygous for the genetic disruption of the SIRPA gene. In some embodiments, the modified pluripotent cell comprises genetic disruption of the CISH gene. In some embodiments, the modified pluripotent cell is heterozygous for the genetic disruption of the CISH gene. In some embodiments, the modified pluripotent cell is homozygous for the genetic disruption of the CISH gene. In certain embodiments, the modified pluripotent cell comprises genetic disruption of SIGLEC10 and SIRPA. In other embodiments, the modified pluripotent cell comprises genetic disruption of SIGLEC10 and CISH. In some embodiments, the modified pluripotent cell comprises genetic disruption of SIRPA and CISH. In still further embodiments, the modified pluripotent cell comprises genetic disruption of SIGLEC10, SIRPA, and CISH. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified pluripotent cell comprises genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified pluripotent cell comprises genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified pluripotent cell using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In certain embodiments, the modified pluripotent cell further comprises (i) genetic disruption of a B2M gene, (ii) genetic disruption of a CIITA gene, (iii) genetic disruption of an RFX gene, and/or (iv) an exogenous polynucleotide encoding HLA-E. In certain embodiments, the modified pluripotent cell provided herein further comprises genetic disruption of (i) a B2M gene, a CIITA gene and/or an RFX gene, and (ii) an exogenous polynucleotide encoding HLA-E. In certain embodiments, the modified pluripotent cell provided herein further comprises genetic disruption of a B2M gene and a CIITA gene (e.g., B2M/CIITA double knockout), and an exogenous polynucleotide encoding HLA-E. In certain embodiments, the modified pluripotent cell provided herein further comprises genetic disruption of a B2M gene, a CIITA gene, and an RFX gene, and an exogenous polynucleotide encoding HLA-E.

In certain embodiments, the modified pluripotent cell provided herein is homozygous for the genetic disruption of the B2M gene, CIITA gene, and/or RFX gene. In certain embodiments, the modified pluripotent cell provided herein is heterozygous for the genetic disruption of the B2M gene, CIITA gene, and/or RFX gene.

### 5.1.2. Modified Myeloid Progenitor Cells

The present disclosure relates, in part, to modified myeloid progenitor cells derived from a modified pluripotent cell (such as a modified pluripotent cell described in Section 5.1.1). For example, in some embodiments, the modified pluripotent cell is generated according to the methods provided herein, such as the methods described in Section 5.2.1, and the modified pluripotent cell is differentiated into a myeloid progenitor cell. Techniques known to one of skill in the art or described herein (such as in Section 5.8) may be used to differentiate a pluripotent cell into a myeloid progenitor cell. For example, a homogeneous population of modified myeloid progenitor cells may be produced using and/or may be assessed via markers known in the art (such as those described in Section 5.10.2).

A myeloid progenitor cell described herein can give rise to a monocyte and/or macrophage, or progenitors thereof. A modified myeloid progenitor cell described herein can give rise to a modified monocyte and/or modified macrophage as described herein, or progenitors thereof. In certain embodiments, the modified myeloid progenitor cell is a human myeloid progenitor cell. In certain embodiments, the modified myeloid progenitor cell is a mammalian myeloid progenitor cell. In certain embodiments, the modified myeloid progenitor cell is a primate myeloid progenitor cell. In certain embodiments, the modified myeloid progenitor cell is a non-human primate myeloid progenitor cell. In certain embodiments, the modified myeloid progenitor cell is a rodent, e.g., mouse, myeloid progenitor cell.

In certain embodiments, a modified myeloid progenitor cell, as presented herein, expresses a detectable level of a myeloid progenitor cell marker, such as for example CD34, and CD38, and does not express a detectable level of Lin and/or CD45RA (e.g., Lin⁻ CD34⁺CD38⁺CD45RA⁻ cells), or any other myeloid progenitor cell marker(s) known to one of skill in the art or described herein (such as in Section 5.10.2).

In some aspects, provided herein is a homogeneous population of modified myeloid progenitor cells comprising a polynucleotide or a polypeptide encoding a chimeric antigen receptor (CAR) (such as a CAR described in Section 5.3) and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified myeloid progenitor cells comprise genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified myeloid progenitor cells comprise genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified myeloid progenitor cell using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In some embodiments, the homogeneous population of modified myeloid progenitor cells comprises genetic disruption of the SIRPA gene. In some embodiments, the modified myeloid progenitor cells are heterozygous for the genetic disruption of the SIRPA gene. In some embodiments, the modified myeloid progenitor cells are homozygous for the genetic disruption of the SIRPA gene. In certain embodiments, the homogeneous population of modified myeloid progenitor cells are isogenic.

In some embodiments, the homogeneous population of modified myeloid progenitor cells comprises genetic disruption of the SIGLEC10 gene. In some embodiments, the modified myeloid progenitor cells are heterozygous for the genetic disruption of the SIGLEC10 gene. In some embodiments, the modified myeloid progenitor cells are homozygous for the genetic disruption of the SIGLEC10 gene. In certain embodiments, the homogeneous population of modified myeloid progenitor cells are isogenic.

In some embodiments, the homogeneous population of modified myeloid progenitor cells comprises genetic disruption of the CISH gene. In some embodiments, the modified myeloid progenitor cells are heterozygous for the genetic disruption of the CISH gene. In some embodiments, the modified myeloid progenitor cells are homozygous for the genetic disruption of the CISH gene. In certain embodiments, the homogeneous population of modified myeloid progenitor cells are isogenic.

In specific embodiments, the homogeneous population of modified myeloid progenitor cells comprises genetic disruption of SIRPA and CISH. In certain embodiments, the homogeneous population of modified myeloid progenitor cells comprises genetic disruption of SIRPA and SIGLEC10. In further embodiments, the homogeneous population of modified myeloid progenitor cells comprises genetic disruption of SIGLEC10 and CISH. In still further embodiments, the homogeneous population of modified myeloid progenitor cells comprises genetic disruption of SIGLEC10, SIRPA, and CISH. In certain embodiments, the homogeneous population of modified myeloid progenitor cells are isogenic.

In certain embodiments, the modified myeloid progenitor cell further comprises (i) genetic disruption of a B2M gene, (ii) genetic disruption of a CIITA gene, (iii) genetic disruption of an RFX gene, and/or (iv) an exogenous polynucleotide encoding HLA-E. In certain embodiments, the modified myeloid progenitor cell provided herein further comprises genetic disruption of (i) a B2M gene, a CIITA gene and/or an RFX gene, and (ii) an exogenous polynucleotide encoding HLA-E. In certain embodiments, the modified myeloid progenitor cell provided herein further comprises genetic disruption of a B2M gene and a CIITA gene (e.g., B2M/CIITA double knockout), and an exogenous polynucleotide encoding HLA-E. In certain embodiments, the modified myeloid progenitor cell provided herein further comprises genetic disruption of a B2M gene, a CIITA gene, and an RFX gene, and an exogenous polynucleotide encoding HLA-E.

In certain embodiments, the modified myeloid progenitor cell provided herein is homozygous for the genetic disruption of the B2M gene, CIITA gene, and/or RFX gene. In certain embodiments, the modified myeloid progenitor cell provided herein is heterozygous for the genetic disruption of the B2M gene, CIITA gene, and/or RFX gene.

In some embodiments, the population of modified myeloid progenitor cells are derived from induced pluripotent stem cells (iPSCs). In certain embodiments, the iPSCs have been reprogrammed from a cell selected from the group consisting of a peripheral blood mononuclear cell (PBMC), CD34⁺ cord blood, a macrophage, a monocyte, and a fibroblast. In some embodiments, the iPSCs have been reprogrammed from a PBMC. In some embodiments, the iPSCs have been reprogrammed from CD34⁺ cord blood. In some embodiments, the iPSCs have been reprogrammed from a macrophage. In some embodiments, the iPSCs have been reprogrammed from a monocyte. In some embodiments, the iPSCs have been reprogrammed from a fibroblast. Techniques known to one of skill in the art or described herein (such as in Section 5.8) may be used to differentiate a pluripotent cell into a myeloid progenitor cell. In some embodiments, the iPSC is a TC-1133 iPSC.

The present disclosure provides a population of cells comprising a modified myeloid progenitor cell as described herein. In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2). In some embodiments, 100% of the cells express a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2).

### 5.1.3. Modified Monocytes

The present disclosure relates, in part, to modified monocytes derived from a modified pluripotent cell (such as a modified pluripotent cell described in Section 5.1.1). For example, in some embodiments, the modified pluripotent cell is generated according to the methods provided herein, such as the methods described in Section 5.2.1, and the modified pluripotent cell is differentiated into a monocyte. In some embodiments, the modified monocyte provided herein is derived from a modified myeloid progenitor cell (such as a modified myeloid progenitor cell described in Section 5.1.2). For example, in some embodiments, the modified myeloid progenitor cell is generated according to the methods provided herein (such as the methods described in Section 5.2.2), and the modified myeloid progenitor cell is differentiated into a monocyte. Techniques known to one of skill in the art or described herein (such as in Section 5.8) may be used to differentiate a pluripotent cell into a monocyte, and/or a myeloid progenitor cell into a monocyte. For example, a homogeneous population of modified monocytes cells may be produced using and/or may be assessed via markers known in the art (such as those described in Section 5.10.2).

In certain embodiments, the modified monocyte is a human monocyte. In certain embodiments, the modified monocyte is a mammalian monocyte. In certain embodiments, the modified monocyte is a primate monocyte. In certain embodiments, the modified monocyte is a non-human primate monocyte. In certain embodiments, the modified monocyte is a rodent, e.g., mouse, monocyte.

In certain embodiments, a modified monocyte exhibits a monocyte phenotype such as a monocyte phenotype as described in Section 5.10.2. For example, in certain embodiments, a modified monocyte disclosed herein is a non-adherent cell. In certain embodiments, a modified monocyte disclosed herein has a monocyte cell morphology, e.g., the size of the cell is smaller than macrophages, but larger than T-cells, B-cells or natural killer (NK) cells. In certain embodiments, a modified monocyte has a cell morphology that is less granular than a macrophage, e.g., a mature macrophage. In certain embodiments, a modified monocyte disclosed herein is a non-adherent cell that has a cell morphology that is less granular than a macrophage, e.g., a mature macrophage and has a cell size that is smaller than macrophages, but larger than T-cells, B-cells or NK cells.

In certain embodiments, a modified monocyte presented herein expresses a detectable level of CD11b and CD45 (e.g., is a CD11b⁺CD45⁺ cell). In certain embodiments, a modified monocyte disclosed herein expresses a detectable level of CD14, e.g., is CD11b⁺CD45⁺CD14⁺. In certain embodiments, a modified monocyte presented herein does not express a detectable level of CD14, e.g., is CD11b⁺CD45⁺CD14⁻. In certain embodiments, a modified monocyte presented herein expresses a high level of CD14 and does not express a detectable level of CD16 (CD14^{high} CD16⁻). In certain embodiments, a modified monocyte presented herein expresses a high level of CD14 and a detectable level of CD16 (CD14^{high} CD16⁺). In certain embodiments, a modified monocyte presented herein express a low level of CD14 and a high detectable level of CD16 (CD14^{low} CD16^{high}) expression. The present disclosure provides a population of cells comprising a modified monocyte as described herein. In certain embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses one or more such levels.

In certain embodiments, a modified monocyte disclosed herein has the properties of killing target cells (e.g., via CAR or monoclonal antibody (mAb) targeting), phagocytosing particles (e.g., bacteria), and/or migrating towards chemokines. In certain embodiments, a modified monocyte disclosed herein exhibits increased killing of target cells (e.g., via CAR or monoclonal antibody (mAb) targeting), phagocytosing particles (e.g., bacteria), and/or migrating towards chemokines relative to a non-modified monocyte. Any assays known in the art can be used for measuring these properties, for example, the assays disclosed in Section 5.10.

In some aspects, provided herein is a homogeneous population of modified monocytes comprising a polynucleotide or a polypeptide encoding a chimeric antigen receptor (CAR) (such as a CAR described in Section 5.3) and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified monocytes comprise genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified monocytes comprise genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified monocyte using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In some embodiments, the homogeneous population of modified monocytes comprises genetic disruption of the SIRPA gene. In some embodiments, the modified monocytes are heterozygous for the genetic disruption of the SIRPA gene. In some embodiments, the modified monocytes are homozygous for the genetic disruption of the SIRPA gene. In certain embodiments, the homogeneous population of modified monocytes are isogenic.

In some embodiments, the homogeneous population of modified monocytes comprises genetic disruption of the SIGLEC10 gene. In some embodiments, the modified monocytes are heterozygous for the genetic disruption of the SIGLEC10 gene. In some embodiments, the modified monocytes are homozygous for the genetic disruption of the SIGLEC10 gene. In certain embodiments, the homogeneous population of modified monocytes are isogenic.

In some embodiments, the homogeneous population of modified monocytes comprises genetic disruption of the CISH gene. In some embodiments, the modified monocytes are heterozygous for the genetic disruption of the CISH gene. In some embodiments, the modified monocytes are homozygous for the genetic disruption of the CISH gene. In certain embodiments, the homogeneous population of modified monocytes are isogenic.

In specific embodiments, the homogeneous population of modified monocytes comprises genetic disruption of SIRPA and CISH. In certain embodiments, the homogeneous population of modified monocytes comprises genetic disruption of SIRPA and SIGLEC10. In further embodiments, the homogeneous population of modified monocytes comprises genetic disruption of SIGLEC10 and CISH. In still further embodiments, the homogeneous population of modified monocytes comprises genetic disruption of SIGLEC10, SIRPA, and CISH. In certain embodiments, the homogeneous population of modified monocytes are isogenic.

In some embodiments, the population of modified monocytes are derived from induced pluripotent stem cells (iPSCs). In certain embodiments, the iPSCs have been reprogrammed from a cell selected from the group consisting of a peripheral blood mononuclear cell (PBMC), CD34⁺ cord blood, a macrophage, a monocyte, and a fibroblast. In some embodiments, the iPSCs have been reprogrammed from a PBMC. In some embodiments, the iPSCs have been reprogrammed from CD34⁺ cord blood. In some embodiments, the iPSCs have been reprogrammed from a macrophage. In some embodiments, the iPSCs are reprogrammed from a monocyte. In some embodiments, the iPSCs have been reprogrammed from a fibroblast. In some embodiments, the iPSC is a TC-1133 iPSC.

In certain embodiments, the modified monocyte further comprises (i) genetic disruption of a B2M gene, (ii) genetic disruption of a CIITA gene, (iii) genetic disruption of an RFX gene, and/or (iv) an exogenous polynucleotide encoding HLA-E. In certain embodiments, the modified monocyte provided herein further comprises genetic disruption of (i) a B2M gene, a CIITA gene and/or an RFX gene, and (ii) an exogenous polynucleotide encoding HLA-E. In certain embodiments, the modified monocyte provided herein further comprises genetic disruption of a B2M gene and a CIITA gene (e.g., B2M/CIITA double knockout), and an exogenous polynucleotide encoding HLA-E. In certain embodiments, the modified monocyte provided herein further comprises genetic disruption of a B2M gene, a CIITA gene, and an RFX gene, and an exogenous polynucleotide encoding HLA-E.

In certain embodiments, the modified monocyte provided herein is homozygous for the genetic disruption of the B2M gene, CIITA gene, and/or RFX gene. In certain embodiments, the modified monocyte provided herein is heterozygous for the genetic disruption of the B2M gene, CIITA gene, and/or RFX gene.

In some embodiments, the population of modified monocytes comprise an agent capable of preventing or reducing interaction between CD47 and SIRPα (see, e.g., WO 2019/241403, incorporated by reference in its entirety). In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody.

In some embodiments, the population of modified monocytes comprises an agent capable of preventing or reducing interaction between CD24 and SIGLEC10 (see, e.g., WO 2019/241403, incorporated by reference in its entirety). In some embodiments, the agent comprises an anti-CD24 antibody. In some embodiments, the agent comprises an anti-SIGLEC10 antibody. In some embodiments, the agent comprises a soluble SIGLEC10 polypeptide.

The present disclosure provides a population of cells comprising a modified monocyte as described herein. In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a monocyte marker, such as for example CD11b⁺CD45⁺, CD14 CD11b⁺CD45⁺, or CD14⁻CD11b⁺CD45⁺, or any other monocyte marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a monocyte marker, such as for example CD11b⁺CD45⁺, CD14⁺CD11b⁺CD45⁺, or CD14⁻CD11b⁺CD45⁺, or any other monocyte marker(s) known to one of skill in the art or described herein (such as in Section 5.10.2). In some embodiments, 100% of the cells express a monocyte marker, such as for example CD11b⁺CD45⁺, CD14⁺CD11b⁺CD45⁺, or CD14⁻CD11b⁺CD45⁺, or any other monocyte marker(s) known to one of skill in the art or described herein (such as in Section 5.10.2).

### 5.1.4. Modified Macrophages

The present disclosure also relates, in part, to a modified macrophage derived from a modified pluripotent cell (such as a modified pluripotent cell described in Section 5.1.1). For example, in some embodiments, the modified pluripotent cell is generated according to the methods provided herein (such as the methods described in Section 5.2.1), and the modified pluripotent cell is differentiated into a macrophage. In some embodiments, the modified macrophage provided herein is derived from a modified myeloid progenitor cell (such as a modified myeloid progenitor cell described in Section 5.1.2). For example, in some embodiments, the modified myeloid progenitor cell is generated according to the methods provided herein (such as the methods described in Section 5.2.2), and the modified myeloid progenitor cell is differentiated into a macrophage. In some embodiments, the modified macrophage provided herein is derived from a modified monocyte (such as a modified monocyte described in Section 5.1.3). For example, in some embodiments, the modified monocyte is generated according to the methods provided herein (such as the methods described in Section 5.2.3), and the modified monocyte is differentiated into a macrophage. Techniques known to one of skill in the art or described herein (such as in Section 5.8) may be used to differentiate a pluripotent cell into a macrophage, a myeloid progenitor cell into a macrophage and/or differentiate a monocyte into a macrophage. For example, a homogeneous population of modified macrophages may be produced using and/or may be assessed via markers known in the art (such as those described in Section 5.10.2).

In certain embodiments, the modified macrophage is a human macrophage. In certain embodiments, the modified macrophage is a mammalian macrophage. In certain embodiments, the modified macrophage is a primate macrophage. In certain embodiments, the modified macrophage is a non-human primate macrophage. In certain embodiments, the modified macrophage is a rodent, e.g., mouse, macrophage.

In certain embodiments, the modified immature macrophage is a human immature macrophage. In certain embodiments, the modified immature macrophage is a mammalian immature macrophage. In certain embodiments, the modified immature macrophage is a primate immature macrophage. In certain embodiments, the modified immature macrophage is a non-human primate immature macrophage. In certain embodiments, the modified immature macrophage is a rodent, e.g., mouse, immature macrophage.

In certain embodiments, a modified macrophage exhibits a macrophage phenotype such as a macrophage phenotype as described in Section 5.10.2. For example, in certain embodiments, a modified macrophage disclosed herein, in a cell culture environment, is an-adherent cell. In certain embodiments, a modified macrophage disclosed herein has a macrophage cell morphology, e.g., the size of the cell is larger than monocytes. In certain embodiments, a modified macrophage has a cell morphology that is more granular than a monocyte. In certain embodiments, a modified macrophage disclosed herein is an adherent cell that has a cell morphology that is more granular than a monocyte and has a cell size that is larger than monocytes.

In certain embodiments, a modified macrophage presented herein expresses a detectable level of CD11b⁺ and CD45⁺ (e.g., is a CD11b⁺CD45⁺ cell). In certain embodiments, a modified macrophage disclosed herein expresses a detectable level of CD14, e.g., is CD11b⁺CD45⁺CD14⁺. In certain embodiments, a modified macrophage presented herein does not express a detectable level of CD14 (e.g., is CD11b⁺CD45⁺CD14⁻).

In certain embodiments, a modified macrophage, e.g., immature macrophage, disclosed herein has the properties of killing target cells (e.g., via CAR or monoclonal antibody (mAb) targeting), phagocytosing particles (e.g., bacteria), and/or migrating towards chemokines. In certain embodiments, a modified macrophage, e.g., immature macrophage, disclosed herein exhibits increased killing of target cells (e.g., via CAR or monoclonal antibody (mAb) targeting), phagocytosing particles (e.g., bacteria), and/or migrating towards chemokines relative to a non-modified macrophage, e.g., immature macrophage. Any assays known in the art can be used for measuring these properties, for example, the assays disclosed in Section 5.10.

In certain embodiments, the modified macrophage provided herein is an immature macrophage. In certain embodiments, the immature macrophage is derived from a pluripotent cell (e.g., an iPSC). In certain embodiments, the immature macrophage has not been subjected to any maturation or polarization process. In certain embodiments, the immature macrophage is less granular and less adherent in cell culture than a mature macrophage. In certain embodiments, the immature macrophage is smaller in cell culture than a mature macrophage. In certain embodiments, the immature macrophage is more granular and bigger than monocytes obtained from blood.

In certain embodiments, the modified macrophage provided herein is a tissue-resident macrophage (e.g., an adipose-associated macrophage, osteoblast, microglia, motile liver macrophage, perivascular macrophage, meningeal macrophage, intestinal macrophage, Kupffer cell, Langerhans cell, alveolar macrophage or red-pulp macrophage).

The present disclosure further provides a population of cells comprising a modified macrophage, e.g., a modified immature macrophage, as described herein. In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a macrophage marker, such as for example CD11b⁺CD45⁺, CD14⁺CD11b⁺CD45⁺, or CD14⁻CD11b⁺CD45⁺, or any other macrophage marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a macrophage marker, such as for example CD11b⁺CD45⁺, CD14⁻CD11b⁺CD45⁺, or CD14⁻CD11b⁺CD45⁺, or any other macrophage marker(s) known to one of skill in the art or described herein (such as in Section 5.10.2). In some embodiments, 100% of the cells express a macrophage marker, such as for example CD11b⁺CD45⁺, CD14⁺CD11b⁺CD45⁺, or CD14⁻CD11b⁺CD45⁺, or any other macrophage marker(s) known to one of skill in the art or described herein (such as in Section 5.10.2).

In some aspects, provided herein is a homogeneous population of modified macrophages comprising a polynucleotide or a polypeptide encoding a chimeric antigen receptor (CAR) (such as a CAR described in Section 5.3) and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified macrophages comprise genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified macrophages comprise genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified macrophage using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In some embodiments, the homogeneous population of modified macrophages comprises genetic disruption of the SIRPA gene. In some embodiments, the modified macrophages are heterozygous for the genetic disruption of the SIRPA gene. In some embodiments, the modified macrophages are homozygous for the genetic disruption of the SIRPA gene. In certain embodiments, the homogeneous population of modified macrophages are isogenic.

In some embodiments, the homogeneous population of modified macrophages comprises genetic disruption of the SIGLEC10 gene. In some embodiments, the modified macrophages are heterozygous for the genetic disruption of the SIGLEC10 gene. In some embodiments, the modified macrophages are homozygous for the genetic disruption of the SIGLEC10 gene. In certain embodiments, the homogeneous population of modified macrophages are isogenic.

In some embodiments, the homogeneous population of modified macrophages comprises genetic disruption of the CISH gene. In some embodiments, the modified macrophages are heterozygous for the genetic disruption of the CISH gene. In some embodiments, the modified macrophages are homozygous for the genetic disruption of the CISH gene. In certain embodiments, the homogeneous population of modified macrophages are isogenic.

In specific embodiments, the homogeneous population of modified macrophages comprises genetic disruption of SIRPA and CISH. In certain embodiments, the homogeneous population of modified macrophages comprises genetic disruption of SIRPA and SIGLEC10. In further embodiments, the homogeneous population of modified macrophages comprises genetic disruption of SIGLEC10 and CISH. In still further embodiments, the homogeneous population of modified macrophages comprises genetic disruption of SIGLEC10, SIRPA, and CISH. In certain embodiments, the homogeneous population of modified macrophages are isogenic.

In certain embodiments, the modified macrophage further comprises (i) genetic disruption of a B2M gene, (ii) genetic disruption of a CIITA gene, (iii) genetic disruption of an RFX gene, and/or (iv) an exogenous polynucleotide encoding HLA-E. In certain embodiments, the modified macrophage provided herein further comprises genetic disruption of (i) a B2M gene, a CIITA gene and/or an RFX gene, and (ii) an exogenous polynucleotide encoding HLA-E. In certain embodiments, the modified macrophage provided herein further comprises genetic disruption of a B2M gene and a CIITA gene (e.g., B2M/CIITA double knockout), and an exogenous polynucleotide encoding HLA-E. In certain embodiments, the modified macrophage provided herein further comprises genetic disruption of a B2M gene, a CIITA gene, and an RFX gene, and an exogenous polynucleotide encoding HLA-E.

In certain embodiments, the modified macrophage provided herein is homozygous for the genetic disruption of the B2M gene, CIITA gene, and/or RFX gene. In certain embodiments, the modified macrophage provided herein is heterozygous for the genetic disruption of the B2M gene, CIITA gene, and/or RFX gene.

In some embodiments, the population of modified macrophages are derived from induced pluripotent stem cells (iPSCs). In certain embodiments, the iPSCs have been reprogrammed from a cell selected from the group consisting of a peripheral blood mononuclear cell (PBMC), CD34⁺ cord blood, a macrophage, a monocyte, and a fibroblast. In some embodiments, the iPSCs have been reprogrammed from a PBMC. In some embodiments, the iPSCs are reprogrammed from CD34⁺ cord blood. In some embodiments, the iPSCs have been reprogrammed from a macrophage. In some embodiments, the iPSCs have been reprogrammed from a monocyte. In some embodiments, the iPSCs have been reprogrammed from a fibroblast. In some embodiments, the iPSC is a TC-1133 iPSC.

In some embodiments, the population of modified macrophages comprise an agent capable of preventing or reducing interaction between CD47 and SIRPα (see, e.g., WO 2019/241403, incorporated by reference in its entirety). In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody.

In some embodiments, the population of modified macrophages comprises an agent capable of preventing or reducing interaction between CD24 and SIGLEC10 (see, e.g., WO 2019/241403, incorporated by reference in its entirety). In some embodiments, the agent comprises an anti-CD24 antibody. In some embodiments, the agent comprises an anti-SIGLEC10 antibody. In some embodiments, the agent comprises a soluble SIGLEC10 polypeptide.

In some embodiments, the macrophage is a pro-inflammatory macrophage (i.e., an M1 macrophage). In some embodiments, the macrophage secretes TNFα, IL-17A, and/or type 1 cytokines (e.g., IL-6, or IL-12). In some embodiments, the macrophage expresses a marker in Table 1. In some embodiments, the macrophage expresses a human M1 macrophage marker selected from CD40, CX3CR1, and CXCR3. In some embodiments, expression of the CAR promotes M1 polarization of the macrophage.

TABLE 1
Human M1 Macrophage Markers

| | | | Marker |
|---|---|---|---|
| Species | Macrophage type | Marker | type |
| Human | M1 | IFN-γ | Secreted |
| Human | M1 | IL-1α | Secreted |
| Human | M1 | IL-1β | Secreted |
| Human | M1 | IL-6 | Secreted |
| Human | M1 | IL-12 | Secreted |
| Human | M1 | IL-23 | Secreted |
| Human | M1 | TNF-α | Secreted |
| Human | M1 | CD16 | Surface |
| Human | M1 | CD16/CD32 | Surface |
| Human | M1 | CD32 | Surface |
| Human | M1 | CD64 | Surface |
| Human | M1 | CD68 | Surface |
| Human | M1 | CD80 | Surface |
| Human | M1 | CD86 | Surface |
| Human | M1 | CD369 (DECTIN- | Surface |
| | | 1) | |
| Human | M1 | Mer (MerTK) | Surface |
| Human | M1 | MHC II | Surface |
| Human | M1 | IRF5 | Intracellular/ |
| | | | transcription |
| | | | factor |
| Human | M1 | STAT1 | Intracellular/ |
| | | | transcription |
| | | | factor |

In some embodiments, the macrophage is not an anti-inflammatory macrophage (i.e., an M2 macrophage). In specific embodiments, the macrophage does not secrete IL-4, IL-13, and/or type 2 cytokines. In some embodiments, the macrophage does not express a marker in Table 2. In some embodiments, the macrophage expresses a human M2 macrophage marker CD36. In specific embodiments, expression of the CAR decreases expression of one of more M2 marker.

TABLE 2
Human Macrophage M2 Markers

| Species | Macrophage type | Marker | Marker type |
|---|---|---|---|
| Human | M2 | IDO | Secreted |
| Human | M2 | IL-10 | Secreted |
| Human | M2 | TGF-b | Secreted |
| Human | M2 | CD115 | Surface |
| Human | M2 | CD204 | Surface |
| Human | M2 | CD163 | Surface |
| Human | M2 | CD206 (MMR) | Surface |
| Human | M2 | CD209 (DC- | Surface |
| | | SIGN) | |
| Human | M2 | FceR1 | Surface |
| Human | M2 | VSIG4 | Surface |
| Human | M2 | IRF4 | Intracellular/transcription |
| | | | factor |
| Human | M2 | STAT6 | Intracellular/transcription |
| | | | factor |

In some embodiments, the macrophage is an anti-inflammatory macrophage (i.e., an M2 macrophage). In specific embodiments, the macrophage secretes IL-4, IL-13, and/or type 2 cytokines. In some embodiments, the macrophage expresses a marker in Table 2. In specific embodiments, expression of the CAR increases expression of one of more M2 marker.

In some embodiments, the macrophage is not a pro-inflammatory macrophage (i.e., an M1 macrophage). In some embodiments, the macrophage does not secrete TNFα, IL-17A, and/or type 1 cytokines (e.g., IL-6, or IL-12). In some embodiments, the macrophage does not express a marker in Table 1. In some embodiments, expression of the CAR promotes M1 polarization of the macrophage.

In some embodiments, the macrophage is not a pro-inflammatory macrophage (i.e., an M1 macrophage) or an anti-inflammatory macrophage (i.e., an M2 macrophage). In some embodiments, the macrophage is an M0 macrophage.

### 5.1.5. Modified CD11b⁺CD45⁺ Cells

The present disclosure also relates, in part, to a modified CD11b⁺CD45⁺ cell. A CD11b⁺CD45⁺ cell can be any cell (e.g., a myeloid lineage cell) that express detectable levels of CD11b and CD45, for example a monocyte, a macrophage, an immature macrophage, a tissue-resident macrophage (e.g., an adipose-associated macrophage, osteoblast, microglia, motile liver macrophage, perivascular macrophage, meningeal macrophage, intestinal macrophage, Kupffer cell, Langerhans cell, alveolar macrophage or red-pulp macrophage), a precursor thereof, or a progenitor thereof.

In certain embodiments, a modified CD11b⁺CD45⁺ cell disclosed herein expresses a detectable level of CD14 (e.g., CD11b⁺CD45⁺CD14⁺). In certain embodiments, the modified CD11b⁺CD45⁺ cell disclosed herein does not express a detectable level of CD14 (e.g., CD11b⁺CD45⁺CD14⁻). In certain embodiments, a modified CD11b⁺CD45⁺CD14⁻ cell disclosed herein can mature into cells that express a detectable level of CD14. In certain embodiments, a modified CD11b⁺CD45⁺CD14⁺ cell presented herein expresses a high level of CD14 and does not express a detectable level of CD16 (CD14^{high} CD16⁻). In certain embodiments, a modified CD11b⁺CD45⁺CD14⁺ cell presented herein expresses a high level of CD14 and a detectable level of CD16 (CD14^{high} CD16⁺). In certain embodiments, a modified CD11b⁺CD45⁺CD14⁺ cell presented herein express a low level of CD14 and a high detectable level of CD16 (CD14^{low} CD16^{high}) expression.

In certain embodiments, a modified CD11b⁺CD45⁺ cell (e.g., a CD11b⁺CD45⁺CD14⁺ cell or a CD11b⁺CD45⁺CD14⁻ cell) disclosed herein has the properties of killing target cells (e.g., via CAR or monoclonal antibody (mAb) targeting), phagocytosing particles (e.g., bacteria), and/or migrating towards chemokines. In certain embodiments, a modified CD11b⁺CD45⁺ cell (e.g., CD11b⁺CD45⁺CD14⁺ cell or CD11b⁺CD45⁺CD14⁻ cell) disclosed herein exhibits increased killing of target cells (e.g., via CAR or monoclonal antibody (mAb) targeting), phagocytosing particles (e.g., bacteria), and/or migrating towards chemokines relative to a non-modified CD11b⁺CD45⁺ cell (e.g., a non-modified CD11b⁺CD45⁺CD14⁺ cell or non-modified CD11b⁺CD45⁺CD14⁻ cell). Any assays known in the art can be used for measuring these properties, for example, the assays disclosed in Section 5.10.

In certain embodiments, the modified CD11b⁺CD45⁺ cell is derived from a modified pluripotent cell (such as a modified pluripotent cell described in Section 5.1.1). In certain embodiments, the modified pluripotent cell is generated according to the methods provided herein (such as the methods described in Section 5.2.1), and the modified pluripotent cell is differentiated into a CD11b⁺CD45⁺ cell. Techniques known to one of skill in the art or described herein (such as in Section 5.8) may be used to differentiate a pluripotent cell into a CD11b⁺CD45⁺ cell. For example, a homogeneous population of modified CD11b⁺CD45⁺ cells or a cell population comprising a modified CD11b⁺CD45⁺ cell disclosed herein (e.g., at least about 95% of the cells in the cell population are the CD11b⁺CD45⁺ cells) may be produced, selected from and/or assessed via CD11b and CD45 markers.

In certain embodiments, the modified CD11b⁺CD45⁺ cell is a human CD11b⁺CD45⁺ cell. In certain embodiments, the modified CD11b⁺CD45⁺ cell is a mammalian CD11b⁺CD45⁺ cell. In certain embodiments, the modified CD11b⁺CD45⁺ cell is a primate CD11b⁺CD45⁺ cell. In certain embodiments, the modified CD11b⁺CD45⁺ cell is a non-human primate CD11b⁺CD45⁺ cell. In certain embodiments, the modified CD11b⁺CD45⁺ cell is a rodent, e.g., mouse, CD11b⁺CD45⁺ cell.

In certain embodiments, the modified CD11b⁺CD45⁺ cell is a CD11b⁺CD45⁺CD14⁺ cell. In certain embodiments, the modified CD11b⁺CD45⁺CD14⁺ cell is a human CD11b⁺CD45⁺CD14⁺ cell. In certain embodiments, the modified CD11b⁺CD45⁺CD14⁺ cell is a mammalian CD11b⁺CD45⁺CD14⁺ cell. In certain embodiments, the modified CD11b⁺CD45⁺CD14⁺ cell is a primate CD11b⁺CD45⁺CD14⁺ cell. In certain embodiments, the modified CD11b⁺CD45⁺CD14⁺ cell is a non-human primate CD11b⁺CD45⁺CD14⁺ cell. In certain embodiments, the modified CD11b⁺CD45⁺CD14⁺ cell is a rodent, e.g., mouse, CD11b⁺CD45⁺CD14⁺ cell.

In some aspects, provided herein is a homogeneous population of modified CD11b⁺CD45⁺ cells comprising a polynucleotide or a polypeptide encoding a chimeric antigen receptor (CAR) (such as a CAR described in Section 5.3) and genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into (a) a SIRPA gene; (b) a CISH gene; and/or (c) a SIGLEC10 gene, and the modified CD11b⁺CD45⁺ cells comprise genetic disruption of the gene(s) in which the polynucleotide encoding the CAR is integrated. In certain embodiments, a polynucleotide encoding the CAR (such as a polynucleotide described in Section 5.4) is integrated into (a) a B2M gene; (b) a CIITA gene; and/or (c) an RFX gene, and the modified CD11b⁺CD45⁺ cells comprise genetic disruption of the gene(s) in which the polynucleotide encoding the CAR is integrated. In certain embodiments, a polynucleotide encoding the CAR (such as a polynucleotide described in Section 5.4) is integrated into a safe harbor locus, e.g., an AAVS1 locus.

In certain embodiments, a polynucleotide encoding the CAR (such as a polynucleotide described in Section 5.4) is integrated into a modified CD11b⁺CD45⁺ cell using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In some embodiments, the homogeneous population of modified CD11b⁺CD45⁺ cells comprises genetic disruption of the SIRPA gene. In some embodiments, the modified CD11b⁺CD45⁺ cells are heterozygous for the genetic disruption of the SIRPA gene. In some embodiments, the modified CD11b⁺CD45⁺ cells are homozygous for the genetic disruption of the SIRPA gene. In certain embodiments, the homogeneous population of modified CD11b⁺CD45⁺ cells are isogenic.

In some embodiments, the homogeneous population of modified CD11b⁺CD45⁺ cells comprises genetic disruption of the SIGLEC10 gene. In some embodiments, the modified CD11b⁺CD45⁺ cells are heterozygous for the genetic disruption of the SIGLEC10 gene. In some embodiments, the modified CD11b⁺CD45⁺ cells are homozygous for the genetic disruption of the SIGLEC10 gene. In certain embodiments, the homogeneous population of modified CD11b⁺CD45⁺ cells are isogenic.

In some embodiments, the homogeneous population of modified CD11b⁺CD45⁺ cells comprises genetic disruption of the CISH gene. In some embodiments, the modified CD11b⁺CD45⁺ cells are heterozygous for the genetic disruption of the CISH gene. In some embodiments, the modified CD11b⁺CD45⁺ cells are homozygous for the genetic disruption of the CISH gene. In certain embodiments, the homogeneous population of modified CD11b⁺CD45⁺ cells are isogenic.

In specific embodiments, the homogeneous population of modified CD11b⁺CD45⁺ cells comprises genetic disruption of SIRPA and CISH. In certain embodiments, the homogeneous population of modified CD11b⁺CD45⁺ cells comprises genetic disruption of SIRPA and SIGLEC10. In further embodiments, the homogeneous population of modified CD11b⁺CD45⁺ cells comprises genetic disruption of SIGLEC10 and CISH. In still further embodiments, the homogeneous population of modified CD11b⁺CD45⁺ cells comprises genetic disruption of SIGLEC10, SIRPA, and CISH. In certain embodiments, the homogeneous population of modified CD11b⁺CD45⁺ cells are isogenic.

In some embodiments, the population of modified CD11b⁺CD45⁺ cells are derived from induced pluripotent stem cells (iPSCs). In certain embodiments, the iPSCs have been reprogrammed from a cell selected from the group consisting of a peripheral blood mononuclear cell (PBMC), CD34⁺ cord blood, a macrophage, a monocyte, and a fibroblast.

In some embodiments, the iPSCs have been reprogrammed from a PBMC. In some embodiments, the iPSCs have been reprogrammed from CD34⁺ cord blood. In some embodiments, the iPSCs have been reprogrammed from a macrophage. In some embodiments, the iPSCs are reprogrammed from a monocyte. In some embodiments, the iPSCs have been reprogrammed from a fibroblast. In some embodiments, the iPSC is a TC-1133 iPSC.

In certain embodiments, the modified CD11b⁺CD45⁺ cell further comprises (i) genetic disruption of a B2M gene, (ii) genetic disruption of a CIITA gene, (iii) genetic disruption of an RFX gene, and/or (iv) an exogenous polynucleotide encoding HLA-E. In certain embodiments, the modified CD11b⁺CD45⁺ cell provided herein further comprises genetic disruption of (i) a B2M gene, a CIITA gene and/or an RFX gene, and (ii) an exogenous polynucleotide encoding HLA-E. In certain embodiments, the modified CD11b⁺CD45⁺ cell provided herein further comprises genetic disruption of a B2M gene and a CIITA gene (e.g., B2M/CIITA double knockout), and an exogenous polynucleotide encoding HLA-E. In certain embodiments, the modified CD11b⁺CD45⁺ cell provided herein further comprises genetic disruption of a B2M gene, a CIITA gene, and an RFX gene, and an exogenous polynucleotide encoding HLA-E.

In certain embodiments, the modified CD11b⁺CD45⁺ cell provided herein is homozygous for the genetic disruption of the B2M gene, CIITA gene, and/or RFX gene. In certain embodiments, the modified CD11b⁺CD45⁺ cell provided herein is heterozygous for the genetic disruption of the B2M gene, CIITA gene, and/or RFX gene.

In some embodiments, the population of modified CD11b⁺CD45⁺ cells comprise an agent capable of preventing or reducing interaction between CD47 and SIRPα (see, e.g., WO 2019/241403, incorporated by reference in its entirety). In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody.

In some embodiments, the population of modified CD11b⁺CD45⁺ cells comprises an agent capable of preventing or reducing interaction between CD24 and SIGLEC10 (see, e.g., WO 2019/241403, incorporated by reference in its entirety). In some embodiments, the agent comprises an anti-CD24 antibody. In some embodiments, the agent comprises an anti-SIGLEC10 antibody. In some embodiments, the agent comprises a soluble SIGLEC10 polypeptide.

The present disclosure further provides a population comprising a modified CD11b⁺CD45⁺ cell disclosed herein. For example, the present disclosure further provides a population of cells comprising a modified CD11b⁺CD45⁺ cell disclosed herein wherein at least about 80%, at least about 85%, at least about 90%, at least about 95%, at least about 96%, at least about 97%, at least about 98%, at least about 99%, at least about 99.5%, at least about 99.8%, from about 80% to about 95%, from about 85% to about 95%, from about 90% to about 95%, from about 90% to about 100%, from about 95% to about 98%, from about 95% to about 99%, or from about 95% to 100% of the cells in the population are the modified CD11b⁺CD45⁺ cells.

### 5.2. Methods of Generating Modified Cells

In certain aspects, the present disclosure relates to modified cells comprising a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. Various techniques known to one of skill in the art or described herein may be used to disrupt a gene or expression of the corresponding protein encoded by the gene. Exemplary techniques include genome editing tools, such as TALEN (Transcription Activator-Like Effector Nucleases), Zinc-finger nucleases (ZFN), and CRISPR, as well as variations thereof (e.g., base editing, prime editing, etc.); RNA interference (RNAi) (e.g., shRNA, or siRNA), or Cre/LoxP-based conditional knockout. In certain embodiments, genetic disruption deletes expression of the corresponding protein encoded by the gene. In some embodiments, genetic disruption prevents or reduces expression of the full-length protein encoded by the gene.

In some embodiments, the gene editing system comprises a CRISPR system. In some embodiments, the CRISPR system comprises a Class 2 CRISPR system. Class 2 systems currently represent a single protein that is categorized into three distinct types (types II, V and VI). Any class 2 CRISPR system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (e.g., Cpf1, MAD7), Cas12b (C2c1), Cas12c (C2c3), Cas12d (CasY), Cas12e (CasX), Cas12f, Cas12g, Cas12h, Cas12i and Cas12k (C2c5). Exemplary Class 2 Type VI systems include Cas13, Cas13a (C2c2) Cas13b, Cas13c and Cas13d.

The endonuclease protein (e.g., nucleic acid-directed nuclease) may be derived from any bacterial or archaeal Cas protein. Any suitable CRISPR system is contemplated as within the scope of the instant disclosure. In some embodiments, the endonuclease protein comprises one or more of Cas1, Cas1B, Cas2, Cas3, Cas4, Cas5, Cas6, Cas7, Cas8, Cas9, Cas10, Cas12, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the endonuclease protein is a Cas9 protein, a Cpf1 protein, a C2c1 protein, a C2c2 protein, a C2c3 protein, Cas3, Cas3-HD, Cas5, Cas7, Cas8, Cas10, Cas12, modified versions thereof, or combinations or complexes of these.

In some embodiments, the Class 2 CRISPR system comprises a type V system. In some embodiments, the Type V system comprises a Cas12a CRISPR/Cas protein. Exemplary Cas12a proteins include MAD7, isolated from Eubacterium rectale. MAD7 uses T-rich protospacer adjacent motifs (PAMs) such as YTTN. Exemplary MAD7 proteins are described in US20190360001 and WO 2021119563, the contents of which are incorporated by reference herein in their entireties.

In some embodiments, the modified cells provided herein comprise genetic disruption of SIRPA (e.g., Homo sapiens NCBI Gene ID: 140885, Mus musculus NCBI Gene ID: 19261). SIRPA encodes for a SIRPα polypeptide, an immunoglobulin-like cell surface receptor for CD47. In certain embodiments, genetic disruption of SIRPA prevents or reduces expression of a SIRPα polypeptide capable of interaction with CD47. In certain embodiments, genetic disruption of SIRPA prevents a SIRPα polypeptide from being capable of interaction with CD47. In some embodiments, the SIRPA gene is a human SIRPA gene. In some embodiments, the SIRPA gene is a non-human SIRPA gene (e.g., a mouse SIRPA gene).

In some embodiments, genetic disruption of SIRPA is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of SIRPA is performed using gRNA. In certain embodiments, genetic disruption of SIRPA is performed using gRNA and a Cas12a protein. In certain embodiments, genetic disruption is performed using gRNA and a MAD7 protein. In some embodiments, the gRNA comprises a targeting sequence complementary to a SIRPA target sequence. In some embodiments, the SIRPA target sequence comprises coding sequence, for example SIRPA mRNA sequence. In some embodiments, the gRNA targets exon 1 of SIRPA. In some embodiments, the gRNA targets exon 2 of SIRPA. For example, exon 2 of SIRPA can be targeted with a guide RNA (gRNA), such as a guide RNA sequence 5′-CGACCUCUCUGAUCCCUGUG-3′ (SEQ ID NO:63) that targets the genetic sequence 5′-TGACCTCCCTGATCCCTGTG-3′ (SEQ ID NO:64). In some embodiments, the gRNA targets exon 3 of SIRPA. In some embodiments, the gRNA targets exon 4 of SIRPA. In some embodiments, the gRNA targets exon 5 of SIRPA. In some embodiments, the gRNA targets exon 6 of SIRPA. In some embodiments, the gRNA targets exon 7 of SIRPA. In some embodiments, the gRNA targets exon 8 of SIRPA. In some embodiments, the gRNA targets exon 9 of SIRPA. In some embodiments, the gRNA targets exon 10 of SIRPA. In some embodiments, the gRNA targets exon 11 of SIRPA. In some embodiments, the gRNA targets exon 12 of SIRPA. In some embodiments, the SIRPA target sequence comprises non-coding sequence. Exemplary non-coding sequence includes SIRPA intronic, promoter, 5′ untranslated region (UTR), 3′ UTR, or enhancer sequence. In some embodiments, genetic disruption of SIRPA is performed by RNAi. In some embodiments, the genetic disruption of SIRPA is performed by conditional knockout. In some embodiments, the genetic disruption of SIRPA is performed in human cells.

In some embodiments, the modified cells provided herein comprise genetic disruption of CISH (e.g., Homo sapiens NCBI Gene ID: 1154, Mus musculus NCBI Gene ID: 12700). CISH is an inhibitory immune checkpoint gene. In certain embodiments, genetic disruption of CISH prevents or reduces expression of a CISH polypeptide. In some embodiments, the CISH gene is a human CISH gene. In some embodiments, the CISH gene is a non-human CISH gene (e.g., a mouse CISH gene).

In some embodiments, genetic disruption of CISH is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of CISH is performed using gRNA. In some embodiments, the gRNA comprises a targeting sequence complementary to a CISH target sequence. In some embodiments, the CISH target sequence comprises coding sequence, for example CISH mRNA sequence. In some embodiments, the gRNA targets exon 1 of CISH. In some embodiments, the gRNA targets exon 2 of CISH. In some embodiments, the gRNA targets exon 3 of CISH. In some embodiments, the gRNA targets exon 4 of CISH. In some embodiments, the CISH target sequence comprises non-coding sequence. Exemplary non-coding sequence includes CISH intronic, promoter, 5′ untranslated region (UTR), 3′ UTR, or enhancer sequence. In some embodiments the gRNA is compatible with a gRNA/Cas9 system. In some embodiments the gRNA is compatible with a gRNA/Cas12a (e.g., MAD7) system. In some embodiments, genetic disruption of CISH is performed by RNAi. In some embodiments, genetic disruption of CISH is performed by conditional knockout. Non-limiting examples of CISH inhibitors, including CISH guide RNAs (gRNAs), include those provided in WO 2017/100861, WO 2017/023803, WO 2018/075664, WO 2019/213610, and WO 2019/217956, each of which are incorporated by reference in its entirety. In some embodiments, the genetic disruption of CISH is performed in human cells.

In some embodiments, the modified cells provided herein comprise genetic disruption of SIGLEC10 (e.g., Homo sapiens NCBI Gene ID: 89790, Mus musculus NCBI Gene ID: 243958). SIGLEC10 is a ligand for CD52, vascular adhesion protein 1 (VAP-1), and CD24. The CD24-SIGLEC10 interaction can serve as an anti-phagocytic signal (see, e.g., Barkal A A, et al., Nature. 2019 August; 572(7769):392-396). Thus, in some embodiments genetic disruption of SIGLEC10 prevents or reduces expression of a SIGLEC10 polypeptide capable of interaction with CD24 on a target cell. In some embodiments genetic disruption of SIGLEC10 prevents a SIGLEC10 polypeptide from being capable of interaction with CD24 on a target cell. In some embodiments, the SIGLEC10 gene is a human SIGLEC10 gene. In some embodiments, the SIGLEC10 gene is a non-human SIGLEC10 gene (e.g., a mouse SIGLEC10 gene).

In some embodiments, genetic disruption of SIGLEC10 is performed by a genome editing tool, such as TALEN, ZFN, or CRISPR (e.g., by deletion of one or more exons, introduction of a stop codon, introduction of a null mutation or inactivation of the promoter). In some embodiments, genetic disruption of SIGLEC10 is performed using gRNA. In some embodiments, the gRNA comprises a targeting sequence complementary to a SIGLEC10 target sequence. In some embodiments, the SIGLEC10 target sequence comprises coding sequence, for example SIGLEC10 mRNA sequence. In some embodiments, the gRNA targets exon 1 of SIGLEC10. In some embodiments, the gRNA targets exon 2 of SIGLEC10. In some embodiments, the gRNA targets exon 3 of SIGLEC10. In some embodiments, the gRNA targets exon 4 of SIGLEC10. In some embodiments, the gRNA targets exon 5 of SIGLEC10. In some embodiments, the gRNA targets exon 6 of SIGLEC10. In some embodiments, the gRNA targets exon 7 of SIGLEC10. In some embodiments, the gRNA targets exon 8 of SIGLEC10. In some embodiments, the gRNA targets exon 9 of SIGLEC10. In some embodiments, the gRNA targets exon 10 of SIGLEC10. In some embodiments, the gRNA targets exon 11 of SIGLEC10. In some embodiments, the SIGLEC10 target sequence comprises non-coding sequence. Exemplary non-coding sequence includes SIGLEC10 intronic, promoter, 5′ untranslated region (UTR), 3′ UTR, or enhancer sequence. In some embodiments, genetic disruption of SIGLEC10 is performed by RNAi. In some embodiments, genetic disruption of SIGLEC10 is performed by conditional knockout. In some embodiments, the genetic disruption of SIGLEC10 is performed in human cells.

In specific embodiments, the modified cells provided herein comprise genetic disruption of SIRPA and CISH. In certain embodiments, the modified cells provided herein comprise genetic disruption of SIRPA and SIGLEC10. In further embodiments, the modified cells provided herein comprise genetic disruption of SIGLEC10 and CISH. In still further embodiments, the modified cells comprise genetic disruption of SIGLEC10, SIRPA, and CISH.

In certain embodiments, the methods provided herein further comprise selecting a modified cell that comprises expression of a CAR. In some embodiments, the methods provided herein comprise selecting a modified cell that comprises expression of a CAR, and an undetectable level of nonspecific genetic modifications. In some embodiments, the methods provided herein comprise selecting a modified cell that comprises genetic disruption of SIGLEC10, SIRPA, and/or CISH. In some embodiments, the methods provided herein comprise selecting a modified cell that comprises genetic disruption of SIGLEC10, SIRPA, and/or CISH, and an undetectable level of nonspecific genetic modifications. In some embodiments, the methods provided herein comprise selecting a modified cell that comprises (a) genetic disruption of SIGLEC10, SIRPA, and/or CISH; and (b) expression of a CAR. In some embodiments, the methods provided herein comprise selecting a modified cell that comprises (a) genetic disruption of SIGLEC10, SIRPA, and/or CISH; (b) expression of a CAR, and (c) an undetectable level of nonspecific genetic modifications.

### 5.2.1. Methods of Generating Modified Pluripotent Cells

In one aspect, provided herein is a method of generating a modified pluripotent cell, comprising: genetically disrupting in a pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene. In some embodiments, the modified pluripotent cell comprises genetic disruption of the SIGLEC10 gene. In some embodiments, the modified pluripotent cell further comprises genetic disruption of the SIRPA gene and/or the CISH gene. For example, in certain embodiments, the modified pluripotent cell comprises genetic disruption of SIGLEC10 and SIRPA. In other embodiments, the modified pluripotent cell comprises genetic disruption of SIGLEC10 and CISH. In some embodiments, the modified pluripotent cell comprises genetic disruption of SIRPA and CISH. In still further embodiments, the modified pluripotent cell comprises genetic disruption of SIGLEC10, SIRPA, and CISH. In some embodiments, the modified pluripotent cell is heterozygous for the genetic disruption of the SIGLEC10 gene. In some embodiments, the modified pluripotent cell is homozygous for the genetic disruption of the SIGLEC10 gene. In some embodiments, the modified pluripotent cell is heterozygous for the genetic disruption of the SIRPA gene. In some embodiments, the modified pluripotent cell is homozygous for the genetic disruption of the SIRPA gene. In some embodiments, the modified pluripotent cell is heterozygous for the genetic disruption of the CISH gene. In some embodiments, the modified pluripotent cell is homozygous for the genetic disruption of the CISH gene.

In one aspect, provided herein is a method of generating a modified pluripotent cell, comprising: (a) obtaining a pluripotent cell expressing a (i) a chimeric antigen receptor (“CAR”) (such as a CAR described in Section 5.3), (ii) a polynucleotide encoding a CAR (such as a polynucleotide described in Section 5.4) (iii) a vector comprising a polynucleotide encoding a CAR (such as a vector described in Section 5.5), or (iv) a CAR polypeptide (such as a polypeptide described in Section 5.6); and (b) genetically disrupting in the pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene. In some embodiments, the method comprises genetically disrupting the SIRPA gene. In certain embodiments, the modified pluripotent cell is heterozygous for the genetic disruption of the SIRPA gene. In certain embodiments, the modified pluripotent cell is homozygous for the genetic disruption of the SIRPA gene. In some embodiments, the method comprises genetically disrupting the SIGLEC10 gene. In certain embodiments, the modified pluripotent cell is heterozygous for the genetic disruption of the SIGLEC10 gene. In certain embodiments, the modified pluripotent cell is homozygous for the genetic disruption of the SIGLEC10 gene. In some embodiments, the method comprises genetically disrupting the CISH gene. In certain embodiments, the modified pluripotent cell is heterozygous for the genetic disruption of the CISH gene. In certain embodiments, the modified pluripotent cell is homozygous for the genetic disruption of the CISH gene. In certain embodiments, the modified pluripotent cell is homozygous for the genetic disruption of the CISH gene. In specific embodiments, the method comprises genetically disrupting SIRPA and CISH. In certain embodiments, the method comprises genetically disrupting SIRPA and SIGLEC10. In further embodiments, the method comprises genetically disrupting SIGLEC10 and CISH. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

In another aspect, provided herein is a method of generating a modified pluripotent cell, comprising introducing (i) a polynucleotide encoding a CAR (such as a polynucleotide described in Section 5.4), (ii) a vector comprising a polynucleotide encoding a CAR (such as a vector described in Section 5.5), or (iii) a CAR polypeptide (such as a polypeptide described in Section 5.6) into a pluripotent cell comprising genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the pluripotent cell comprises genetic disruption of the SIRPA gene. In certain embodiments, the pluripotent cell is heterozygous for the genetic disruption of the SIRPA gene. In certain embodiments, the pluripotent cell is homozygous for the genetic disruption of the SIRPA gene. In some embodiments, the pluripotent cell comprises genetic disruption of the SIGLEC10 gene. In certain embodiments, the modified pluripotent cell is heterozygous for the genetic disruption of the SIGLEC10 gene. In certain embodiments, the modified pluripotent cell is homozygous for the genetic disruption of the SIGLEC10 gene. In some embodiments, the pluripotent cell comprises genetic disruption of the CISH gene. In certain embodiments, the modified pluripotent cell is heterozygous for the genetic disruption of the CISH gene. In certain embodiments, the modified pluripotent cell is homozygous for the genetic disruption of the CISH gene. In specific embodiments, the modified pluripotent cell comprises genetic disruption of SIRPA and CISH. In certain embodiments, the modified pluripotent cell comprises genetic disruption of SIRPA and SIGLEC10. In further embodiments, the modified pluripotent cell comprises genetic disruption of SIGLEC10 and CISH. In still further embodiments, the modified pluripotent cell comprises genetic disruption of SIGLEC10, SIRPA, and CISH. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, a polynucleotide encoding the CAR (such as a polynucleotide described in Section 5.4) is integrated into a safe harbor locus, e.g., an AAVS1 locus.

In yet another aspect, provided herein is a method of generating a modified pluripotent cell, comprising: (a) introducing (i) a polynucleotide encoding a CAR (such as a polynucleotide described in Section 5.4), (ii) a vector comprising a polynucleotide encoding a CAR (such as a vector described in Section 5.5), or (iii) a CAR polypeptide (such as a polypeptide described in Section 5.6) into a pluripotent cell; and (b) genetically disrupting in the pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene. In some embodiments, the method comprises genetically disrupting the SIRPA gene. In certain embodiments, the modified pluripotent cell is heterozygous for the genetic disruption of the SIRPA gene. In certain embodiments, the modified pluripotent cell is homozygous for the genetic disruption of the SIRPA gene. In some embodiments, the method comprises genetically disrupting the SIGLEC10 gene. In certain embodiments, the modified pluripotent cell is heterozygous for the genetic disruption of the SIGLEC10 gene. In certain embodiments, the modified pluripotent cell is homozygous for the genetic disruption of the SIGLEC10 gene. In some embodiments, the method comprises genetically disrupting the CISH gene. In certain embodiments, the modified pluripotent cell is heterozygous for the genetic disruption of the CISH gene. In certain embodiments, the modified pluripotent cell is homozygous for the genetic disruption of the CISH gene. In specific embodiments, the method comprises genetically disrupting SIRPA and CISH. In certain embodiments, the method comprises genetically disrupting SIRPA and SIGLEC10. In further embodiments, the method comprises genetically disrupting SIGLEC10 and CISH. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, the method comprises integrating a polynucleotide encoding the CAR (such as a polynucleotide described in Section 5.4) into a target gene selected from the group consisting of a SIRPA gene, a CISH gene, and a SIGLEC10 gene, such that the target gene(s) is genetically disrupted. In certain embodiments, the method comprises integrating a polynucleotide encoding the CAR (such as a polynucleotide described in Section 5.4) into a target gene selected from the group consisting of a B2M gene, a CIITA gene, and an RFX gene, such that the target gene(s) is genetically disrupted. In certain embodiments, the method comprises integrating a polynucleotide encoding the CAR (such as a polynucleotide described in Section 5.4) into a safe harbor locus, e.g., an AAVS1 locus.

In a further aspect, provided herein is a method of generating a modified pluripotent cell, comprising: (a) genetically disrupting in a pluripotent cell: (i) a signal regulatory protein alpha (SIRPA) gene; (ii) a cytokine inducible SH2 containing protein (CISH) gene; and/or (iii) a sialic acid-binding Ig-like lectin 10 (SIGLEC10) gene; and (b) introducing (i) a polynucleotide encoding a CAR (such as a polynucleotide described in Section 5.4), (ii) a vector comprising a polynucleotide encoding a CAR (such as a vector described in Section 5.5), or (iii) a CAR polypeptide (such as a polypeptide described in Section 5.6) into the pluripotent cell. In some embodiments, the method comprises genetically disrupting the SIRPA gene. In certain embodiments, the modified pluripotent cell is heterozygous for the genetic disruption of the SIRPA gene. In certain embodiments, the modified pluripotent cell is homozygous for the genetic disruption of the SIRPA gene. In some embodiments, the method comprises genetically disrupting the SIGLEC10 gene. In certain embodiments, the modified pluripotent cell is heterozygous for the genetic disruption of the SIGLEC10 gene. In certain embodiments, the modified pluripotent cell is homozygous for the genetic disruption of the SIGLEC10 gene. In some embodiments, the method comprises genetically disrupting the CISH gene. In certain embodiments, the modified pluripotent cell is heterozygous for the genetic disruption of the CISH gene. In certain embodiments, the modified pluripotent cell is homozygous for the genetic disruption of the CISH gene. In certain embodiments, the modified pluripotent cell is homozygous for the genetic disruption of the CISH gene. In specific embodiments, the method comprises genetically disrupting SIRPA and CISH. In certain embodiments, the method comprises genetically disrupting SIRPA and SIGLEC10. In further embodiments, the method comprises genetically disrupting SIGLEC10 and CISH. In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, the method comprises integrating a polynucleotide encoding the CAR (such as a polynucleotide described in Section 5.4) into a target gene selected from the group consisting of a SIRPA gene, a CISH gene, and a SIGLEC10 gene, such that the target gene(s) is genetically disrupted. In certain embodiments, the method comprises integrating a polynucleotide encoding the CAR (such as a polynucleotide described in Section 5.4) into a target gene selected from the group consisting of a B2M gene, a CIITA gene, and an RFX gene, such that the target gene(s) is genetically disrupted. In certain embodiments, the method comprises integrating a polynucleotide encoding the CAR (such as a polynucleotide described in Section 5.4) into a safe harbor locus, e.g., an AAVS1 locus.

In certain embodiments, a polynucleotide encoding the CAR (such as a polynucleotide described in Section 5.4) is integrated into the pluripotent cell using the SLEEK technology. In certain embodiments, the polynucleotide encoding the CAR is integrated in frame with and downstream (3′) of a coding sequence of an essential gene, and wherein at least part of the essential gene comprises an exogenous coding sequence. In certain embodiments, the correct knock-in cells would retain essential gene function while also integrating the polynucleotide encoding the CAR. Cells with non-productive insertions and deletions would undergo negative selection. In certain embodiments, the essential gene encodes a gene product that is required for survival and/or proliferation of the cell. In certain embodiments, the essential gene is a housekeeping gene. In certain embodiments, the essential gene encodes glyceraldehyde 3-phosphate dehydrogenase (GAPDH). In certain embodiments, the essential gene is an essential gene as disclosed in International Patent Publication WO2022235811.

In certain embodiments, the methods provided herein further comprise selecting a modified pluripotent cell that comprises expression of a CAR. In some embodiments, the methods provided herein comprise selecting a modified pluripotent cell that comprises expression of a CAR, and an undetectable level of nonspecific genetic modifications. In some embodiments, the methods provided herein comprise selecting a modified pluripotent cell that comprises genetic disruption of SIGLEC10, SIRPA, and/or CISH. In some embodiments, the methods provided herein comprise selecting a modified pluripotent cell that comprises genetic disruption of SIGLEC10, SIRPA, and/or CISH, and an undetectable level of nonspecific genetic modifications. In some embodiments, the methods provided herein comprise selecting a modified pluripotent cell that comprises (a) genetic disruption of SIGLEC10, SIRPA, and/or CISH; and (b) expression of a CAR. In some embodiments, the methods provided herein comprise selecting a modified pluripotent cell that comprises (a) genetic disruption of SIGLEC10, SIRPA, and/or CISH; (b) expression of a CAR, and (c) an undetectable level of nonspecific genetic modifications.

In certain embodiments, the method disclosed herein further comprises obtaining a pluripotent cell comprising (i) genetic disruption of a B2M gene, (ii) genetic disruption of a CIITA gene, (iii) genetic disruption of an RFX gene, and/or (iv) an exogenous polynucleotide encoding HLA-E.

In certain embodiments, the method disclosed herein further comprises (i) genetically disrupting in the pluripotent cell a B2M gene, a CIITA gene, and/or an RFX gene, and/or (ii) introducing an exogenous polynucleotide encoding a HLA-E.

In certain embodiments, the modified pluripotent cell provided herein is homozygous for the genetic disruption of the B2M gene, CIITA gene, and/or RFX gene. In certain embodiments, the modified pluripotent cell provided herein is heterozygous for the genetic disruption of the B2M gene, CIITA gene, and/or RFX gene.

Pluripotent cells can give rise to a multiplicity of cell types, and include, for example, induced pluripotent stem cells (iPSCs). In certain embodiments, a pluripotent stem cell as described herein is a mammalian pluripotent cell, e.g., a mammalian iPSC. In certain embodiments, a pluripotent stem cell as described herein is a human pluripotent cell, e.g., a human iPSC. In certain embodiments, a pluripotent cell as described herein is a multipotent cell, e.g., a human multipotent cell. In certain embodiments, a pluripotent cell as described herein is a hematopoietic stem cell (HSC), e.g., a human HSC. In certain embodiments, a pluripotent cell as described herein is an embryonic stem cell (ESC), for example, a human embryonic stem cell. In certain non-limiting embodiments, a pluripotent cell as described herein is a parthenogenic stem cell, e.g., a human parthenogenetic stem cell, a primordial germ cell-like pluripotent stem cell, e.g., a human primordial germ cell-like pluripotent stem cell, an epiblast stem cell, e.g., a human epiblast stem cell, an F-class pluripotent stem cell, e.g., a human F-class pluripotent stem cell, a somatic stem cell, e.g., a human somatic stem cell, or any other cell, e.g., human cell, capable of lineage specific differentiation. In certain embodiments, a pluripotent cell described herein is a non-human pluripotent cell. In certain embodiments, a pluripotent cell as described herein is a nonhuman primate pluripotent cell. In certain embodiments, a pluripotent cell as described herein is a rodent, e.g., mouse, pluripotent cell.

In certain embodiments, a pluripotent cell as described herein is not an embryonic stem cell, for example, is not a human embryonic stem cell. In certain embodiments, a pluripotent cell as described herein is not capable of differentiating or developing into all cell types. For example, in certain embodiments, a human pluripotent cell as described herein is not capable of differentiating or developing into all cell types of the human body.

In certain embodiments, the pluripotent cell provided herein is a TC-1133 cell. In certain embodiments, the pluripotent cell provided herein is a hematopoietic stem cell, e.g., a human hematopoietic stem cell. In certain embodiments, a hematopoietic stem cell is produced from a modified iPSC as described herein.

Accordingly, in some embodiments, the modified pluripotent cell provided herein is an iPSC. In certain embodiments, the pluripotent cell provided herein is a human induced pluripotent stem cell (hiPSC). In certain embodiments, the iPSC has been reprogrammed from a cell selected from the group consisting of a peripheral blood mononuclear cell (PBMC), CD34⁺ cord blood, a macrophage, a monocyte, and a fibroblast. In some embodiments, the iPSC has been reprogrammed from a PBMC. In some embodiments, the iPSC has been reprogrammed from CD34⁺ cord blood. In some embodiments, the iPSC has been reprogrammed from a macrophage. In some embodiments, the iPSC has been reprogrammed from a monocyte. In some embodiments, the iPSC has been reprogrammed from a fibroblast.

### 5.2.2. Methods of Generating Modified Myeloid Progenitor Cells

Also provided herein is a method of generating a homogeneous population of modified myeloid progenitor cells (such as the modified myeloid progenitor cells described in Section 5.1.2), comprising expanding and differentiating a modified pluripotent cell (such as the modified pluripotent cells described in Section 5.1.1) under conditions sufficient for cell differentiation into a population of myeloid progenitor cells. In another aspect, provided herein is a method of generating a homogeneous population of modified myeloid progenitor cells (such as the modified myeloid progenitor cells described in Section 5.1.2), comprising differentiating a modified pluripotent cell (such as the modified pluripotent cells described in Section 5.1.1) under conditions sufficient for cell differentiation into a population of myeloid progenitor cells. In some embodiments, the modified pluripotent cell provided herein is an iPSC. Techniques known to one of skill in the art or described herein (such as in Section 5.8) may be used to differentiate a pluripotent cell into a myeloid progenitor cell. In some embodiments, the iPSC is a TC-1133 iPSC.

In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2). In some embodiments, 100% of the cells express a myeloid progenitor cell marker, such as for example Lin⁻, CD34⁺, CD38⁺, and CD45RA⁻, or any other myeloid progenitor cell marker(s) known to one of skill in the art or described herein (such as in Section 5.10.2).

### 5.2.3. Methods of Generating Modified Monocytes

Also provided herein is a method of generating a homogeneous population of modified monocytes (such as the modified monocytes described in Section 5.1.3) comprising expanding and differentiating a modified pluripotent cell (such as the modified pluripotent cells described in Section 5.1.1) under conditions sufficient for cell differentiation into a population of monocytes. In another aspect, provided herein is a method of generating a homogeneous population of modified monocytes (such as the modified monocytes described in Section 5.1.3) comprising differentiating a modified pluripotent cell (such as the modified pluripotent cells described in Section 5.1.1) under conditions sufficient for cell differentiation into a monocyte. In a further aspect, provided herein is a method of generating a homogeneous population of modified monocytes (such as the modified monocytes described in Section 5.1.3) comprising expanding and differentiating a modified myeloid progenitor cell (such as the modified myeloid progenitor cell described in Section 5.1.2) under conditions sufficient for cell differentiation into a population of monocytes. In yet another aspect, provided herein is a method of generating a homogeneous population of modified monocytes (such as the modified monocytes described in Section 5.1.3) comprising differentiating a modified myeloid progenitor cell (such as the modified myeloid progenitor cell described in Section 5.1.2) under conditions sufficient for cell differentiation into a monocyte. In some embodiments, the modified pluripotent cell provided herein is an iPSC. Techniques known to one of skill in the art or described herein (such as in Section 5.8) may be used to differentiate a pluripotent cell into a monocyte or a myeloid progenitor cell into a monocyte.

In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a monocyte marker, such as for example CD11b⁺CD45⁺, CD14⁺CD11b⁺CD45⁺, or CD14 CD11b⁺CD45⁺, or any other monocyte marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a monocyte marker, such as for example CD11b⁺CD45⁺, CD14⁺CD11b⁺CD45⁺, or CD14⁻CD11b⁺CD45⁺, or any other monocyte marker(s) known to one of skill in the art or described herein (such as in Section 5.10.2). In some embodiments, 100% of the cells express a monocyte marker, such as for example CD11b⁺CD45⁺, CD14⁺CD11b⁺CD45⁺, or CD14⁻CD11b⁺CD45⁺, or any other monocyte marker(s) known to one of skill in the art or described herein (such as in Section 5.10.2).

### 5.2.4. Methods of Generating Modified Macrophages

Also provided herein is a method of generating a homogeneous population of modified macrophages (such as the modified macrophages described in Section 5.1.4) comprising expanding and differentiating a modified pluripotent cell (such as the modified pluripotent cells described in Section 5.1.1) under conditions sufficient for cell differentiation into a population of macrophages. In another aspect, provided herein is a method of generating a homogeneous population of modified macrophages (such as the modified macrophages described in Section 5.1.4) comprising differentiating a modified pluripotent cell (such as the modified pluripotent cells described in Section 5.1.1) under conditions sufficient for cell differentiation into a macrophage. In a further aspect, provided herein is a method of generating a homogeneous population of modified macrophages (such as the modified macrophages described in Section 5.1.4) comprising expanding and differentiating a modified myeloid progenitor cell (such as the modified myeloid progenitor cell described in Section 5.1.2) under conditions sufficient for cell differentiation into a population of macrophages. In yet another aspect, provided herein is a method of generating a homogeneous population of modified macrophages (such as the modified macrophages described in Section 5.1.4) comprising differentiating a modified myeloid progenitor cell (such as the modified myeloid progenitor cell described in Section 5.1.2) under conditions sufficient for cell differentiation into a macrophage. In a further aspect, provided herein is a method of generating a homogeneous population of modified macrophages (such as the modified macrophages described in Section 5.1.4) comprising expanding and differentiating a modified monocyte (such as the modified monocyte described in Section 5.1.3) under conditions sufficient for cell differentiation into a population of macrophages. In yet another aspect, provided herein is a method of generating a homogeneous population of modified macrophages (such as the modified macrophages described in Section 5.1.4) comprising differentiating a modified monocyte (such as the modified monocyte described in Section 5.1.3) under conditions sufficient for cell differentiation into a macrophage. In some embodiments, the modified pluripotent cell provided herein is an iPSC. Techniques known to one of skill in the art or described herein (such as in Section 5.8) may be used to differentiate a pluripotent cell into a macrophage, a myeloid progenitor cell into a macrophage, or a monocyte into a macrophage.

The present disclosure further provides a population of cells comprising a modified macrophage as described herein. In some embodiments, at least about 80%, at least about 90%, at least about 95%, at least about 96%, at least about 97%, at least about 98%, at least about 99%, at least about 99.5%, at least about 99.8%, from about 80% to about 95%, from about 85% to about 95%, about 90% to about 95%, about 90% to about 100%, about 95% to about 98%, about 95% to about 99%, or about 95% to 100% of the population expresses a macrophage marker, such as for example CD11b⁺CD45⁺, CD14⁺CD11b⁺CD45⁺, or CD14⁻CD11b⁺CD45⁺, or any other macrophage marker(s) known to one of skill in the art or described herein (such as in Section 5.10.2). In some embodiments, greater than about 90%, greater than about 95%, greater than about 96%, greater than about 97%, greater than about 98%, or greater than about 99% of the population expresses a macrophage marker, such as for example CD11b⁺CD45⁺, CD14⁺CD11b⁺CD45⁺, or CD14⁻CD11b⁺CD45⁺, or any other macrophage marker(s) known to one of skill in the art or described herein (such as in Section 5.10.2). In some embodiments, 100% of the cells express a macrophage marker, such as for example CD11b⁺CD45⁺, CD14⁺CD11b⁺CD45⁺, or CD14⁻CD11b⁺CD45⁺, or any other macrophage marker(s) known to one of skill in the art or described herein (such as in Section 5.10.2).

### 5.2.5. Methods of Generating Modified CD11b⁺CD45⁺ Cells

Also provided herein is a method of generating a homogenous population of modified CD11b⁺CD45⁺ cells or a cell population comprising a modified CD11b⁺CD45⁺ cells disclosed herein (such as the cell populations disclosed described in Section 5.1.5), comprising expanding and differentiating a modified pluripotent cell (such as the modified pluripotent cells described in Section 5.1.1) under conditions sufficient for cell differentiation into a population of CD11b⁺CD45⁺ cells (e.g., the differentiation methods disclosed in Section 5.8). In another aspect, provided herein is a method of generating a modified CD11b⁺CD45⁺ cell (such as the modified CD11b⁺CD45⁺ cell described in Section 5.1.5), a homogenous population of modified CD11b⁺CD45⁺ cells or a cell population comprising a modified CD11b⁺CD45⁺ cell disclosed herein (such as the cell populations disclosed described in Section 5.1.5), comprising differentiating a modified pluripotent cell (such as the modified pluripotent cells described in Section 5.1.1) under conditions sufficient for cell differentiation into a CD11b⁺CD45⁺ cell (e.g., the differentiation methods disclosed in Section 5.8). Techniques known to one of skill in the art or described herein (such as in Section 5.8) may be used to differentiate a pluripotent cell into a CD11b⁺CD45⁺ cell.

The present disclosure also provides a method of generating a modified CD11b⁺CD45⁺ cell, e.g., a modified CD11b⁺CD45⁺CD14⁺ cell, comprising genetically disrupting in a CD11b⁺CD45⁺ cell, e.g., a modified CD11b⁺CD45⁺CD14⁺ cell, (a) a SIRPA gene; (b) a CISH gene; and/or (c) a SIGLEC10 gene.

In certain embodiments, the modified CD11b⁺CD45⁺ cell, e.g., modified CD11b⁺CD45⁺CD14⁺ cell, comprises genetic disruption of the SIGLEC10 gene. In some embodiments, the modified CD11b⁺CD45⁺ cell, e.g., modified CD11b⁺CD45⁺CD14⁺ cell, further comprises genetic disruption of the SIRPA gene and/or the CISH gene. For example, in certain embodiments, the modified CD11b⁺CD45⁺ cell, e.g., modified CD11b⁺CD45⁺CD14⁺ cell, comprises genetic disruption of SIGLEC10 and SIRPA. In other embodiments, the modified CD11b⁺CD45⁺ cell, e.g., modified CD11b⁺CD45⁺CD14⁺ cell, comprises genetic disruption of SIGLEC10 and CISH. In some embodiments, the modified CD11b⁺CD45⁺ cell, e.g., modified CD11b⁺CD45⁺CD14⁺ cell, comprises genetic disruption of SIRPA and CISH. In still further embodiments, the modified CD11b⁺CD45⁺ cell, e.g., modified CD11b⁺CD45⁺CD14⁺ cell, comprises genetic disruption of SIGLEC10, SIRPA, and CISH. In some embodiments, the modified CD11b⁺CD45⁺ cell, e.g., modified CD11b⁺CD45⁺CD14⁺ cell, is heterozygous for the genetic disruption of the SIGLEC10 gene. In some embodiments the modified CD11b⁺CD45⁺ cell, e.g., modified CD11b⁺CD45⁺CD14⁺ cell, is homozygous for the genetic disruption of the SIGLEC10 gene. In some embodiments, the modified CD11b⁺CD45⁺ cell, e.g., modified CD11b⁺CD45⁺CD14⁺ cell, is heterozygous for the genetic disruption of the SIRPA gene. In some embodiments, the modified CD11b⁺CD45⁺ cell, e.g., modified CD11b⁺CD45⁺CD14⁺ cell, is homozygous for the genetic disruption of the SIRPA gene. In some embodiments, the modified CD11b⁺CD45⁺ cell, e.g., modified CD11b⁺CD45⁺CD14⁺ cell, is heterozygous for the genetic disruption of the CISH gene. In some embodiments, the modified CD11b⁺CD45⁺ cell, e.g., modified CD11b⁺CD45⁺CD14⁺ cell, is homozygous for the genetic disruption of the CISH gene.

The present disclosure also provides a method of generating a modified CD11b⁺CD45⁺ cell, e.g., a human CD11b⁺CD45⁺ cell, comprising introducing a polynucleotide encoding a CAR (such as a CAR described in Section 5.3). In some embodiments, the CAR comprises a non-lymphoid intracellular signaling domain. In certain embodiments, the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1. In certain embodiments, the modified CD11b⁺CD45⁺ cell comprises genetic disruption of (a) a SIRPA gene; (b) a CISH gene; and/or (c) a SIGLEC10 gene.

### 5.3. Chimeric Antigen Receptors

As provided herein, the present disclosure relates to chimeric antigen receptors (CARs). In some embodiments, the CAR activates monocyte/macrophage activity. In some embodiments, a CAR provided herein comprises an antigen recognition moiety (such as an antigen recognition moiety described in Section 5.3.1), a hinge domain (such as a hinge domain described in Section 5.3.2), a transmembrane domain (such as a transmembrane domain described in Section 5.3.3), and an intracellular domain (such as an intracellular domain described in Section 5.3.4).

### 5.3.1. Antigen Recognition Moiety

In some aspects, a chimeric antigen receptor (CAR) of the present disclosure comprises an antigen recognition moiety that recognizes and binds to a specific binding element on the target of interest. Non-limiting examples of the antigen recognition moiety include a single-chain variable fragment (scFv), nanobodies, ligands to cognate receptors, native receptors against targets, and small peptides.

Various antigens suitable for targeting with a CAR are known in the art, and each are suitable for use with the present disclosure. In some embodiments, the antigen recognition domain recognizes an antigen selected from the group consisting of HER2/neu, PSMA, Claudin18, CD20, CD5, BCMA, TAC1, GD2, mesothelin and CD19. In some embodiments, the antigen recognition domain recognizes HER2/neu. In some embodiments, the antigen recognition domain recognizes PSMA. In some embodiments, the antigen recognition domain recognizes Claudin 18. In some embodiments, the antigen recognition domain recognizes CD20. In some embodiments, the antigen recognition domain recognizes CD20. In some embodiments, the antigen recognition domain recognizes CD5. In some embodiments, the antigen recognition domain recognizes BCMA. In some embodiments, the antigen recognition domain recognizes CD19. In some embodiments, the antigen recognition domain recognizes mesothelin. In some embodiments, the antigen recognition domain recognizes TAC1. In some embodiments, the antigen recognition domain recognizes GD2. In certain aspects, the antigen recognition moiety is a single-chain variable fragment (scFv).

### 5.3.2. Hinge Domain

In certain aspects, provided herein is a chimeric antigen receptor (“CAR”) comprising a hinge domain. A hinge domain (also referred to as a spacer) is a structure between the antigen recognition moiety and the cell membrane. Non-limiting examples include, for example, a hinge domain derived from an IgG subclass (such as IgG1 and IgG4), IgD, CD28, CSF1R, a Fcγ receptor, and CD8 domains. In some embodiments, provided herein is a hinge domain lacking FcγR binding activity. In some embodiments, provided herein is a hinge domain derived from a native T cell molecule (e.g., CD28, or CD8). In some embodiments, a CAR provided herein comprises a hinge domain derived from CD8. In some embodiments, a CAR provided herein comprises a hinge domain derived from an IgG subclass. In specific embodiments, a CAR provided herein comprises a hinge domain derived from IgG1. In specific embodiments, a CAR provided herein comprises a hinge domain derived from IgG2. In specific embodiments, a CAR provided herein comprises a hinge domain derived from IgG3. In specific embodiments, a CAR provided herein comprises a hinge domain derived from IgG4. In some embodiments, a CAR provided herein comprises a hinge domain derived from IgD.

### 5.3.3. Transmembrane Domain

In certain aspects, provided herein is a CAR comprising a transmembrane domain. A transmembrane domain is a structure that facilitates anchoring the CAR in a cell membrane, and generally consists of a hydrophobic α-helix that spans the cell membrane. Non-limiting examples include, for example, a transmembrane domain derived from an IgG subclass (such as IgG1 and IgG4), IgD, CD28, CSF1R, a Fcγ receptor, and CD8 domains. In some embodiments, the transmembrane domain is a single-span transmembrane (e.g., a transmembrane domain derived from CD4, CD8α, or CD28). In some embodiments, a CAR provided herein comprises a transmembrane domain derived from CD8. In some embodiments, a CAR provided herein does not comprise a transmembrane domain derived from CD8. In some embodiments, a CAR provided herein comprises a transmembrane domain derived from CD28. In some embodiments, a CAR provided herein comprises a transmembrane domain derived from CSF1R. In some embodiments, a CAR provided herein does not comprise a transmembrane domain derived from CD28. In some embodiments, a CAR provided herein comprises a transmembrane domain derived from CD86. In some embodiments, a CAR provided herein does not comprise a transmembrane domain derived from CD86. In some embodiments, a CAR provided herein comprises a transmembrane domain derived from TLR4. In some embodiments, a CAR provided herein does not comprise a transmembrane domain derived from TLR4. In some embodiments, the transmembrane domain is derived from the same source as at least one of the intracellular domains. For example, the transmembrane domain can be derived from CD86 and at least one intracellular domain can also be derived from CD86. As a further example, the transmembrane can be derived from DECTIN-1 and at least one intracellular domain can also be derived from DECTIN-1.

### 5.3.4. Intracellular Domain

In certain aspects, provided herein is a CAR comprising one or more intracellular domains. An intracellular domain transmits activation signals. In some embodiments, an intracellular domain provided herein comprises a non-lymphoid intracellular signaling domain (such as a non-lymphoid intracellular signaling domain described in Section 5.3.4.1). In some embodiments, an intracellular domain provided herein comprises a non-lymphoid intracellular signaling domain (such as a non-lymphoid intracellular signaling domain described in Section 5.3.4.1) and an intracellular domain comprising one or more immune-receptor-tyrosine-based-activation-motif (ITAM) (such as an ITAM containing intracellular signaling domain described in Section 5.3.4.2).

### 5.3.4.1. Non-Lymphoid Intracellular Signaling Domain

As provided herein, the present disclosure is based, in part, on the discovery that a CAR comprising a non-lymphoid intracellular signaling domain can enhance the activity of a macrophage, relative to an otherwise identical CAR having an intracellular domain consisting of a CD3ζ intracellular signaling domain. Accordingly, in specific embodiments, provided herein is a CAR comprising an intracellular domain that does not consist of a CD3ζ intracellular signaling domain. For example, in some embodiments, the intracellular signaling domain comprises a non-lymphoid intracellular signaling domain and a CD32 intracellular signaling domain. In other specific embodiments, the CAR comprises at least one intracellular domain that does not include an intracellular signaling domain derived from (a) the native B cell receptor complex (e.g. CD20, CD22, CD79a, CD79b), or (b) the native T cell receptor complex (e.g., CD3ζ, CD3δ, or CD3ε). In further embodiments, the CAR comprises at least one intracellular domain that does not include an intracellular signaling domain involved in the classical T cell signaling pathway, such as a co-stimulatory molecule from the CD28 family (e.g., CD28 or ICOS) or tumor necrosis factor receptor (TNFR) family (e.g., 4-1BB/CD137, OX40, or CD27).

Exemplary non-lymphoid intracellular signaling domains are provided in Table 3, below. In some embodiments, the non-lymphoid intracellular signaling domain is selected from the group consisting of BAI-1, CD86/B7-2, Lox1c, TIM4, MEGF10, SCARF1, CD93, DAP12, SLAMF7, IFNγR2, 2B4/CD244, DECTIN-1, CD206, DECTIN-3, CLEC2, CD40, and CD80/B7-1. In some embodiments, the non-lymphoid intracellular signaling domain is selected from the group consisting of CD86, Lox1c, 2B4, DECTIN-1, and CD40. In some embodiments, the non-lymphoid intracellular signaling domain is selected from the group consisting of CD80 and CD86.

TABLE 3
Exemplary Non-Lymphoid Intracellular Signaling Domains

| Intracellular | | SEQ |
|---|---|---|
| Signaling | | ID |
| Domain | Sequence | NO |
| CD86 | KWKKKKRPRNSYKCGTNTMEREESEQTKKREKIHIPERSDEAQRVFKS | 1 |
| | SKTSSCDKSDTCF | |
| LOX1 | CWWPSYLFQLGKAKKGNSKEDPQDKVTQIKLDDFTM | 2 |
| 2B4 | WRRKRKEKQSETSPKEFLTIYEDVKDLKTRRNHEQEQTFPGGGSTIYS | 3 |
| | MIQSQSSAPTS | |
| DECTIN-1 | RWPPSAACSGKESVVAIRTNSQSDFHLQTYGDEDLNELDPHYEM | 4 |
| DECTIN-2 | LRLSLWGRKETSQPQQEQMM | 5 |
| TIM1 | KKYFFKKEVQQLSVSFSSLQIKALQNAVEKEVQAEDNIYIENSLYATD | 6 |
| CD40 | KKVAKKPTNKAPHPKQEPQEINFPDDLPGSNTAAPVQETLHGCQPVTQ | 7 |
| | EDGKESRISVQERQ | |
| CD11b | KLGFFKRQYKDMMSEGGPPGAEPQ | 8 |
| CSF1R | KYKQKPKYQVRWKIIESYEGNSYTFIDPTQLPYNEKWEFPRNNLQFGK | 9 |
| | TLGAGAFGKVVEATAFGLGKEDAVLKVAVKMLKSTAHADEKEALMSEL | |
| | KIMSHLGQHENIVNLLGACTHGGPVLVITEYCCYGDLLNFLRRKAEAM | |
| | LGPSLSPGQDPEGGVDYKNIHLEKKYVRRDSGFSSQGVDTYVEMRPVS | |
| | TSSNDSFSEQDLDKEDGRPLELRDLLHFSSQVAQGMAFLASKNCIHRD | |
| | VAARNVLLTNGHVAKIGDFGLARDIMNDSNYIVKGNARLPVKWMAPES | |
| | IFDCVYTVQSDVWSYGILLWEIFSLGLNPYPGILVNSKFYKLVKDGYQ | |
| | MAQPAFAPKNIYSIMQACWALEPTHRPTFQQICSFLQEQAQEDRRERD | |
| | YTNLPSSSRSGGSGSSSSELEEESSSEHLTCCEQGDIAQPLLQPNNYQ | |
| | FC | |
| CSF2R | KRFLRIQRLFPPVPQIKDKLNDNHEVEDEIIWEEFTPEEGKGYREEVL | 10 |
| | TVKEIT | |
| CSF3R | SPNRKNPLWPSVPDPAHSSLGSWVPTIMEEDAFQLPGLGTPPITKLTV | 11 |
| | LEEDEKKPVPWESHNSSETCGLPTLVQTYVLQGDPRAVSTQPQSQSGT | |
| | SDQVLYGQLLGSPTSPGPGHYLRCDSTQPLLAGLTPSPKSYENLWFQA | |
| | SPLGTLVTPAPSQEDDCVFGPLLNFPLLQGIRVHGMEALGSF | |
| IL1R | KIFKIDIVLWYRDSCYDFLPIKASDGKTYDAYILYPKTVGEGSTSDCD | 12 |
| | IFVFKVLPEVLEKQCGYKLFIYGRDDYVGEDIVEVINENVKKSRRLII | |
| | ILVRETSGFSWLGGSSEEQIAMYNALVQDGIKVVLLELEKIQDYEKMP | |
| | ESIKFIKQKHGAIRWSGDFTQGPQSAKTRFWKNVRYHMPVQRRSPSSK | |
| | HQLLSPATKEKLQREAHVPLG | |
| TLR1 | SYLDLPWYLRMVCQWTQTRRRARNIPLEELQRNLQFHAFISYSGHDSF | 13 |
| | WVKNELLPNLEKEGMQICLHERNFVPGKSIVENIITCIEKSYKSIFVL | |
| | SPNFVQSEWCHYELYFAHHNLFHEGSNSLILILLEPIPQYSIPSSYHK | |
| | LKSLMARRTYLEWPKEKSKRGLFWANLRAAINIKLTEQAKK | |
| TLR2 | HRFHGLWYMKMMWAWLQAKRKPRKAPSRNICYDAFVSYSERDAYWVEN | 14 |
| | LMVQELENFNPPFKLCLHKRDFIPGKWIIDNIIDSIEKSHKTVFVLSE | |
| | NFVKSEWCKYELDFSHFRLFDENNDAAILILLEPIEKKAIPQRFCKLR | |
| | KIMNTKTYLEWPMDEAQREGFWVNLRAAIKS | |
| TLR2 | CHRFHGLWYMKMMWAWLQAKRKPRKAPSRNICYDAFVSYSERDAYWVE | 15 |
| | NLMVQELENFNPPFKLCLHKRDFIPGKWIIDNIIDSIEKSHKTVFVLS | |
| | ENFVKSEWCKYELDFSHFRLFDENNDAAILILLEPIEKKAIPQRFCKL | |
| | RKIMNTKTYLEWPMDEAQREGFWVNLRAAIKS | |
| TLR3 | FEGWRISFYWNVSVHRVLGFKEIDRQTEQFEYAAYIIHAYKDKDWVWE | 16 |
| | HFSSMEKEDQSLKFCLEERDFEAGVFELEAIVNSIKRSRKIIFVITHH | |
| | LLKDPLCKRFKVHHAVQQAIEQNLDSIILVFLEEIPDYKLNHALCLRR | |
| | GMFKSHCILNWPVQKERIGAFRHKLQVALGSKNSVH | |
| TLR4 | KFYFHLMLLAGCIKYGRGENIYDAFVIYSSQDEDWVRNELVKNLEEGV | 17 |
| | PPFQLCLHYRDFIPGVAIAANIIHEGFHKSRKVIVVVSQHFIQSRWCI | |
| | FEYEIAQTWQFLSSRAGIIFIVLQKVEKTLLRQQVELYRLLSRNTYLE | |
| | WEDSVLGRHIFWRRLRKALLDGKSWNPEGTVGTGCNWQEATSI | |
| TLR5 | TKFRGFCFICYKTAQRLVFKDHPQGTEPDMYKYDAYLCFSSKDFTWVQ | 18 |
| | NALLKHLDTQYSDQNRFNLCFEERDFVPGENRIANIQDAIWNSRKIVC | |
| | LVSRHFLRDGWCLEAFSYAQGRCLSDLNSALIMVVVGSLSQYQLMKHQ | |
| | SIRGFVQKQQYLRWPEDFQDVGWFLHKLSQQILKKEKEKKKDNNIPLQ | |
| | TVATIS | |
| TLR6 | YLDLPWYLRMVCQWTQTRRRARNIPLEELQRNLQFHAFISYSEHDSAW | 19 |
| | VKSELVPYLEKEDIQICLHERNFVPGKSIVENIINCIEKSYKSIFVLS | |
| | PNFVQSEWCHYELYFAHHNLFHEGSNNLILILLEPIPQNSIPNKYHKL | |
| | KALMTQRTYLQWPKEKSKRGLFWANIRAAFNMKLTLVTENNDVKS | |
| TLR7 | HLYFWDVWYIYHFCKAKIKGYQRLISPDCCYDAFIVYDTKDPAVTEWV | 20 |
| | LAELVAKLEDPREKHFNLCLEERDWLPGQPVLENLSQSIQLSKKTVFV | |
| | MTDKYAKTENFKIAFYLSHQRLMDEKVDVIILIFLEKPFQKSKFLQLR | |
| | KRLCGSSVLEWPTNPQAHPYFWQCLKNALATDNHVAYSQVFKETV | |
| TLR8 | AHHLFYWDVWFIYNVCLAKVKGYRSLSTSQTFYDAYISYDTKDASVTD | 21 |
| | WVINELRYHLEESRDKNVLLCLEERDWDPGLAIIDNLMQSINQSKKTV | |
| | FVLTKKYAKSWNFKTAFYLALQRLMDENMDVIIFILLEPVLQHSQYLR | |
| | LRQRICKSSILQWPDNPKAEGLFWQTLRNVVLTENDSRYNNMYVDSIK | |
| | QY | |
| SCARF1 | CCCWAPRSDLKDRPARDGATVSRMKLQVWGTLTSLGSTLPCRSLSSHK | 22 |
| | LPWVTVSHHDPEVPFNHSFIEPPSAGWATDDSFSSDPESGEADEVPAY | |
| | CVPPQEGMVPVAQAGSSEASLAAGAFPPPEDASTPFAIPRTSSLARAK | |
| | RPSVSFAEGTKFAPQSRRSSGELSSPLRKPKRLSRGAQSGPEGREAEE | |
| | STGPEEAEAPESFPAAASPGDSATGHRRPPLGGRTVAEHVEAIEGSVQ | |
| | ESSGPVTTIYMLAGKPRGSEGPVRSVFRHFGSFQKGQAEAKVKRAIPK | |
| | PPRQALNRKKGSPGLASGSVGQSPNSAPKAGLPGATGPMAVRPEEAVR | |
| | GLGAGTESSRRAQEPVSGCGSPEQDPQKQAEEERQEEPEYENVVPISR | |
| | PPEP | |
| SCARF2 | CCCACRGKDPTRRPRPRRELSLGRKKAPHRLCGRFSRISMKLPRIPLR | 23 |
| | RQKLPKVVVAHHDLDNTLNCSFLEPPSGLEQPSPSWSSRASFSSFDTT | |
| | DEGPVYCVPHEEAPAESRDPEVPTVPAEAPAPSPVPLTTPASAEEAIP | |
| | LPASSDSERSASSVEGPGGALYARVARREARPARARGEIGGLSLSPSP | |
| | ERRKPPPPDPATKPKVSWIHGKHSAAAAGRAPSPPPPGSEAAPSPSKR | |
| | KRTPSDKSAHTVEHGSPRTRDPTPRPPGLPEEATALAAPSPPRARARG | |
| | RGPGLLEPTDAGGPPRSAPEAASMLAAELRGKTRSLGRAEVALGAQGP | |
| | REKPAPPQKAKRSVPPASPARAPPATETPGPEKAATDLPAPETPRKKT | |
| | PIQKPPRKKSREAAGELGRAGAPTL | |
| DC-SIGN | CGALSKYGRTQRFGLGRLQEEELLGLQQLRPEKSDSM | 24 |
| CXCL16 | CKRRRGQSPQSSPDLPVHYIPVAPDSNT | 25 |
| CD93 | GLLVYRKRRAKREEKKEKKPQNAADSYSWVPERAESRAMENQYSPTPG | 26 |
| | TDC | |
| LIMPII | GQGSMDEGTADERAPLIRT | 27 |
| MELLEC | LAVPRWTSSPARHETRRPEPHRTTASGQSHLSMTTDGDDDLMDRTSSY | 28 |
| | KAQM | |
| DNGR1 | LCCAGSCKNSSLCKQYTDPAPSDWQLSTYIEEEHM | 29 |
| TIM4 | LRGKLMETYCSQKHTRLDYIGDSKNVLNDVQHGREDEDGLFTL | 30 |
| FCER1G | LRKRRDSLSLSTQRTQGPAESARNLEYVSVSPTNNTVYASVTHSNRET | 31 |
| | EIWTPRENDTITIYSTINHSKESKPTFSRATALDNV | |
| VEGFR2 | LRTVKRANGGELKTGYLSIVMDPDELPLDEHCERLPYDASKWEFPRDR | 32 |
| | LKLGKPLGRGAFGQVIEADAFGIDKTATCRTVAVKMLKEGATHSEHRA | |
| | LMSELKILIHIGHHLNVVNLLGACTKPGGPLMVIVEFCKFGNLSTYLR | |
| | SKRNEFVPYKTKGARFRQGKDYVGAIPVDLKRRLDSITSSQSSASSGF | |
| | VEEKSLSDVEEEEAPEDLYKDFLTLEHLICYSFQVAKGMEFLASRKCI | |
| | HRDLAARNILLSEKNVVKICDFGLARDIYKDPDYVRKGDARLPLKWMA | |
| | PETIFDRVYTIQSDVWSFGVLLWEIFSLGASPYPGVKIDEEFCRRLKE | |
| | GTRMRAPDYTTPEMYQTMLDCWHGEPSQRPTFSELVEHLGNLLQANAQ | |
| | QDGKDYIVLPISETLSMEEDSGLSLPTSPVSCMEEEEVCDPKFHYDNT | |
| | AGISQYLQNSKRKSRPVSVKTFEDIPLEEPEVKVIPDDNQTDSGMVLA | |
| | SEELKTLEDRTKLSPSFGGMVPSKSRESVASEGSNQTSGYQSGYHSDD | |
| | TDTTVYSSEEAELLKLIEIGVQTGSTAQILQPDSGTTLSSPPV | |
| IFNγR2 | LVLKYRGLIKYWFHTPPSIPLQIEEYLKDPTQPILEALDKDSSPKDDV | 33 |
| | WDSVSIISFPEKEQEDVLQTL | |
| CLEC2 | MVRWWSSSASGVSILAPKRTKINLTIYGDEDQM | 34 |
| MARCO | NVGNRRKPKPVNIEFPEMAIQHFAAQQTESLLEDEKLIKKNRM | 35 |
| DEC-205 | QRHRLHLAGFSSVRYAQGVNEDEIMLPSFHD | 36 |
| RAGE | QRRQRRGEERKAPENQEEEEERAELNQSEEPEAGESSTGGP | 37 |
| STAB1 | RARGKPMGFGFSAFQAEDDADDDFSPWQEGTNPTLVSVPNPVFGSDTF | 38 |
| | CEPFDDSLLEEDFPDTQRILTVK | |
| CD28 | RSKRSRLLHSDYMNMTPRRPGPTRKHYQPYAPPRDFAAYRS | 39 |
| LANGERIN | RVTPTKGPVLSPGSKPPPERPWLSINQKDVTFHADPAEKEVTM | 40 |
| CD36 | SYCACRSKTIK | 41 |
| CD80 | TTYCFAPRCRERRRNERLRRESVRPV | 42 |
| CD80 | TYCFAPRCRERRRNERLRRESVRPV | 43 |
| SLAMF7 | WFLKRERQEEYIEEKKRVDICRETPNICPHSGENTEYDTIPHTNRTIL | 44 |
| | KEDPANTVYSTVEIPKKMENPHSLLTMPDTPRLFAYENVI | |
| DAP12 | YFLGRLVPRGRGAAEAATRKQRITETESPYQELQGQRSDVYSDLNTQR | 45 |
| | PYYK | |
| MRC1/CD206 | YKKRRVHLPQEGAFENTLYFNSQSSPGTSDMKDLVGNIEQNEHSVI | 46 |
| MEGF10 | YRHKQKGKESSMPAVTYTPAMRVVNADYTISGTLPHSNGGNANSHYFT | 47 |
| | NPSYHTLTQCATSPHVNNRDRMTVTKSKNNQLFVNLKNVNPGKRGPVG | |
| | DCTGTLPADWKHGGYLNELGAFGLDRSYMGKSLKDLGKNSEYNSSNCS | |
| | LSSSENPYATIKDPPVLIPKSSECGYVEMKSPARRDSPYAEINNSTSA | |
| | NRNVYEVEPTVSVVQGVFSNNGRLSQDPYDLPKNSHIPCHYDLLPVRD | |
| | SSSSPKQEDSGGSSSNSSSSSE | |
| STABILIN2 | YSYFRINRRTIGFQHFESEEDINVAALGKQQPENISNPLYESTTSAPP | 48 |
| | EPSYDPFTDSEERQLEGNDPLRTL | |
| BAI-1 | YWSMRLQARGGPSPLKSNSDSARLPISSGSTSSSRI | 49 |

In specific embodiments, a CAR provided herein comprises an intracellular signaling domain comprising an amino acid sequence at least 90%, at least 95%, or 100% identical to an amino acid sequence provided in Table 3. In specific embodiments, a CAR provided herein comprises an intracellular signaling domain comprising an amino acid sequence at least 90%, at least 95%, or 100% identical to SEQ ID NO:1, SEQ ID NO:2, SEQ ID NO:3, or SEQ ID NO:4. In some embodiments, a CAR provided herein comprises an intracellular signaling domain comprising an amino acid sequence at least 90%, at least 95%, or 100% identical to SEQ ID NO:1. In some embodiments, a CAR provided herein comprises an intracellular signaling domain comprising an amino acid sequence at least 90%, at least 95%, or 100% identical to SEQ ID NO:2. In some embodiments, a CAR provided herein comprises an intracellular signaling domain comprising an amino acid sequence at least 90%, at least 95%, or 100% identical to SEQ ID NO:3. In some embodiments, a CAR provided herein comprises an intracellular signaling domain comprising an amino acid sequence at least 90%, at least 95%, or 100% identical to SEQ ID NO:4. In some embodiments, a CAR provided herein comprises an intracellular signaling domain comprising an amino acid sequence at least 90%, at least 95%, or 100% identical to SEQ ID NO:5. In some embodiments, a CAR provided herein comprises an intracellular signaling domain comprising an amino acid sequence at least 90%, at least 95%, or 100% identical to SEQ ID NO:6. In some embodiments, a CAR provided herein comprises an intracellular signaling domain comprising an amino acid sequence at least 90%, at least 95%, or 100% identical to SEQ ID NO:7. In some embodiments, a CAR provided herein comprises an intracellular signaling domain comprising an amino acid sequence at least 90%, at least 95%, or 100% identical to SEQ ID NO:8. In some embodiments, a CAR provided herein comprises an intracellular signaling domain comprising an amino acid sequence at least 90%, at least 95%, or 100% identical to SEQ ID NO:9. In some embodiments, a CAR provided herein comprises an intracellular signaling domain comprising an amino acid sequence at least 90%, at least 95%, or 100% identical to SEQ ID NO:10. In some embodiments, a CAR provided herein comprises an intracellular signaling domain comprising an amino acid sequence at least 90%, at least 95%, or 100% identical to SEQ ID NO:11. In some embodiments, a CAR provided herein comprises an intracellular signaling domain comprising an amino acid sequence at least 90%, at least 95%, or 100% identical to SEQ ID NO:12. In some embodiments, a CAR provided herein comprises an intracellular signaling domain comprising an amino acid sequence at least 90%, at least 95%, or 100% identical to SEQ ID NO:13. In some embodiments, a CAR provided herein comprises an intracellular signaling domain comprising an amino acid sequence at least 90%, at least 95%, or 100% identical to SEQ ID NO:14. In some embodiments, a CAR provided herein comprises an intracellular signaling domain comprising an amino acid sequence at least 90%, at least 95%, or 100% identical to SEQ ID NO:15. In some embodiments, a CAR provided herein comprises an intracellular signaling domain comprising an amino acid sequence at least 90%, at least 95%, or 100% identical to SEQ ID NO:16. In some embodiments, a CAR provided herein comprises an intracellular signaling domain comprising an amino acid sequence at least 90%, at least 95%, or 100% identical to SEQ ID NO:17. In some embodiments, a CAR provided herein comprises an intracellular signaling domain comprising an amino acid sequence at least 90%, at least 95%, or 100% identical to SEQ ID NO:18. In some embodiments, a CAR provided herein comprises an intracellular signaling domain comprising an amino acid sequence at least 90%, at least 95%, or 100% identical to SEQ ID NO:19. In some embodiments, a CAR provided herein comprises an intracellular signaling domain comprising an amino acid sequence at least 90%, at least 95%, or 100% identical to SEQ ID NO:20. In some embodiments, a CAR provided herein comprises an intracellular signaling domain comprising an amino acid sequence at least 90%, at least 95%, or 100% identical to SEQ ID NO:21. In some embodiments, a CAR provided herein comprises an intracellular signaling domain comprising an amino acid sequence at least 90%, at least 95%, or 100% identical to SEQ ID NO:22. In some embodiments, a CAR provided herein comprises an intracellular signaling domain comprising an amino acid sequence at least 90%, at least 95%, or 100% identical to SEQ ID NO:23. In some embodiments, a CAR provided herein comprises an intracellular signaling domain comprising an amino acid sequence at least 90%, at least 95%, or 100% identical to SEQ ID NO:24. In some embodiments, a CAR provided herein comprises an intracellular signaling domain comprising an amino acid sequence at least 90%, at least 95%, or 100% identical to SEQ ID NO:25. In some embodiments, a CAR provided herein comprises an intracellular signaling domain comprising an amino acid sequence at least 90%, at least 95%, or 100% identical to SEQ ID NO:26. In some embodiments, a CAR provided herein comprises an intracellular signaling domain comprising an amino acid sequence at least 90%, at least 95%, or 100% identical to SEQ ID NO:27. In some embodiments, a CAR provided herein comprises an intracellular signaling domain comprising an amino acid sequence at least 90%, at least 95%, or 100% identical to SEQ ID NO:28. In some embodiments, a CAR provided herein comprises an intracellular signaling domain comprising an amino acid sequence at least 90%, at least 95%, or 100% identical to SEQ ID NO:29. In some embodiments, a CAR provided herein comprises an intracellular signaling domain comprising an amino acid sequence at least 90%, at least 95%, or 100% identical to SEQ ID NO:30. In some embodiments, a CAR provided herein comprises an intracellular signaling domain comprising an amino acid sequence at least 90%, at least 95%, or 100% identical to SEQ ID NO:31. In some embodiments, a CAR provided herein comprises an intracellular signaling domain comprising an amino acid sequence at least 90%, at least 95%, or 100% identical to SEQ ID NO:32. In some embodiments, a CAR provided herein comprises an intracellular signaling domain comprising an amino acid sequence at least 90%, at least 95%, or 100% identical to SEQ ID NO:33. In some embodiments, a CAR provided herein comprises an intracellular signaling domain comprising an amino acid sequence at least 90%, at least 95%, or 100% identical to SEQ ID NO:34. In some embodiments, a CAR provided herein comprises an intracellular signaling domain comprising an amino acid sequence at least 90%, at least 95%, or 100% identical to SEQ ID NO:35. In some embodiments, a CAR provided herein comprises an intracellular signaling domain comprising an amino acid sequence at least 90%, at least 95%, or 100% identical to SEQ ID NO:36. In some embodiments, a CAR provided herein comprises an intracellular signaling domain comprising an amino acid sequence at least 90%, at least 95%, or 100% identical to SEQ ID NO:37. In some embodiments, a CAR provided herein comprises an intracellular signaling domain comprising an amino acid sequence at least 90%, at least 95%, or 100% identical to SEQ ID NO:38. In some embodiments, a CAR provided herein comprises an intracellular signaling domain comprising an amino acid sequence at least 90%, at least 95%, or 100% identical to SEQ ID NO:39. In some embodiments, a CAR provided herein comprises an intracellular signaling domain comprising an amino acid sequence at least 90%, at least 95%, or 100% identical to SEQ ID NO:40. In some embodiments, a CAR provided herein comprises an intracellular signaling domain comprising an amino acid sequence at least 90%, at least 95%, or 100% identical to SEQ ID NO:41. In some embodiments, a CAR provided herein comprises an intracellular signaling domain comprising an amino acid sequence at least 90%, at least 95%, or 100% identical to SEQ ID NO:42. In some embodiments, a CAR provided herein comprises an intracellular signaling domain comprising an amino acid sequence at least 90%, at least 95%, or 100% identical to SEQ ID NO:43. In some embodiments, a CAR provided herein comprises an intracellular signaling domain comprising an amino acid sequence at least 90%, at least 95%, or 100% identical to SEQ ID NO:44. In some embodiments, a CAR provided herein comprises an intracellular signaling domain comprising an amino acid sequence at least 90%, at least 95%, or 100% identical to SEQ ID NO:45. In some embodiments, a CAR provided herein comprises an intracellular signaling domain comprising an amino acid sequence at least 90%, at least 95%, or 100% identical to SEQ ID NO:46. In some embodiments, a CAR provided herein comprises an intracellular signaling domain comprising an amino acid sequence at least 90%, at least 95%, or 100% identical to SEQ ID NO:47. In some embodiments, a CAR provided herein comprises an intracellular signaling domain comprising an amino acid sequence at least 90%, at least 95%, or 100% identical to SEQ ID NO:48. In some embodiments, a CAR provided herein comprises an intracellular signaling domain comprising an amino acid sequence at least 90%, at least 95%, or 100% identical to SEQ ID NO:49. In some embodiments, a CAR provided herein comprises an intracellular signaling domain comprising an amino acid sequence at least 90%, at least 95%, or 100% identical to SEQ ID NO:42 or SEQ ID NO:43.

### 5.3.4.2. ITAM-Containing Intracellular Signaling Domain

In some aspects, the present disclosure relates, in part, to a CAR having at least two intracellular signaling domains (e.g., a first intracellular signaling domain, such as a non-lymphoid intracellular signaling domain described in Section 5.3.4.1, and a second intracellular signaling domain comprising one or more immune-receptor-tyrosine-based-activation-motifs (ITAMs)). Exemplary ITAM-containing intracellular signaling domains are provided in Table 4.

In specific embodiments, a CAR provided herein comprises an intracellular signaling domain comprising an amino acid sequence at least 90%, at least 95%, or 100% identical to an amino acid sequence provided in Table 4. In specific embodiments, a CAR provided herein comprises an intracellular signaling domain comprising or consisting of an amino acid sequence at least 90%, at least 95%, or 100% identical to SEQ ID NO:50, or SEQ ID NO:53. In some embodiments, a CAR provided herein comprises or consists of an intracellular signaling domain comprising an amino acid sequence at least 90%, at least 95%, or 100% identical to SEQ ID NO:50. In some embodiments, a CAR provided herein comprises or consists of an intracellular signaling domain comprising an amino acid sequence at least 90%, at least 95%, or 100% identical to SEQ ID NO:51. In some embodiments, a CAR provided herein comprises or consists of an intracellular signaling domain comprising an amino acid sequence at least 90%, at least 95%, or 100% identical to SEQ ID NO:52. In some embodiments, a CAR provided herein comprises or consists of an intracellular signaling domain comprising an amino acid sequence at least 90%, at least 95%, or 100% identical to SEQ ID NO:53.

TABLE 4
Exemplary ITAM Containing
Intracellular Signaling Domains

| Intra- | | |
|---|---|---|
| cellular | | SEQ |
| Signaling | | ID |
| Domain | Sequence | NO |
| CD3ζ | RVKFSRSADAPAYQQGQNQLYNE | 50 |
| | LNLGRREEYDVLDKRRGRDPEMG | |
| | GKPQRRKNPQEGLYNELQKDKMA | |
| | EAYSEIGMKGERRRGKGHDGLYQ | |
| | GLSTATKDTYDALHMQALPPR | |
| CD3ϵ | KNRKAKAKPVTRGAGAGGRQRGQ | 51 |
| | NKERPPPVPNPDYEPIRKGQRDL | |
| | YSGLNQRRI | |
| CD3δ | GHETGRLSGAADTQALLRNDQVY | 52 |
| | QPLRDRDDAQYSHLGGNWARNK | |
| FcγRI | RKELKRKKKWDLEISLDSGHEKK | 53 |
| | VISSLQEDRHLEEELKCQEQKEE | |
| | QLQEGVHRKEPQGAT | |

In some embodiments, a CAR provided herein comprises an intracellular domain having a CD86 intracellular signaling domain and a CD3ζ intracellular signaling domain. In specific embodiments, a CAR provided herein comprises an intracellular domain having a CD86 intracellular signaling domain comprising an amino acid sequence of SEQ ID NO:1 and a CD3ζ intracellular signaling domain comprising an amino acid sequence of SEQ ID NO:50. In some embodiments, a CAR provided herein comprises an intracellular domain having a 2B4 intracellular signaling domain and a CD3ζ intracellular signaling domain. In specific embodiments, a CAR provided herein comprises an intracellular domain having a 2B4 intracellular signaling domain comprising an amino acid sequence of SEQ ID NO:3 and a CD3ζ intracellular signaling domain comprising an amino acid sequence of SEQ ID NO:50. In some embodiments, a CAR provided herein comprises an intracellular domain having a 2B4 intracellular signaling domain and a FcγRI intracellular signaling domain. In specific embodiments, a CAR provided herein comprises an intracellular domain having a 2B4 intracellular signaling domain comprising an amino acid sequence of SEQ ID NO:3 and a FcγRI intracellular signaling domain comprising an amino acid sequence of SEQ ID NO:53. In some embodiments, a CAR provided herein comprises an intracellular domain having a DECTIN-1 intracellular signaling domain and a CD3ζ intracellular signaling domain. In specific embodiments, a CAR provided herein comprises an intracellular domain having a DECTIN-1 intracellular signaling domain comprising an amino acid sequence of SEQ ID NO:4 and a CD3ζ intracellular signaling domain comprising an amino acid sequence of SEQ ID NO:50. In some embodiments, a CAR provided herein comprises an intracellular domain having a DECTIN-1 intracellular signaling domain and a FcγRI intracellular signaling domain. In specific embodiments, a CAR provided herein comprises an intracellular domain having a DECTIN-1 intracellular signaling domain comprising an amino acid sequence of SEQ ID NO:4 and a FcγRI intracellular signaling domain comprising an amino acid sequence of SEQ ID NO:53. In some embodiments, a CAR provided herein comprises an intracellular domain having a Lox1c intracellular signaling domain and a CD3ζ intracellular signaling domain. In specific embodiments, a CAR provided herein comprises an intracellular domain having a Lox1c intracellular signaling domain comprising an amino acid sequence of SEQ ID NO:2 and a CD3ζ intracellular signaling domain comprising an amino acid sequence of SEQ ID NO:50. In some embodiments, a CAR provided herein comprises an intracellular domain having a Lox1c intracellular signaling domain and a FcγRI intracellular signaling domain. In specific embodiments, a CAR provided herein comprises an intracellular domain having a Lox1c intracellular signaling domain comprising an amino acid sequence of SEQ ID NO:2 and a FcγRI intracellular signaling domain comprising an amino acid sequence of SEQ ID NO:53. In some embodiments, a CAR provided herein comprises an intracellular domain having a CD80 intracellular signaling domain and a CD3ζ intracellular signaling domain. In specific embodiments, a CAR provided herein comprises an intracellular domain having a CD80 intracellular signaling domain comprising an amino acid sequence of SEQ ID NO:42 or SEQ ID NO:43, and a CD3ζ intracellular signaling domain comprising an amino acid sequence of SEQ ID NO:50. In specific embodiments, a CAR provided herein comprises an intracellular domain having a CD80 intracellular signaling domain comprising an amino acid sequence of SEQ ID NO:42, and a CD3ζ intracellular signaling domain comprising an amino acid sequence of SEQ ID NO:50. In specific embodiments, a CAR provided herein comprises an intracellular domain having a CD80 intracellular signaling domain comprising an amino acid sequence of SEQ ID SEQ ID NO:43, and a CD3ζ intracellular signaling domain comprising an amino acid sequence of SEQ ID NO:50. In some embodiments, a CAR provided herein comprises an intracellular domain having a CD80 intracellular signaling domain and a FcγRI intracellular signaling domain. In specific embodiments, a CAR provided herein comprises an intracellular domain having a CD80 intracellular signaling domain comprising an amino acid sequence of SEQ ID NO:42 or SEQ ID NO:43 and a FcγRI intracellular signaling domain comprising an amino acid sequence of SEQ ID NO:53. In specific embodiments, a CAR provided herein comprises an intracellular domain having a CD80 intracellular signaling domain comprising an amino acid sequence of SEQ ID NO:42 and a FcγRI intracellular signaling domain comprising an amino acid sequence of SEQ ID NO:53. In specific embodiments, a CAR provided herein comprises an intracellular domain having a CD80 intracellular signaling domain comprising an amino acid sequence of SEQ ID NO:43 and a FcγRI intracellular signaling domain comprising an amino acid sequence of SEQ ID NO:53. In some embodiments, a CAR provided herein comprises an intracellular domain having a CD40 intracellular signaling domain and a CD3ζ intracellular signaling domain. In specific embodiments, a CAR provided herein comprises an intracellular domain having a CD40 intracellular signaling domain comprising an amino acid sequence of SEQ ID NO:7 and a CD3ζ intracellular signaling domain comprising an amino acid sequence of SEQ ID NO:50. In specific embodiments, a CAR provided herein does not comprise an intracellular domain having a CD86 intracellular signaling domain comprising an amino acid sequence of SEQ ID NO:1 and a FcγRI intracellular signaling domain comprising an amino acid sequence of SEQ ID NO:53.

### 5.4. Polynucleotides

In one aspect, provided herein are polynucleotides that encode a CAR of the present disclosure, such as a CAR described in Section 5.3. In some embodiments, the polynucleotide comprises DNA (e.g., cDNA). In some embodiments, the polynucleotide comprises RNA (e.g., mRNA). In certain embodiments, the polynucleotide is isolated. In certain embodiments, the polynucleotide is substantially pure.

### 5.4.1. Promoter

In certain aspects, provided herein is a polynucleotide that encodes a CAR (such as a polynucleotide described in Section 5.4), wherein the polynucleotide is operatively linked to a promoter. In some embodiments, the promoter is a constitutively active promoter. Constitutively active promoters are known in the art, and any suitable constitutively active promoter capable of expressing the CAR in a mammalian cell, such as the modified mammalian cells described in Section 5.1 can be used. Non-limiting examples of a constitutively active promoter include, for example, the elongation factor-1 alpha (EF1α) promoter, the cytomegalovirus (CMV) promoter, the cytomegalovirus (CMV) enhancer fused to the chicken beta-actin (CAG) promoter, the T7 promoter, and the phosphoglycerate kinase (PGK) promoter. In some embodiments, the polynucleotide provided herein is operably linked to a EF1α promoter. In some embodiments, the polynucleotide provided herein is operably linked to a CAG promoter. In some embodiments, the polynucleotide provided herein is operably linked to a PGK promoter. In some embodiments, the polynucleotide provided herein is operably linked to a CAG promoter. In some embodiments, the polynucleotide provided herein is operably linked to a T7 promoter.

In certain embodiments, the polynucleotide encoding the CAR is operatively linked to an endogenous promoter of an essential gene. As disclosed in Section 5.2, the polynucleotide encoding the CAR can be integrated into the genome of a modified cell using the SLEEK technology. In certain embodiments, the polynucleotide encoding the CAR is inserted within an endogenous coding sequence of an essential gene in the cell's genome. In certain embodiments, the essential gene encodes a gene product that is required for survival and/or proliferation of the cell. In certain embodiments, the knock-in cassette comprises an exogenous coding sequence for the CAR in frame with and downstream (3′) of an exogenous coding sequence or partial coding sequence encoding the gene product of the essential gene, or a functional variant thereof, and wherein the cell expresses the CAR and the gene product encoded by the essential gene that is required for survival and/or proliferation of the cell, or a functional variant thereof. In certain embodiments, the CAR and the gene product encoded by the essential gene are expressed from the endogenous promoter of the essential gene. In certain embodiments, the essential gene is a housekeeping gene. In certain embodiments, the essential gene encodes GAPDH. In certain embodiments, the essential gene is an essential gene as disclosed in International Patent Publication WO2022235811.

Also provided herein is a polynucleotide that encodes a CAR (such as a polynucleotide described in Section 5.4), wherein the polynucleotide is operatively linked to a tissue specific promoter. For example, the tissue specific promoter can be a promoter that selectively enhances or facilitates transcription of a gene in certain cell types (e.g., a monocyte and/or macrophage), but not in other cell types (e.g., a non-monotype or a non-macrophage). A representative non-myeloid reference cell may be a HeLa or a 293T cell.

In some embodiments, the promoter is a myeloid-specific promoter. As used herein, the term “myeloid specific promoter” is intended to mean a promoter that enhances or facilitates transcription of a gene in a myeloid cell (e.g., a macrophage or monocyte), relative to a non-myeloid cell as measured by, for example, a reporter gene assay (e.g., a luciferase, or chemiluminescent reporter assay) or measure of the downstream protein encoded by the gene. In some embodiments, the myeloid specific promoter enhances transcription of a reporter gene (e.g., GFP) in a monocyte or macrophage, relative to a constitutively active promoter (e.g., CMV), and the myeloid specific promoter does not enhance transcription of a reporter gene (e.g., GFP) in a non-myeloid reference cell, relative to a constitutively active promoter (e.g., CMV). A representative non-myeloid reference cell may be, for example, a human intestinal epithelial cell (e.g., Caco-2), a cervix epithelioid carcinoma cell (e.g., HeLa), a human embryonic kidney cell 293 (e.g., HEK-293 or 293T), a T lymphocyte (e.g., Jurkat), or a mouse osteoblast (e.g., Oct-1).

In some embodiments, the myeloid specific promoter enhances transcription in a macrophage and/or monocyte cell approximately 5-fold, relative to in a non-myeloid cell. In some embodiments, the myeloid specific promoter enhances transcription in a macrophage and/or monocyte cell approximately 10-fold, relative to in a non-myeloid cell. In some embodiments, the myeloid specific promoter enhances transcription in a macrophage and/or monocyte cell approximately 20-fold, relative to in a non-myeloid cell. In some embodiments, the myeloid specific promoter enhances transcription in a macrophage and/or monocyte cell approximately 50-fold, relative to in a non-myeloid cell. In some embodiments, the myeloid specific promoter enhances transcription in a macrophage and/or monocyte cell approximately 75-fold, relative to in a non-myeloid cell. In some embodiments, the myeloid specific promoter enhances transcription in a macrophage and/or monocyte cell approximately 100-fold, relative to in a non-myeloid cell. In some embodiments, the myeloid specific promoter enhances transcription in a macrophage and/or monocyte cell about 5-fold to about 100-fold, relative to in a non-myeloid cell. In some embodiments, the myeloid specific promoter enhances transcription in a macrophage and/or monocyte cell about 10-fold to about 100-fold, relative to in a non-myeloid cell. In some embodiments, the myeloid specific promoter enhances transcription in a macrophage and/or monocyte cell about 25-fold to about 100-fold, relative to in a non-myeloid cell. In some embodiments, the myeloid specific promoter enhances transcription in a macrophage and/or monocyte cell about 50-fold to about 100-fold, relative to in a non-myeloid cell.

In some embodiments, the myeloid specific promoter enhances transcription in a macrophage and/or monocyte cell approximately 5-fold, relative to a constitutively active promoter in the macrophage and/or monocyte cell. In some embodiments, the myeloid specific promoter enhances transcription in a macrophage and/or monocyte cell approximately 10-fold, relative to a constitutively active promoter in the macrophage and/or monocyte cell. In some embodiments, the myeloid specific promoter enhances transcription in a macrophage and/or monocyte cell approximately 20-fold, relative to a constitutively active promoter in the macrophage and/or monocyte cell. In some embodiments, the myeloid specific promoter enhances transcription in a macrophage and/or monocyte cell approximately 50-fold, relative to a constitutively active promoter in the macrophage and/or monocyte cell. In some embodiments, the myeloid specific promoter enhances transcription in a macrophage and/or monocyte cell approximately 75-fold, relative to a constitutively active promoter in the macrophage and/or monocyte cell. In some embodiments, the myeloid specific promoter enhances transcription in a macrophage and/or monocyte cell approximately 100-fold, relative to a constitutively active promoter in the macrophage and/or monocyte cell. In some embodiments, the myeloid specific promoter enhances transcription in a macrophage and/or monocyte cell about 5-fold to about 100-fold, relative to a constitutively active promoter in the macrophage and/or monocyte cell. In some embodiments, the myeloid specific promoter enhances transcription in a macrophage and/or monocyte cell about 10-fold to about 100-fold, relative to a constitutively active promoter in the macrophage and/or monocyte cell. In some embodiments, the myeloid specific promoter enhances transcription in a macrophage and/or monocyte cell about 25-fold to about 100-fold, relative to a constitutively active promoter in the macrophage and/or monocyte cell. In some embodiments, the myeloid specific promoter enhances transcription in a macrophage and/or monocyte cell about 50-fold to about 100-fold, relative to a constitutively active promoter in the macrophage and/or monocyte cell.

In some embodiments, the myeloid specific promoter is a native macrophage or a native monocyte promoter, or fragment thereof. For example, the promoter can include the full-length, or a fraction thereof, of the promoter of a gene that is expressed in monocytes and/or macrophages (e.g., CD36, CD68, CD11b, or CSF1R). In some embodiments, the promoter can include the full-length, or a fraction thereof, of the promoter of a gene that is selectively expressed in monocytes and/or macrophages relative to a non-monotype or a non-macrophage, such as, for example, a HeLa or 293T cell.

In some embodiments, the myeloid specific promoter is synthetic promoter. Techniques known to one of skill in the art or described herein (e.g., Section 5.10.5) may be used to generate and screen synthetic promoters. For example, synthetic promoters can be generated by random ligation of myeloid/macrophage cis elements. In some embodiments, synthetic promoter is selected from the group consisting of synthetic promoter-146 (SP146) (GenBank: DQ107383.1), synthetic promoter-107 (SP107) (GenBank: DQ107382.1), and synthetic promoter-60 (SP60) (GenBank: DQ107381.1). In some embodiments, the promoter is a SP146 promoter. In some embodiments, the promoter is a SP107 promoter. In some embodiments, the promoter is a SP60 promoter. Exemplary promoter polynucleotide sequences are provided in Table 5.

In specific embodiments, a promotor provided herein comprises a promoter within any of the nucleotide sequences provided in Table 5. In particular embodiments, a promoter provided herein comprises a promote that is at least 90%, at least 95%, or 100% identical to a polynucleotide sequence provided in Table 5. In specific embodiments, a promoter provided herein comprises a promoter that is at least 90%, at least 95%, or 100% identical to SEQ ID NO:54. In specific embodiments, a promoter provided herein comprises a promoter that is at least 90%, at least 95%, or 100% identical to SEQ ID NO:55. In specific embodiments, a promoter provided herein comprises a promoter that is at least 90%, at least 95%, or 100% identical to SEQ ID NO:56.

TABLE 5
Exemplary Promoter Polynucleotide Sequences

| | | SEQ |
|---|---|---|
| | | ID |
| Promoter | Sequence | NO |
| Synthetic | CTAGCGAGGGCGGACCAGAAAAGGA | 54 |
| promoter- | GAAGTAGGAGCCAAGATTTCCAAAC | |
| 146 (SP146) | TCTGTGGTTGCCTTGCCAAGATTTC | |
| | CAAACTCTGTGGTTGCCTTGCAGAA | |
| | AAGGAGAAGTAGGAGAAGCGACTTC | |
| | CTCTTTCCAGAAGCGACTTCCTCTT | |
| | TCCAGAGGAAGAGGGCGGAGGCTCA | |
| | CAAGGCAACCACAGAGTTTGGAAAT | |
| | CTTGGAAGCGACTTCCTCTTTCCAG | |
| | CAGAAAAGGAGAAGTAGGAGAAGCG | |
| | ACTTCCTCTTTCCAGGTCCGCCCTC | |
| | G | |
| Synthetic | CTAGCGAGGGCGGACAAGCGACTTC | 55 |
| promoter- | CTCTTTCCAGCAGAAAAGGAGAAGT | |
| 107 (SP107) | AGGAGCCAAGATTTCCAAACTCTGT | |
| | GGTTGCCTTGAAGCGACTTCCTCTT | |
| | TCCAGAAGCGACTTCCTCTTTCCAG | |
| | CAGAAAAGGAGAAGTAGGAGCTCCT | |
| | ACTTCTCCTTTTCTGCCAAGATTTC | |
| | CAAACTCTGTGGTTGCCTTGCTCCT | |
| | ACTTCTCCTTTTCTGCAGAAAAGGA | |
| | GAAGTAGGAGCTGGAAAGAGGAAGT | |
| | CGCTTGAGGGCGGACG | |
| Synthetic | CTAGCGTCCGCCCTCAGGAAGAGGG | 56 |
| promoter-60 | CGGAGGCTCACCAAGATTTCCAAAC | |
| (SP60) | TCTGTGGTTGCCTTGCCAAGATTTC | |
| | CAAACTCTGTGGTTGCCTTGCCAAG | |
| | ATTTCCAAACTCTGTGGTTGCCTTG | |
| | TGAGCCTCCGCCCTCTTCCTAAGCG | |
| | ACTTCCTCTTTCCAGGAGGGCGGAC | |
| | G | |

### 5.5. Vectors

In some aspects, provided herein is a vector comprising a polynucleotide encoding a CAR (such as a polynucleotide described in Section 5.4). In some embodiments, the vector is viral vector. In some embodiments, the viral vector is selected from the group consisting of an adenoviral vector, a lentiviral vector, and a retroviral vector. In specific embodiments, the vector is an adenoviral vector. In specific embodiments, the vector is a lentiviral vector. In specific embodiments, the vector is a retroviral vector.

In some embodiments, the vector comprises one or more selection markers. Non-limiting examples of a selection marker include, for example, drug resistance genes for G418 (neo), puromycin (pac), hygromycin B (hph), zeocin (zeo), blasticidin S (bsd), and histidinol (hisD), as well selection markers suitable for fluorescence-activated cell sorting (FACS), such as, green fluorescent protein (GFP), yellow fluorescent protein (YFP), mCherry, and cyan fluorescent protein (CFP).

In specific embodiments, the one or more selection markers are operably linked to a promoter that is different from the promoter that regulates expression of the CAR. In some embodiments, the one or more selection markers are operably linked to a promoter selected from the group consisting of EF1α, CAG and PGK. In some embodiments, the one or more selection markers are operably linked to a EF1α promoter. In some embodiments, the one or more selection markers are operably linked to a CAG promoter. In some embodiments, the one or more selection markers are operably linked to a PGK promoter. In some embodiments, the vector comprises constitutive expression or for inducible expression. In some embodiments, the vector comprises constitutive expression. In some embodiments, the vector comprises inducible expression. The selection of promoters, e.g., strong, weak, tissue-specific, inducible and developmental-specific, is within the ordinary skill of the artisan.

In some embodiments, the vector comprises two or more selection markers that are multicistronic. In some embodiments, one selection marker (e.g., a drug resistance gene) is upstream a 2A peptide (e.g., P2A, T2A, E2A and F2A) and another selection marker (e.g., a selection marker suitable for fluorescence-activated cell sorting (FACS)) is downstream the 2A peptide. In some embodiments, one selection marker (e.g., a selection marker suitable for fluorescence-activated cell sorting (FACS)) is upstream a 2A peptide (e.g., P2A, T2A, E2A and F2A) and another selection marker (e.g., a drug resistance gene) is downstream the 2A peptide. In some embodiments, one selection marker (e.g., a drug resistance gene) is upstream an Internal Ribosome Entry Site (IRES) element and another selection marker (e.g., a selection marker suitable for fluorescence-activated cell sorting (FACS)) is downstream the IRES element. In some embodiments, one selection marker (e.g., a selection marker suitable for fluorescence-activated cell sorting (FACS)) is upstream a IRES element and another selection marker (e.g., a drug resistance gene) is downstream the IRES element.

TABLE 6
2A peptides

| | | SEQ |
|---|---|---|
| Peptide | Amino acid sequence* | ID NO |
| T2A | (GSG)EGRGSLLTCGDVEENPGP | 57 |
| P2A | (GSG)ATNFSLLKQAGDVEENPGP | 58 |
| E2A | (GSG)QCTNYALLKLAGDVESNPGP | 59 |
| F2A | (GSG)VKQTLNFDLLKLAGDVESNPGP | 60 |

*(GSG) residues can be added to the 5′ end of the peptide to improve cleavage efficiency.

In some embodiments, the vector is designed for transient expression, stable expression, or both. In some embodiments, the vector is designed for stable expression. In some embodiments, the vector is designed for transient expression.

### 5.6. Polypeptides

In one aspect, provided herein is a CAR (such as a CAR describe in Section 5.3) comprising an antigen recognition moiety polypeptide (such as an antigen recognition moiety described in Section 5.3.1), a hinge domain polypeptide (such as a hinge domain described in Section 5.3.2), a transmembrane domain polypeptide (such as a transmembrane domain described in Section 5.3.3), and an intracellular domain polypeptide (such as an intracellular domain described in Section 5.3.4).

### 5.7. Compositions

In one aspect, provided herein is a composition comprising a modified cell of the present disclosure (such as a modified cell described in Section 5.1). In some embodiments, provided herein is a composition comprising a modified pluripotent cell of the present disclosure (such as a modified pluripotent cell described in Section 5.1.1). In some embodiments, provided herein is a composition comprising a modified myeloid progenitor cell of the present disclosure (such as a modified myeloid progenitor cell described in Section 5.1.2). In some embodiments, provided herein is a composition comprising a modified monocyte of the present disclosure (such as a modified monocyte described in Section 5.1.3). In some embodiments, provided herein is a composition comprising a modified macrophage of the present disclosure (such as a modified macrophage described in Section 5.1.4). In certain embodiments, the modified macrophage provided herein is an immature macrophage. In certain embodiments, the immature macrophage has not been subjected to any maturation or polarization process. In certain embodiments, provided herein is a composition comprising a modified CD11b⁺CD45⁺ cell of the present disclosure (such as a modified CD11b⁺CD45⁺ cell described in Section 5.1.5). In certain embodiments, the modified CD11b⁺CD45⁺ cell disclosed herein expresses a detectable level of CD14 (e.g., CD11b⁺CD45⁺CD14⁺). In certain embodiments, the modified CD11b⁺CD45⁺ cell disclosed herein does not express a detectable level of CD14 (e.g., CD11b⁺CD45⁺CD14⁻). In certain embodiments, the modified CD11b⁺CD45⁺CD14⁻ cell disclosed herein can mature into cells that express a detectable level of CD14. In some embodiments, the modified cell provided herein (such as a modified cell described in Section 5.1) comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified pluripotent cell provided herein comprises genetic disruption of SIGLEC10, SIRPA, and CISH.

In another aspect, provided herein is a pharmaceutical composition comprising a modified cell of the present disclosure (such as a modified cell described in Section 5.1) and a pharmaceutically acceptable carrier. In some embodiments, provided herein is a pharmaceutical composition comprising a modified pluripotent cell of the present disclosure (such as a modified pluripotent cell described in Section 5.1.1) and a pharmaceutically acceptable carrier. In some embodiments, provided herein is a pharmaceutical composition comprising a modified myeloid progenitor cell of the present disclosure (such as a modified myeloid progenitor cell described in Section 5.1.2) and a pharmaceutically acceptable carrier. In some embodiments, provided herein is a pharmaceutical composition comprising a modified monocyte of the present disclosure (such as a modified monocyte described in Section 5.1.3) and a pharmaceutically acceptable carrier. In some embodiments, provided herein is a pharmaceutical composition comprising a modified macrophage of the present disclosure (such as a modified macrophage described in Section 5.1.4) and a pharmaceutically acceptable carrier. In certain embodiments, the modified macrophage provided herein is an immature macrophage. In certain embodiments, the immature macrophage has not been subjected to any maturation or polarization process. In certain embodiments, provided herein is a pharmaceutical composition comprising a modified CD11b⁺CD45⁺ cell of the present disclosure (such as a modified CD11b⁺CD45⁺ cell described in Section 5.1.5) and a pharmaceutically acceptable carrier. In some embodiments, the modified cell provided herein (such as a modified cell described in Section 5.1) comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified cell provided herein comprises genetic disruption of SIGLEC10, SIRPA, and CISH.

In another aspect, provided herein is a pharmaceutical composition comprising an effective amount of a modified cell of the present disclosure (such as a modified cell described in Section 5.1) and a pharmaceutically acceptable carrier. In some embodiments, provided herein is a pharmaceutical composition comprising an effective amount of a modified pluripotent cell of the present disclosure (such as a modified pluripotent cell described in Section 5.1.1) and a pharmaceutically acceptable carrier. In some embodiments, provided herein is a pharmaceutical composition comprising an effective amount of a modified myeloid progenitor cell of the present disclosure (such as a modified myeloid progenitor cell described in Section 5.1.2) and a pharmaceutically acceptable carrier. In some embodiments, provided herein is a pharmaceutical composition comprising an effective amount of a modified monocyte of the present disclosure (such as a modified monocyte described in Section 5.1.3) and a pharmaceutically acceptable carrier. In some embodiments, provided herein is a pharmaceutical composition comprising an effective amount of a modified macrophage of the present disclosure (such as a modified macrophage described in Section 5.1.4) and a pharmaceutically acceptable carrier. In certain embodiments, the modified macrophage provided herein is an immature macrophage. In certain embodiments, the immature macrophage has not been subjected to any maturation or polarization process. In certain embodiments, provided herein is a pharmaceutical composition comprising an effective amount of a modified CD11b⁺CD45⁺ cell of the present disclosure (such as a modified CD11b⁺CD45⁺ cell described in Section 5.1.5) and a pharmaceutically acceptable carrier. In some embodiments, the modified cell provided herein (such as a modified cell described in Section 5.1) comprises a genetic disruption of: (a) a signal regulatory protein alpha (SIRPA) gene; (b) a cytokine inducible SH2 containing protein (CISH) gene; and/or (c) a sialic acid-binding Ig-like lectin 10 (SIGLEC10) gene. In some embodiments, the modified cell provided herein comprises genetic disruption of SIRPA. In some embodiments, the modified cell provided herein comprises genetic disruption of CISH. In some embodiments, the modified cell provided herein comprises genetic disruption of SIGLEC10. In specific embodiments, the modified cell provided herein comprises genetic disruption of SIRPA and CISH. In certain embodiments, the modified cell provided herein comprises genetic disruption of SIRPA and SIGLEC10. In further embodiments, the modified cell provided herein comprises genetic disruption of SIGLEC10 and CISH. In still further embodiments, the modified cell provided herein comprises genetic disruption of SIGLEC10, SIRPA, and CISH.

As used herein, the term “pharmaceutically acceptable” when used in reference to a carrier, is intended to mean that the carrier, diluent or excipient is not toxic or otherwise undesirable, (i.e., the material may be administered to a subject without causing any undesirable biological effects), and it is compatible with the other ingredients of the formulation. The term “carrier” refers to a diluent, adjuvant, excipient, or vehicle with which the therapeutic is administered. Such pharmaceutical carriers can be sterile liquids, such as saline solutions. A saline solution can be a carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents.

In some embodiments, the pharmaceutical composition comprising a modified cell of the present disclosure (such as a modified cell described in Section 5.1) and a pharmaceutically acceptable carrier further comprises one or more agents. In some embodiments, the pharmaceutical composition comprising a modified monocyte of the present disclosure (such as a modified monocyte described in Section 5.1.3) and a pharmaceutically acceptable carrier further comprises one or more agents. In some embodiments, the pharmaceutical composition comprising a modified macrophage of the present disclosure (such as a modified macrophage described in Section 5.1.4) and a pharmaceutically acceptable carrier further comprises one or more agents. In certain embodiments, the modified macrophage provided herein is an immature macrophage. In certain embodiments, the immature macrophage has not been subjected to any maturation or polarization process. In certain embodiments, the pharmaceutical composition comprising a modified CD11b⁺CD45⁺ cell of the present disclosure (such as a modified CD11b⁺CD45⁺ cell described in Section 5.1.5) and a pharmaceutically acceptable carrier further comprises one or more agents. In some embodiments, the one or more agents comprise a tumor antigen-targeting antibody. In some embodiments, the one or more agents comprise two tumor antigen-targeting antibodies. In some embodiments, the two tumor antigen-targeting antibodies target different tumor antigens. In some embodiments, the agent comprises an anti-CD24 antibody. In some embodiments, the agent comprises an anti-SIGLEC10 antibody. In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody. In some embodiments, the agent comprises a CD20 antibody (e.g., rituximab). In some embodiments, the agent comprises an anti-HER2/neu antibody (e.g., trastuzumab). In some embodiments, the agent comprises an anti-EGFR antibody (e.g., cetuximab). In some embodiments, the agent comprises an anti-TROP2 antibody (e.g., secukinumab). In some embodiments, the one or more agents comprise an anti-EGFR antibody (e.g., cetuximab) and an anti-TROP2 antibody (e.g., secukinumab). In certain embodiments, the modified cell of the present disclosure is precomplexed with one or more agents.

In some embodiments, the pharmaceutical composition comprising a modified cell of the present disclosure (such as a modified cell described in Section 5.1) and a pharmaceutically acceptable carrier further comprises one or more agents. In some embodiments, the pharmaceutical composition comprising an effective amount of a modified monocyte of the present disclosure (such as a modified monocyte described in Section 5.1.3) and a pharmaceutically acceptable carrier further comprises one or more agents. In some embodiments, the pharmaceutical composition comprising an effective amount of a modified macrophage of the present disclosure (such as a modified macrophage described in Section 5.1.4) and a pharmaceutically acceptable carrier further comprises one or more agents. In certain embodiments, the modified macrophage provided herein is an immature macrophage. In certain embodiments, the immature macrophage has not been subjected to any maturation or polarization process. In certain embodiments, the pharmaceutical composition comprising an effective amount of a modified CD11b⁺CD45⁺ cell of the present disclosure (such as a modified CD11b⁺CD45⁺ cell described in Section 5.1.5) and a pharmaceutically acceptable carrier further comprises one or more agents. In some embodiments, the one or more agents comprise a tumor antigen-targeting antibody. In some embodiments, the one or more agents comprise two tumor antigen-targeting antibodies. In some embodiments, the two tumor antigen-targeting antibodies target different tumor antigens. In some embodiments, the agent comprises an anti-CD24 antibody. In some embodiments, the agent comprises an anti-SIGLEC10 antibody. In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody. In some embodiments, the agent comprises a CD20 antibody (e.g., rituximab). In some embodiments, the agent comprises an anti-HER2/neu antibody (e.g., trastuzumab). In some embodiments, the agent comprises an anti-EGFR antibody (e.g., cetuximab). In some embodiments, the agent comprises an anti-TROP2 antibody (e.g., secukinumab). In some embodiments, the one or more agents comprise an anti-EGFR antibody (e.g., cetuximab) and an anti-TROP2 antibody (e.g., secukinumab). In certain embodiments, the modified cell of the present disclosure is precomplexed with one or more agents.

### 5.8. Differentiation of Modified Pluripotent Cells

As provided herein, myeloid progenitor cells can be derived by differentiating a pluripotent cell into a myeloid progenitor cell (such as the modified myeloid progenitor cells described in Section 5.1.2). The myeloid progenitor cell can then be further differentiated into a monocyte (such as the modified monocytes described in Section 5.1.3). The monocyte can be further differentiated into a macrophage (such as the modified macrophages described in Section 5.1.4).

Any differentiation protocol known in the art for differentiating the pluripotent cells (such as the modified pluripotent cells described in Section 5.1.1) into (i) myeloid progenitor cells (such as the modified myeloid progenitor cells described in Section 5.1.2), (ii) monocytes (such as the modified monocytes described in Section 5.1.3), or (iii) macrophages (such as the modified macrophages described in Section 5.1.4 can be used. Non-limiting examples include differentiation protocols described in Cao, et al., Stem cell reports, 12 (6), 1282-1297; Choi, et al., The Journal of clinical investigation 119.9 (2009): 2818-2829; van Wilgenburg, et al., PloS one 8 (8) (2013): e71098; Yanagimachi, et al., PloS One 8 (4) (2013): e59243; WO 2020/078079; Lyadova and Vasiliev, Cell Biosci 12, 96 (2022), and Lyadova et al., Front Cell Dev Biol. 2021 Jun. 2; 9:640703, each of which is incorporated by reference in its entirety.

In some embodiments, the differentiation protocol comprises feeder cells. For example, in certain embodiments, the differentiation protocol can be performed in the presence of mouse bone marrow OP9 stromal cells. In some embodiments, the differentiation protocol involves culturing pluripotent cells (e.g., iPSCs) in the presence of mouse bone marrow OP9 stromal cells until myeloid progenitors are generated. In certain embodiments, the myeloid progenitors are further cultured in the presence of macrophage colony-stimulating factor (M-CSF; also known as CSF1) or granulocyte-macrophage colony-stimulating factor (GM-CSF; CSF2), to induce myeloid specification and macrophage formation.

In some embodiments, the differentiation protocol comprises feeder free cell conditions. In other aspects, the differentiation protocol can be performed in the absence of mouse bone marrow OP9 stromal cells. In OP9-independent protocols, mesoderm and hemogenic endothelium (HE) can be induced through the formation of embryoid bodies (EBs) or EB-independently. Thus, in some embodiments, the differentiation protocol is an OP9-independent protocol and involves EB formation. EBs are 3D cell structures capable of differentiating into all three germ layers, i.e., ectoderm, mesoderm and endoderm. To generate EBs, pluripotent cells (e.g., iPSCs) are cultured in low-adherent conditions which favor cell-cell interactions and EB formation. In certain embodiments, within EBs, mesoderm and HE may form spontaneously, in the absence of any exogenously added factors. In other embodiments, EB formation can be performed in the presence of exogenous mesoderm/HE inducing factors, such as, for example, BMP4, VEGFA and SCF. The subsequent generation of myeloid progenitors and myeloid monocyte-like cells may be driven by culturing EBs in the presence of only two cytokines, IL-3 and M-CSF, or by adding more complex mixes of exogenous factors, which sequentially lead the cells through the hematopoietic and myeloid differentiation stages (e.g., VEGFA, SCF, FGF2, FLT3L, TPO, IL-3, M-CSF) and result to the formation of monocyte-like cells.

In other embodiments, the differentiation protocol is an OP9-independent protocol and does not involve EB-formation. For example, some protocols, such as 2D factor-dependent protocols, induce mesoderm without forming EBs. Exemplary protocols that do not involve EB-formation involve culturing pluripotent cells (e.g., iPSCs) on matrix-coated plates, and all differentiation stages, starting from the stage of mesoderm formation, are induced by multiple exogenous factors (such as BMP4, CHIR99021, Activin A, VEGFA, FGF2, SCF, IL-6, IL-3, and M-CSF). These factors, being added to the cultures sequentially and in different combinations, drive cells through mesoderm/HE->myeloid progenitors->myeloid cell differentiation pathway.

In some aspects, differentiation is performed using a serum-free method. For example, BMP4, VEGF, and SCF can be used to promote mesodermal lineage and prime hemogenic endothelium differentiation during EB formation, and the serum-free X-VIVO 15 (XVIVO) medium (Lonza) can be used during the M-CSF/IL-3-directed myeloid differentiation stage. See, e.g., van Wilgenburg, et al., PloS one 8 (8) (2013): e71098. In other aspects, differentiation is performed using a serum-supplemented medium.

In certain differentiation protocols, monocytes can be differentiated into macrophages using a protocol that comprises M-CSF. In some embodiments, monocytes are contacted with about 40 ng/ml M-CSF. In some embodiments, monocytes are contacted with about 50 ng/ml M-CSF. In some embodiments, monocytes are contacted with about 60 ng/ml M-CSF. In some embodiments, monocytes are contacted with about 70 ng/ml M-CSF. In some embodiments, monocytes are contacted with about 80 ng/ml M-CSF. In some embodiments, monocytes are contacted with about 90 ng/ml M-CSF. In some embodiments, monocytes are contacted with about 100 ng/ml M-CSF. In some embodiments, monocytes are contacted with about 110 ng/ml M-CSF. In some embodiments, monocytes are contacted with about 120 ng/ml M-CSF. In some embodiments, monocytes are contacted with about 130 ng/ml M-CSF. In some embodiments, monocytes are contacted with about 140 ng/ml M-CSF. In some embodiments, monocytes are contacted with about 150 ng/ml M-CSF. In some embodiments, monocytes are contacted with about 40 to about 150 ng/ml M-CSF. In some embodiments, monocytes are contacted with about 50 to about 140 ng/ml M-CSF. In some embodiments, monocytes are contacted with about 60 to about 130 ng/ml M-CSF. In some embodiments, monocytes are contacted with about 70 to about 120 ng/ml M-CSF. In some embodiments, monocytes are contacted with about 80 to about 110 ng/ml M-CSF. In some embodiments, monocytes are contacted with about 50 to about 100 ng/ml M-CSF. In some embodiments, monocytes are contacted with about 100 to about 150 ng/ml M-CSF. In some embodiments, monocytes are contacted with about 75 to about 125 ng/ml M-CSF.

In certain differentiation protocols, monocytes can be differentiated into macrophages using a protocol that comprises GM-CSF. In some embodiments, monocytes are contacted with about 40 ng/ml GM-CSF. In some embodiments, monocytes are contacted with about 50 ng/ml GM-CSF. In some embodiments, monocytes are contacted with about 60 ng/ml GM-CSF. In some embodiments, monocytes are contacted with about 70 ng/ml GM-CSF. In some embodiments, monocytes are contacted with about 80 ng/ml GM-CSF. In some embodiments, monocytes are contacted with about 90 ng/ml GM-CSF. In some embodiments, monocytes are contacted with about 100 ng/ml GM-CSF. In some embodiments, monocytes are contacted with about 110 ng/ml GM-CSF. In some embodiments, monocytes are contacted with about 120 ng/ml GM-CSF. In some embodiments, monocytes are contacted with about 130 ng/ml GM-CSF. In some embodiments, monocytes are contacted with about 140 ng/ml GM-CSF. In some embodiments, monocytes are contacted with about 150 ng/ml GM-CSF. In some embodiments, monocytes are contacted with about 40 to about 150 ng/ml GM-CSF. In some embodiments, monocytes are contacted with about 50 to about 140 ng/ml GM-CSF. In some embodiments, monocytes are contacted with about 60 to about 130 ng/ml GM-CSF. In some embodiments, monocytes are contacted with about 70 to about 120 ng/ml GM-CSF. In some embodiments, monocytes are contacted with about 80 to about 110 ng/ml GM-CSF. In some embodiments, monocytes are contacted with about 50 to about 100 ng/ml GM-CSF. In some embodiments, monocytes are contacted with about 100 to about 150 ng/ml GM-CSF. In some embodiments, monocytes are contacted with about 75 to about 125 ng/ml GM-CSF.

In certain differentiation protocols, monocytes can be differentiated into macrophages using a protocol that comprises GM-CSF and M-CSF. In some embodiments, the differentiation protocol comprises equal parts GM-CSF and M-CSF. For example, in some embodiments, monocytes are contacted with about 40 ng/ml GM-CSF and 40 ng/ml M-CSF. In some embodiments, monocytes are contacted with about 50 ng/ml GM-CSF and 50 ng/ml M-CSF. In some embodiments, monocytes are contacted with about 60 ng/ml GM-CSF and about 60 ng/ml M-CSF. In some embodiments, monocytes are contacted with about 70 ng/ml GM-CSF and about 70 ng/ml M-CSF. In some embodiments, monocytes are contacted with about 80 ng/ml GM-CSF and about 80 ng/ml M-CSF. In some embodiments, monocytes are contacted with about 90 ng/ml GM-CSF and about 90 ng/ml M-CSF. In some embodiments, monocytes are contacted with about 100 ng/ml GM-CSF and about 100 ng/ml M-CSF. In some embodiments, monocytes are contacted with about 110 ng/ml GM-CSF and about 110 ng/ml M-CSF. In some embodiments, monocytes are contacted with about 120 ng/ml GM-CSF and about 120 ng/ml M-CSF. In some embodiments, monocytes are contacted with about 130 ng/ml GM-CSF and about 130 ng/ml M-CSF. In some embodiments, monocytes are contacted with about 140 ng/ml GM-CSF and about 140 ng/ml M-CSF. In some embodiments, monocytes are contacted with about 150 ng/ml GM-CSF and about 150 ng/ml M-CSF. In some embodiments, monocytes are contacted with unequal amounts of GM-CSF and M-CSF. In some embodiments, monocytes are contacted with similar ranges of GM-CSF and M-CSF. In some embodiments, monocytes are contacted with about 40 to about 150 ng/ml GM-CSF, and about 40 to about 150 ng/ml M-CSF. In some embodiments, monocytes are contacted with about 50 to about 140 ng/ml GM-CSF, and about 50 to about 140 ng/ml M-CSF. In some embodiments, monocytes are contacted with about 60 to about 130 ng/ml GM-CSF, and about 60 to about 130 ng/ml M-CSF. In some embodiments, monocytes are contacted with about 70 to about 120 ng/ml GM-CSF, and about 70 to about 120 ng/ml M-CSF. In some embodiments, monocytes are contacted with about 80 to about 110 ng/ml GM-CSF, and about 80 to about 110 ng/ml M-CSF. In some embodiments, monocytes are contacted with about 50 to about 100 ng/ml GM-CSF, and about 50 to about 100 ng/ml M-CSF. In some embodiments, monocytes are contacted with about 100 to about 150 ng/ml GM-CSF, and about 100 to about 150 ng/ml M-CSF. In some embodiments, monocytes are contacted with about 75 to about 125 ng/ml GM-CSF, and about 75 to about 125 ng/ml M-CSF.

In some embodiments, macrophage differentiation protocols optionally further involve contacting monocytes with IL-4 (e.g., about 20-30 ng/ml), IL-10 (e.g., about 20-30 ng/ml), and/or TGF-β (about 20-30 ng/ml) in the presence of M-CSF. In some embodiments, macrophage differentiation protocols optionally further involve contacting monocytes with IL-4 (e.g., about 20-30 ng/ml), IL-10 (e.g., about 20-30 ng/ml), and/or TGF-β (about 20-30 ng/ml) in the presence of GM-CSF. In some embodiments, macrophage differentiation protocols optionally further involve contacting monocytes with IL-4 (e.g., about 20-30 ng/mL), IL-10 (e.g., about 20-30 ng/ml), and/or TGF-β (about 20-30 ng/ml) in the presence of M-CSF and GM-CSF.

In some embodiments, monocytes are contacted with GM-CSF, and/or M-CSF for 6 days to differentiate to macrophages. In some embodiments, monocytes are contacted with M-CSF for 7 days to differentiate to macrophages. In some embodiments, monocytes are contacted with GM-CSF, and/or M-CSF for 8 days to differentiate to macrophages. In some embodiments, monocytes are contacted with GM-CSF, and/or M-CSF for 9 days to differentiate to macrophages. In some embodiments, monocytes are contacted with GM-CSF, and/or M-CSF for 10 days to differentiate to macrophages. In some embodiments, monocytes are contacted with GM-CSF, and/or M-CSF for 11 days to differentiate to macrophages. In some embodiments, monocytes are contacted with GM-CSF, and/or M-CSF for 12 days to differentiate to macrophages. In some embodiments, monocytes are contacted with GM-CSF, and/or M-CSF for 13 days to differentiate to macrophages. In some embodiments, monocytes are contacted with GM-CSF, and/or M-CSF for 14 days to differentiate to macrophages.

In some aspects, the differentiated macrophages can be polarized into an M1 or M2 polarization state. For example, in some embodiments, differentiated macrophages can be polarized into an M1 state by contacting the macrophages with M-CSF (e.g., about 100 ng/ml), LPS (e.g., about 100 ng/ml), and IFNγ (e.g., about 100 ng/ml). In some embodiments, differentiated macrophages can be polarized into an M2 state by contacting the macrophages with M-CSF (e.g., about 100 ng/ml), IL-4 (e.g., about 20 ng/ml), and IL-13 (e.g., about 20 ng/ml).

### 5.9. Kits

In one aspect, the present disclosure provides a kit comprises (a) a modified cell of the present disclosure (such as a modified cell described in Section 5.1) and (b) one or more agents. In one aspect, the present disclosure provides a kit comprises (a) a modified monocyte of the present disclosure (such as a modified monocyte described in Section 5.1.3) and (b) one or more agents. In one aspect, the present disclosure provides a kit comprises (a) a modified macrophage of the present disclosure (such as a modified macrophage described in Section 5.1.4) and (b) one or more agents. In one aspect, the present disclosure provides a kit comprises (a) a modified CD11b⁺CD45⁺ cell of the present disclosure (such as a modified CD11b⁺CD45⁺ cell described in Section 5.1.5) and (b) one or more agents. In one aspect, the present disclosure provides a kit comprises (a) an effective amount of a modified monocyte of the present disclosure (such as a modified monocyte described in Section 5.1.3) and (b) one or more agents. In one aspect, the present disclosure provides a kit comprises (a) an effective amount of a modified macrophage of the present disclosure (such as a modified macrophage described in Section 5.1.4) and (b) one or more agents. In one aspect, the present disclosure provides a kit comprises (a) an effective amount of a modified CD11b⁺CD45⁺ cell of the present disclosure (such as a modified CD11b⁺CD45⁺ cell described in Section 5.1.5) and (b) one or more agents. In some embodiments, the kit further comprises a pharmaceutically acceptable carrier.

In some embodiments, the modified cell (e.g., monocyte, macrophage, or CD11b⁺CD45⁺ cell) and the one or more agents are packaged into suitable packaging material (e.g., containers). In some embodiments, (a) the modified cell (e.g., monocyte, macrophage, or CD11b⁺CD45⁺ cell) and (b) the one or more agents are packaged into the same packaging material (e.g., the same containers). In some embodiments, (a) the modified cell (e.g., monocyte, macrophage, or CD11b⁺CD45⁺ cell) and (b) the one or more agents are packaged into different packaging materials (e.g., different containers). In certain embodiments, (a) the modified cell (e.g., monocyte, macrophage, or CD11b⁺CD45⁺ cell) and (b) the one or more agents packaged into different packaging materials (e.g., different containers) are to be separately administered (e.g., separately administered at different time or through different administering routes).

As used herein, the term “packaging material” refers to a physical structure housing the components of the kit. In certain embodiments, the packaging material can maintain the components sterilely, and can be made of material commonly used for such purposes (e.g., paper, corrugated fiber, glass, plastic, foil, ampoules, vials, tubes, etc.).

In some embodiments, the one or more agents comprise a tumor antigen-targeting antibody. In some embodiments, the one or more agents comprise two tumor antigen-targeting antibodies. In some embodiments, the two tumor antigen-targeting antibodies target different tumor antigens. In some embodiments, the agent comprises an anti-CD24 antibody. In some embodiments, the agent comprises an anti-SIGLEC10 antibody. In some embodiments, the agent comprises an anti-CD47 antibody. Non-limiting examples of suitable anti-CD47 antibodies include clones B6H12, 5F9, 8B6, and C3 (see, e.g., WO 2011/143624, incorporated by reference in its entirety). In some embodiments, the agent comprises a soluble CD47 polypeptide. In some embodiments, the agent comprises an anti-SIRPα antibody. In some embodiments, the agent comprises a CD20 antibody (e.g., rituximab). In some embodiments, the agent comprises an anti-HER2/neu antibody (e.g., trastuzumab). In some embodiments, the agent comprises an anti-EGFR antibody (e.g., cetuximab). In some embodiments, the agent comprises an anti-TROP2 antibody (e.g., secukinumab). In some embodiments, the one or more agents comprise an anti-EGFR antibody (e.g., cetuximab) and an anti-TROP2 antibody (e.g., secukinumab) In certain embodiments, the modified cell of the present disclosure is precomplexed with one or more agents.

### 5.10. Assays

### 5.10.1. Assays for Detecting Genetic Disruption

Any assay known for measuring RNA (e.g., messenger RNA) or protein expression can be used to detect genetic disruption of the genes described herein. Non-limiting examples for measuring RNA expression include PCR based assays (e.g., reverse transcription-polymerase chain reaction (RT-PCR), including real time PCR and quantitative PCR, and Northern blot analysis), and RNA-sequencing (RNA-seq). Other conventional methods for measuring RNA expression can also be employed as suitable.

Non-limiting examples for measuring protein expressing include immunoassays (e.g., immunoblotting/western blotting, enzyme-linked immunosorbent assay (ELISA)), immunofluorescence microscopy, flow cytometry, and mass spectrometry. Other conventional methods for measuring protein expression can also be employed as suitable.

Genetic disruption performed using CRISPR based methods can be assayed using various techniques known in the art. For example, any assay know for measuring insertions and deletions (indels) or changes to the parental sequence, such as next-generation sequencing, can be used to detect genetic disruption of the genes described herein. Similarly, any assay know for measuring off-target effects, such as next-generation sequencing, can be used to detect the specificity of a genetic disruption.

### 5.10.2. Assays for Evaluating Differentiation

Any assay known for evaluating differentiation of pluripotent cells (e.g., iPSCs) into myeloid progenitor cells, monocytes, or macrophages can be used evaluate the modified cells provided herein. For example, differentiation of the pluripotent cells (such as the pluripotent cells described in Section 5.1.1) into myeloid progenitor cells (such as the modified myeloid progenitor cells described in Section 5.1.2), monocytes (such as the modified monocytes described in Section 5.1.3), macrophages (such as the modified macrophages described in Section 5.1.4), or CD11b⁺CD45⁺ cells (such as the modified CD11b⁺CD45⁺ cells described in Section 5.1.5) can be evaluated using various techniques known in the art suitable for detecting cell morphology, cell phenotype, and/or cell function.

Macrophages obtained using various differentiation protocols generally have a typical macrophage morphology (large vacuolated cells with pseudopodia), express a typical macrophage phenotype (CD14⁺CD11b⁺CD45⁺) and execute the main macrophage functions, such as phagocytosis and the secretion of pro- and anti-inflammatory cytokines. In the cell culture environment macrophages are highly adherent cells. Accordingly, in some embodiments macrophages can be characterized by cell morphology (e.g., size is larger than monocytes), and/or adherent.

Myeloid progenitor cells can be characterized using techniques known to one of skill in the art or described herein. For example, in some embodiments myeloid progenitor cells can be characterized by markers such as Lin, CD34⁺, CD38⁺, and CD45RA⁻. See, e.g., Notta F, et al., Science (2016), 351 (6269), and Pellin, D., et al., Nat Commun 10, 2395 (2019), each of which is incorporated by reference in its entirety.

Monocytes can be characterized using techniques known to one of skill in the art or described herein. For example, in some embodiments monocytes can be characterized by markers such as CD14⁺, CD11b⁺, and CD45⁺. in some embodiments monocytes can be characterized by CD11b⁺, and CD45⁺. In certain embodiments, monocytes express detectable levels of CD11b and CD45 (CD11b⁺CD45⁺). In certain embodiments, monocytes express a detectable level of CD14 (e.g., CD11b⁺CD45⁺CD14⁺). In some embodiments, monocytes are characterized based on their expression of CD14 and CD16. In some embodiments, a subset of monocytes are characterized based on CD14^{high} CD16^{neg} expression. In some embodiments, a subset of monocytes are characterized based on CD14^{high} CD16^{pos} expression. In some embodiments, a subset of monocytes are characterized based on CD14^{low} CD16^{high} expression. In some embodiments, monocytes can be characterized as non-adherent cells. In some embodiments, monocytes can be characterized by cell morphology (e.g., size is smaller than macrophages), but larger than T-, B-, NK-cells.

### 5.10.3. Assays for Measuring Cytokine Secretion

Any assay known in the art for measuring cytokine secretion from a cell can be used. Non-limiting examples include immunoassays (e.g., enzyme-linked immunosorbent assay (ELISA), or radioimmunoprecipitation assay (RIP)), and cell-based in vitro bioassays, and may depend on the type of cytokine seeking detection. For example, the cytokine of interest can be immobilized to a microtiter plate, and a test sample (e.g., a cell culture supernatant sample from a cultured macrophage) can be added to the wells of the plate. If the test sample contains a detectable amount of the cytokine, detection can be achieved using a detection antibody (e.g., anti-human/mouse/rabbit IgG, IgM, IgA, or IgE HRP conjugate, anti-human/mouse/rabbit Fc HRP conjugate). One exemplary assay for measuring secretion of a cytokine (e.g., a pro-inflammatory cytokine) involves measuring the cytokine by ELISA after culturing a cell (e.g., a macrophage) for about 24 hours in the presence of a target antigen. Other conventional methods can also be employed as suitable.

### 5.10.4. Assays for Measuring Phagocytosis

Any assay known in the art for measuring phagocytosis can be used. Non-limiting examples include flow cytometry quantification of phagocytosis, and fluorescent microscopy that employs a pH-sensitive dye. The phagocytosis assay could be performed with bacterial particles or a stably transformed cell line. For example, an exemplary assays involves measuring phagocytosis by co-incubation of a macrophage (e.g., a macrophage expressing a CAR) labeled with a fluorescent reporter (e.g., RFP) and a target cell expressing a target antigen that is labeled with a different fluorescent reporter (e.g., GFP) for a suitable period of time (e.g., about four hours), and measuring the amount of double positive cells (RFP+ and GFP+) by flow cytometry. Phagocytosis Index (PI) was calculated as follows:

$$PI = 100 \times = \frac{\% Double Positive Cells}{\% Double Positive Cells + \% GFP only Cells} .$$

Other conventional methods can also be employed as suitable.

### 5.10.5. Assays for Measuring Promoter Activity

Any assay known in the art for measuring promoter activity in a cell can be used. Non-limiting examples include measuring expression of a reporter gene (e.g., luciferase, or any fluorescence protein, such as GFP, RFP, or YFP) from the promoter of the gene of interest, including fluorescence proteins. For example, an in vitro luciferase activity can be used assayed to determine promoter activity in transiently transfected human THP-1 cells. A ubiquitous/constitutively active promoter, such as the cytomegalovirus (CMV) basic promoter, can be used as a promoter control, and PGL3-p47-86 can be used as a basal activity control. Other conventional methods can also be employed as suitable.

Comparison between promoter activity in a myeloid cell (e.g., monocyte or macrophage) and a non-myeloid reference cell can also be performed. A representative non-myeloid reference cell may be, for example, a human intestinal epithelial cell (e.g., Caco-2), a cervix epithelioid carcinoma cell (e.g., HeLa), a human embryonic kidney cell 293 (e.g., HEK-293 or 293T), a T lymphocyte (e.g., Jurkat), or a mouse osteoblast (e.g., Oct-1).

### 5.10.6. Assays for Measuring Target Cell Killing Activities

Any assay known in the art for measuring target cell killing activities of myeloid cells (e.g., monocytes, macrophages, CD11b⁺CD45⁺ cells) can be used. Non-limiting examples include antibody-dependent cellular phagocytosis (ADCP) assay. In certain embodiments, the myeloid cells are incubated with target tumor cells and antibodies that specifically bind to the target tumor cells. After incubation, the cells are collected for flow cytometry assay. Total target cells are gated by the target cell marker and total myeloid cells are gated by a myeloid cell marker (e.g., CD11b). The percentage of phagocytosis may be defined as: Number of target cells phagocytosed by myeloid cell/total number of target cells. Other conventional methods can also be employed as suitable.

### 5.10.7. Cell Migration Assay

Any assay known in the art for measuring the migration ability of myeloid cells (e.g., monocytes, macrophages, CD11b⁺CD45⁺ cells) towards chemokines can be used. Non-limiting examples include TRANSWELL migration assay. In certain embodiments, the migration assay is performed in a TRANSWELL plate with pore inserts. The upper side of one insert is thinly coated for rat tail type I collagen. Myeloid cells are resuspended in a chemotaxis buffer. Cells are added to the upper chamber and migration medium, with or without chemotactic factors: MCP-1, PMA, or C5a is added to the lower chamber. Cells are allowed to migrate through the insert membrane for a period of time. The inserts are then washed with PBS, and nonmigrating cells remaining on the upper surface of the insert are removed with a cotton swab. The migrated cells on the insert are fixed, stained, and mounted on glass slides. Migration is measured visually by counting using a light microscope. A migration index is calculated by dividing the number of cells that migrated in response to the chemokine by the number of cells that migrated randomly with a reference index >1, indicating chemotaxis.

Other conventional methods can also be employed as suitable.

### 6. EMBODIMENTS

This invention provides the following non-limiting embodiments.

A1. A modified pluripotent cell comprising a polynucleotide encoding a chimeric antigen receptor (CAR) and genetic disruption of:
(a) a signal regulatory protein alpha (SIRPA) gene; and/or
(b) a sialic acid-binding Ig-like lectin 10 (SIGLEC10) gene.
   A2. The modified pluripotent cell of embodiment A1, wherein the pluripotent cell comprises genetic disruption of the SIRPA gene.
   A3. The modified pluripotent cell of embodiment A2, wherein the cell is heterozygous for the genetic disruption of the SIRPA gene
   A4. The modified pluripotent cell of embodiment A2, wherein the cell is homozygous for the genetic disruption of the SIRPA gene.
   A5. The modified pluripotent cell of any one of embodiments A1 to A4, wherein the pluripotent cell comprises genetic disruption of the SIGLEC10 gene.
   A6. The modified pluripotent cell of embodiment A5, wherein the cell is heterozygous for the genetic disruption of the SIGLEC10 gene
   A7. The modified pluripotent cell of embodiment A5, wherein the cell is homozygous for the genetic disruption of the SIGLEC10 gene.
   A8. The modified pluripotent cell of any one of embodiments A1 to A7, wherein the pluripotent cell further comprises genetic disruption of a cytokine inducible SH2 containing protein (CISH) gene.
   A9. The modified pluripotent cell of embodiment A8, wherein the cell is heterozygous for the genetic disruption of the CISH gene
   A10. The modified pluripotent cell of embodiment A8, wherein the cell is homozygous for the genetic disruption of the CISH gene.
   A11. The modified pluripotent cell of any one of embodiments A1 to A10, wherein the CAR comprises a non-lymphoid intracellular signaling domain.
   A12. The modified pluripotent cell of embodiment A11, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A13. The modified pluripotent cell of any one of embodiments A1 to A12, wherein the modified pluripotent cell is an induced pluripotent stem cell (iPSC).
   A14. The modified pluripotent cell of embodiment A13, wherein the iPSC has been reprogrammed from a cell selected from the group consisting of a peripheral blood mononuclear cell (PBMC), CD34⁺ cord blood, a macrophage, a monocyte, and a fibroblast.
   A15. A method of generating a modified pluripotent cell, comprising:
(a) obtaining a pluripotent cell expressing a polypeptide encoding a chimeric antigen receptor (CAR); and
(b) genetically disrupting in the pluripotent cell:
   (i) a signal regulatory protein alpha (SIRPA) gene; and/or
   (ii) a sialic acid-binding Ig-like lectin 10 (SIGLEC10) gene.
      A16. A method of generating a modified pluripotent cell, comprising introducing a polypeptide encoding a chimeric antigen receptor (CAR) into a pluripotent cell comprising genetic disruption of:
(a) a signal regulatory protein alpha (SIRPA) gene; and/or
(b) a sialic acid-binding Ig-like lectin 10 (SIGLEC10) gene.
   A17. A method of generating a modified pluripotent cell, comprising:
(a) introducing a polypeptide encoding a chimeric antigen receptor (CAR) into a pluripotent cell; and
(b) genetically disrupting in the pluripotent cell:
   (i) a signal regulatory protein alpha (SIRPA) gene; and/or
   (ii) a sialic acid-binding Ig-like lectin 10 (SIGLEC10) gene.
      A18. A method of generating a modified pluripotent cell, comprising:
(a) genetically disrupting in the pluripotent cell:
   (i) a signal regulatory protein alpha (SIRPA) gene; and/or
   (ii) a sialic acid-binding Ig-like lectin 10 (SIGLEC10) gene in a pluripotent cell; and
(b) introducing a polypeptide encoding a chimeric antigen receptor (CAR) into the pluripotent cell.
   A19. The method of any one of embodiments A15 to A18, wherein the method comprises genetically disrupting the SIRPA gene.
   A20. The method of embodiment A19, wherein the modified pluripotent cell is heterozygous for the genetic disruption of the SIRPA gene
   A21. The method of embodiment A19, wherein the modified pluripotent cell is homozygous for the genetic disruption of the SIRPA gene.
   A22. The method of any one of embodiments A15 to A21, wherein the method comprises genetically disrupting the SIGLEC10 gene.
   A23. The method of embodiment A22, wherein the modified pluripotent cell is heterozygous for the genetic disruption of the SIGLEC10 gene
   A24. The method of embodiment A22, wherein the modified pluripotent cell is homozygous for the genetic disruption of the SIGLEC10 gene.
   A25. The method of any one of embodiments A15 to A24, wherein the method further comprises genetically disrupting a CISH gene.
   A26. The method of embodiment A25, wherein the modified pluripotent cell is heterozygous for the genetic disruption of the CISH gene
   A27. The method of embodiment A25, wherein the modified pluripotent cell is homozygous for the genetic disruption of the CISH gene.
   A28. The method of any one of embodiments A15 to A27, wherein the CAR comprises a non-lymphoid intracellular signaling domain.
   A29. The method of embodiment A28, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A30. The method of any one of embodiments A15 to A29, wherein the pluripotent cell is an induced pluripotent stem cell (iPSC).
   A31. The method of embodiment A30, wherein the iPSC has been reprogrammed from a cell selected from the group consisting of a peripheral blood mononuclear cell (PBMC), CD34⁺ cord blood, a macrophage, a monocyte, and a fibroblast.
   A32. A homogenous population of modified myeloid progenitor cells comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.
   A33. The population of modified myeloid progenitor cells of embodiment A32, wherein the cells comprise genetic disruption of the SIRPA gene.
   A34. The population of modified myeloid progenitor cells of embodiment A33, wherein the cells are heterozygous for the genetic disruption of the SIRPA gene.
   A35. The population of modified myeloid progenitor cells of embodiment A33, wherein the cells are homozygous for the genetic disruption of the SIRPA gene.
   A36. The population of modified myeloid progenitor cells of any one of embodiments A32 to A35, wherein the cells comprise genetic disruption of the SIGLEC10 gene.
   A37. The population of modified myeloid progenitor cells of embodiment A36, wherein the cells are heterozygous for the genetic disruption of the SIGLEC10 gene
   A38. The population of modified myeloid progenitor cells of embodiment A36, wherein the cells are homozygous for the genetic disruption of the SIGLEC10 gene.
   A39. The population of modified myeloid progenitor cells of any one of embodiments A32 to A38, wherein the cells further comprise genetic disruption of a CISH gene.
   A40. The population of modified myeloid progenitor cells of embodiment A39, wherein the cells are heterozygous for the genetic disruption of the CISH gene
   A41. The population of modified myeloid progenitor cells of embodiment A39, wherein the cells are homozygous for the genetic disruption of the CISH gene.
   A42. The population of modified myeloid progenitor cells of any one of embodiments A32 to A41, wherein the cells are isogenic.
   A43. The population of modified myeloid progenitor cells of any one of embodiments A32 to A42, wherein the CAR comprises a non-lymphoid intracellular signaling domain.
   A44. The population of modified myeloid progenitor cells of embodiment A43, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A45. The population of modified myeloid progenitor cells of any one of embodiments A32 to A44, wherein the cells are derived from induced pluripotent stem cells (iPSCs).
   A46. The population of modified myeloid progenitor cells of embodiment A45, wherein the iPSCs have been reprogrammed from a cell selected from the group consisting of a peripheral blood mononuclear cell (PBMC), CD34⁺ cord blood, a macrophage, a monocyte, and a fibroblast.
   A47. A method of generating the homogeneous population of modified myeloid progenitor cells of any one of embodiments A32 to A46, comprising expanding and differentiating the modified pluripotent cell of any one of embodiments A1 to A14 under conditions sufficient for cell differentiation into a population of myeloid progenitor cells.
   A48. The method of embodiment A47, wherein the modified pluripotent cell is an induced pluripotent stem cell (iPSC).
   A49. A homogenous population of modified monocytes comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.
   A50. The population of modified monocytes of embodiment A49, wherein the modified monocytes comprise genetic disruption of the SIRPA gene.
   A51. The population of modified monocytes of embodiment A50, wherein the modified monocytes are heterozygous for the genetic disruption of the SIRPA gene.
   A52. The population of modified monocytes of embodiment A50, wherein the modified monocytes are homozygous for the genetic disruption of the SIRPA gene.
   A53. The population of modified monocytes of any one of embodiments A49 to A52, wherein the modified monocytes comprise genetic disruption of the SIGLEC10 gene.
   A54. The population of modified monocytes of embodiment A53, wherein the modified monocytes are heterozygous for the genetic disruption of the SIGLEC10 gene
   A55. The population of modified monocytes of embodiment A53, wherein the modified monocytes are homozygous for the genetic disruption of the SIGLEC10 gene.
   A56. The population of modified monocytes of any one of embodiments A49 to A55, wherein the modified monocytes further comprise genetic disruption of a CISH gene.
   A57. The population of modified monocytes of embodiment A56, wherein the modified monocytes are homozygous for the genetic disruption of the CISH gene.
   A58. The population of modified monocytes of embodiment A56, wherein the modified monocytes are homozygous for the genetic disruption of the CISH gene.
   A59. The population of modified monocytes of any one of embodiments A49 to A58, wherein the modified monocytes are isogenic.
   A60. The population of modified monocytes of any one of embodiments A49 to A59, wherein the CAR comprises a non-lymphoid intracellular signaling domain.
   A61. The population of modified macrophages of embodiment A60, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A62. The population of modified monocytes of any one of embodiments A49 to A61, wherein the modified monocytes are derived from induced pluripotent stem cells (iPSCs).
   A63. The population of modified monocytes of embodiment A62, wherein the iPSCs have been reprogrammed from a cell selected from the group consisting of a peripheral blood mononuclear cell (PBMC), CD34⁺ cord blood, a macrophage, a monocyte, and a fibroblast.
   A64. A method of generating the homogeneous population of monocytes of any one of embodiments A49 to A63, comprising expanding and differentiating the modified pluripotent cell of any one of embodiments A1 to A14 under conditions sufficient for cell differentiation into a population of monocytes.
   A65. The method of embodiment A64, wherein the modified pluripotent cell is an induced pluripotent stem cell (iPSC).
   A66. A homogenous population of modified macrophages comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.
   A67. The population of modified macrophages of embodiment A66, wherein the modified macrophages comprise genetic disruption of the SIRPA gene.
   A68. The population of modified macrophages of embodiment A67, wherein the modified macrophages are heterozygous for the genetic disruption of the SIRPA gene.
   A69. The population of modified macrophages of embodiment A67, wherein the modified macrophages are homozygous for the genetic disruption of the SIRPA gene.
   A70. The population of modified macrophages of any one of embodiments A66 to A69, wherein the modified macrophages comprise genetic disruption of the SIGLEC10 gene.
   A71. The population of modified macrophages of embodiment A70, wherein the modified macrophages are heterozygous for the genetic disruption of the SIGLEC10 gene
   A72. The population of modified macrophages of embodiment A70, wherein the modified macrophages are homozygous for the genetic disruption of the SIGLEC10 gene.
   A73. The population of modified macrophages of any one of embodiments A66 to A72, the modified macrophages further comprise genetic disruption of a CISH gene.
   A74. The population of modified macrophages of embodiment A73, wherein the modified macrophages are heterozygous for the genetic disruption of the CISH gene
   A75. The population of modified macrophages of embodiment A73, wherein the modified macrophages are homozygous for the genetic disruption of the CISH gene.
   A76. The population of modified macrophages of any one of embodiments A66 to A75, wherein the modified macrophages are isogenic.
   A77. The population of modified macrophages of any one of embodiments A66 to A76, wherein the CAR comprises a non-lymphoid intracellular signaling domain.
   A78. The population of modified macrophages of embodiment A77, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A79. The population of modified macrophages of any one of embodiments A66 to A78, wherein the modified macrophages are derived from induced pluripotent stem cells (iPSCs).
   A80. The population of modified macrophages of embodiment A79, wherein the iPSCs have been reprogrammed from a cell selected from the group consisting of a peripheral blood mononuclear cell (PBMC), CD34⁺ cord blood, a macrophage, a monocyte, and a fibroblast.
   A81. A method of generating the homogeneous population of macrophages of any one of embodiments A66 to A80 comprising expanding and differentiating the modified pluripotent cell of any one of embodiments A1 to A14 under conditions sufficient for cell differentiation into a population of macrophages.
   A82. The method of embodiment A81, wherein the modified pluripotent cell is an induced pluripotent stem cell (iPSC).
   A83. A homogenous population of modified CD11b⁺CD45⁺ cells comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.
   A84. The population of modified CD11b⁺CD45⁺ cells of A83, wherein the cells comprise genetic disruption of the SIRPA gene.
   A85. The population of modified CD11b⁺CD45⁺ cells of A84, wherein the cells are heterozygous for the genetic disruption of the SIRPA gene.
   A86. The population of modified CD11b⁺CD45⁺ cells of A84, wherein the cells are homozygous for the genetic disruption of the SIRPA gene.
   A87. The population of modified CD11b⁺CD45⁺ cells of any one of A83 to A86, wherein the cells comprise genetic disruption of the SIGLEC10 gene.
   A88. The population of modified CD11b⁺CD45⁺ cells of A87, wherein the cells are heterozygous for the genetic disruption of the SIGLEC10 gene.
   A89. The population of modified CD11b⁺CD45⁺ cells of A87, wherein the cells are homozygous for the genetic disruption of the SIGLEC10 gene.
   A90. The population of modified CD11b⁺CD45⁺ cells of any one of A83 to A89, wherein the cells further comprise genetic disruption of a CISH gene.
   A91. The population of modified CD11b⁺CD45⁺ cells of A90, wherein the cells are heterozygous for the genetic disruption of the CISH gene.
   A92. The population of modified CD11b⁺CD45⁺ cells of A90, wherein the cells are homozygous for the genetic disruption of the CISH gene.
   A93. The population of modified CD11b⁺CD45⁺ cells of any one of A83 to A92, wherein the cells are isogenic.
   A94. The population of modified CD11b⁺CD45⁺ cells of any one of A83 to A93, wherein the CAR comprises a non-lymphoid intracellular signaling domain.
   A95. The population of modified CD11b⁺CD45⁺ cells of A94, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.
   A96. The population of modified CD11b⁺CD45⁺ cells of any one of A83 to A95, wherein the cells are derived from induced pluripotent stem cells (iPSCs).
   A97. The population of modified CD11b⁺CD45⁺ cells of A96, wherein the iPSCs have been reprogrammed from a cell selected from the group consisting of a peripheral blood mononuclear cell (PBMC), CD34⁺ cord blood, a macrophage, a monocyte, and a fibroblast.
   A98. The population of modified CD11b⁺CD45⁺ cells of any one of A83 to A97, wherein the modified CD11b⁺CD45⁺ cell is a CD11b⁺CD45⁺CD14⁻ cell or a CD11b⁺CD45⁺CD14⁺ cell.
   A99. A method of generating the homogeneous population of modified CD11b⁺CD45⁺ cells of any one of A83 to A98, comprising expanding and differentiating the modified pluripotent cell of any one of A1 to A14 under conditions sufficient for cell differentiation into a population of CD11b⁺CD45⁺ cells.
   A100. The method of A99, wherein the modified pluripotent cell is an induced pluripotent stem cell (iPSC).

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations can be within the scope of the following claims.

### 7. EXAMPLES

The examples in this section are offered by way of illustration, and not by way of limitation. The following examples are presented as exemplary embodiments of the invention. They should not be construed as limiting the broad scope of the invention.

### 7.1.1. Example 1: Genetic Disruption of iPSCs

This example demonstrated that iPSCs could be genetically disrupted using CRISPR/MAD7, and successfully differentiated into a monocyte.

Briefly, iPSCs were contacted with MAD7 and sgRNA targeting exon 2 of SIRPA (5′-CGACCUCUCUGAUCCCUGUG-3′ (SEQ ID NO:63)) that targets the genetic sequence 5′-TGACCTCCCTGATCCCTGTG-3′ (SEQ ID NO:64). Homozygous clones were identified by sequencing. Exemplary clones indicated an insertion (C>CT) within the target exon (Table 7).

TABLE 7
SIRPα KO Clone Sequencing Data
VARIANT SUMMARY

| | | TC1133 | | SIRPα KO Clone 1 | | SIRPα KO Clone 2 | |
|---|---|---|---|---|---|---|---|
| Target | Variant | Reads | % | Reads | % | Reads | % |
| SIRPα | Del | 0 | 0.00% | 0 | 0.00% | 0 | 0.00% |
| Exon 2 | | | | | | | |
| SIRPα | Ins | 0 | 0.00% | 59233 | 98.24% | 102329 | 97.49% |
| Exon 2 | | | | | | | |
| SIRPα | Coverage | 70412 | NA | 60297 | NA | 104968 | NA |
| Exon 2 | | | | | | | |

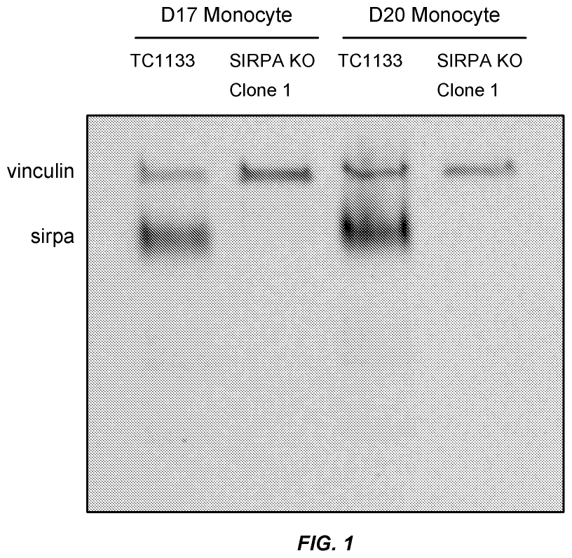

SIRPA KO iPSCs were subsequently differentiated to monocytes. WT (TC1133) and SIRPA KO monocytes were harvested at Day 17 (left of FIG. 1) and Day 20 (right of FIG. 1) of the differentiation protocol, and lysed for protein. Western blotting confirmed the absence of SIRPA expression in SIRPA KO clone 1 on Day 17 and Day 20 (FIG. 1). Vinculin served as the housekeeping gene, and TC1133 cells served as wild-type control cells that expressed SIRPA.

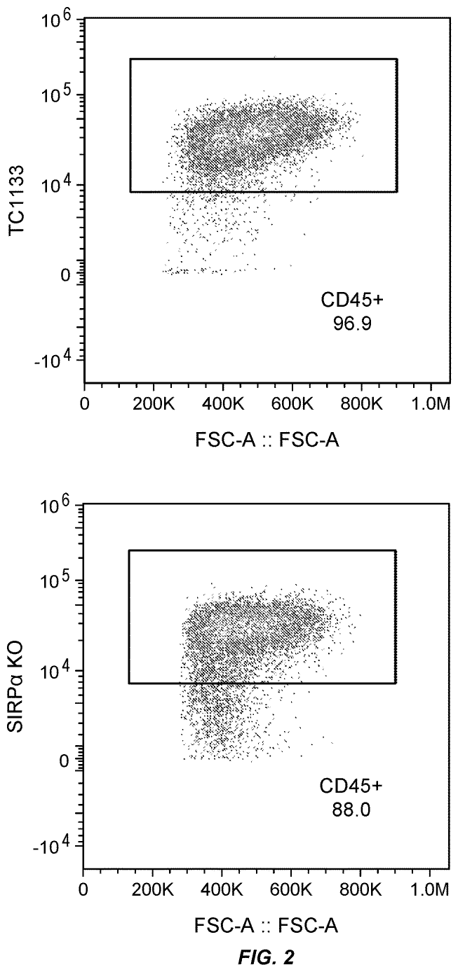

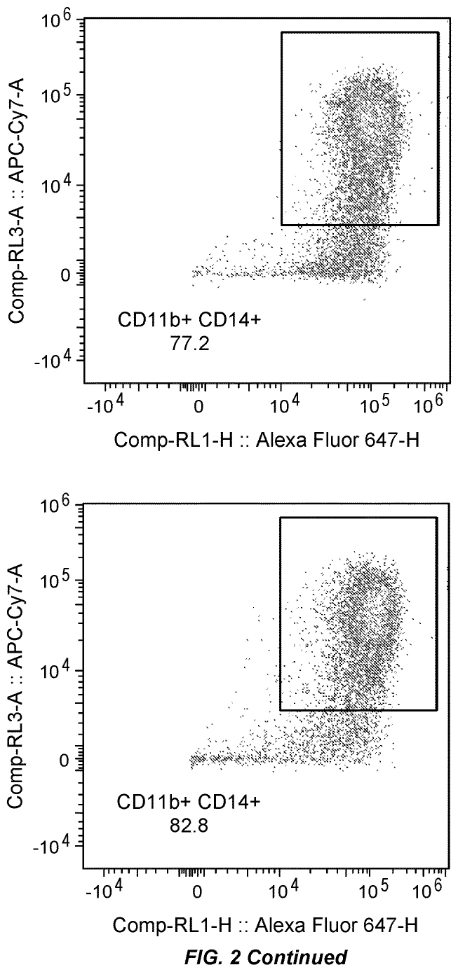

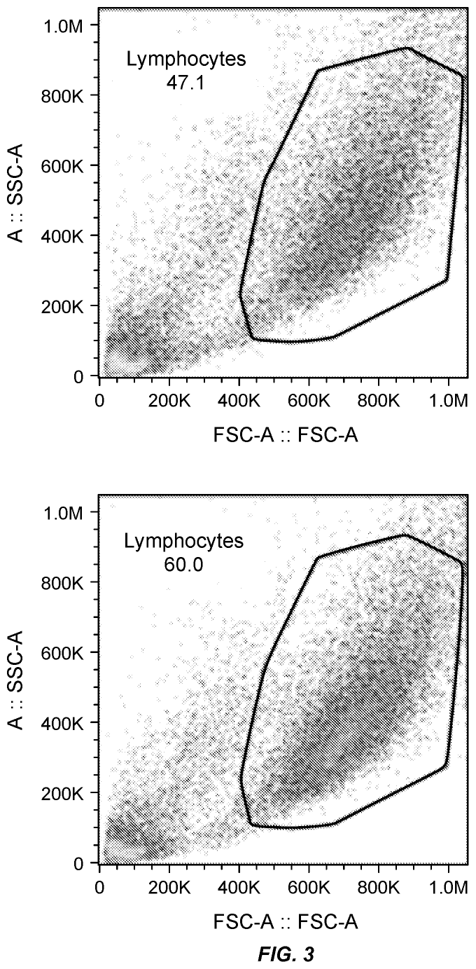

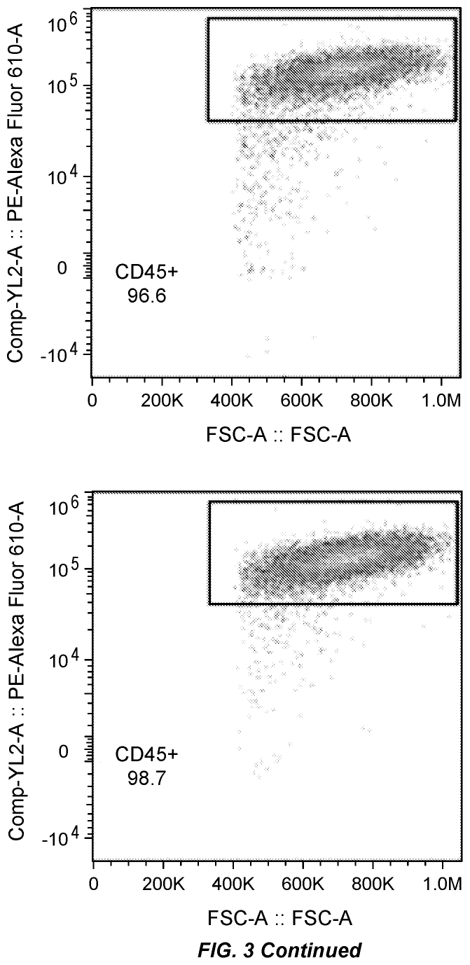

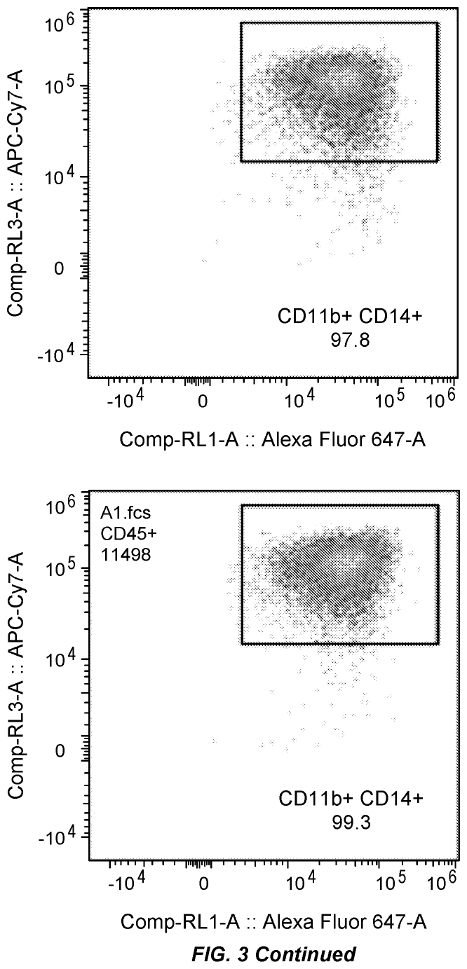

In addition, cells were analyzed by flow cytometry on Day 17 and Day 20 to confirm monocyte purity. Briefly, live singlets were gated on CD45⁺, and then CD11b⁺CD14⁺. CD11b⁺CD14⁺ confirms monocyte purity. As shown in FIG. 2 and Table 8, SIRPA KO monocytes exhibited similar purity levels as wild-type monocytes. Similar results were observed on Day 20 (FIG. 3 and Table 9).

TABLE 8
Day 17 (D 17) Monocytes

| | CD45⁺ (%) | CD11b⁺ CD14⁺ |
|---|---|---|
| TC1133 monocytes | 96.9 | 77.2 |
| SIRPA KO monocytes | 88.0 | 82.8 |

TABLE 9
Day 20 (D 20) Monocytes

| | CD45⁺ (%) | CD11b⁺ CD14⁺ |
|---|---|---|
| TC1133 monocytes | 96.6 | 97.8 |
| SIRPA KO monocytes | 98.7 | 99.3 |

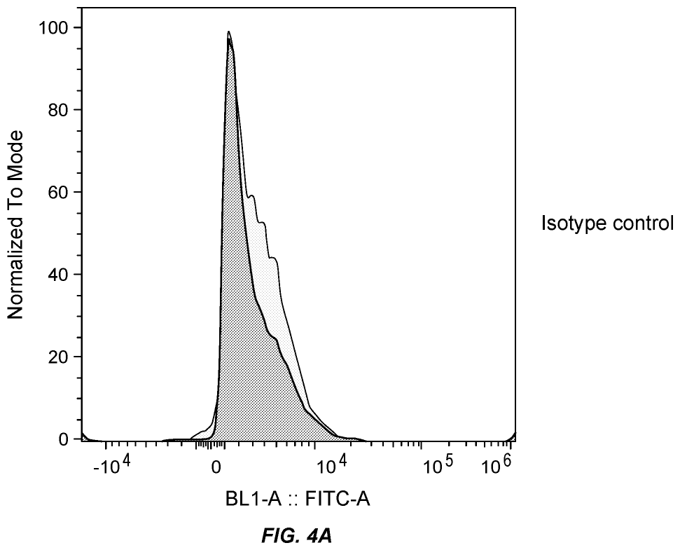

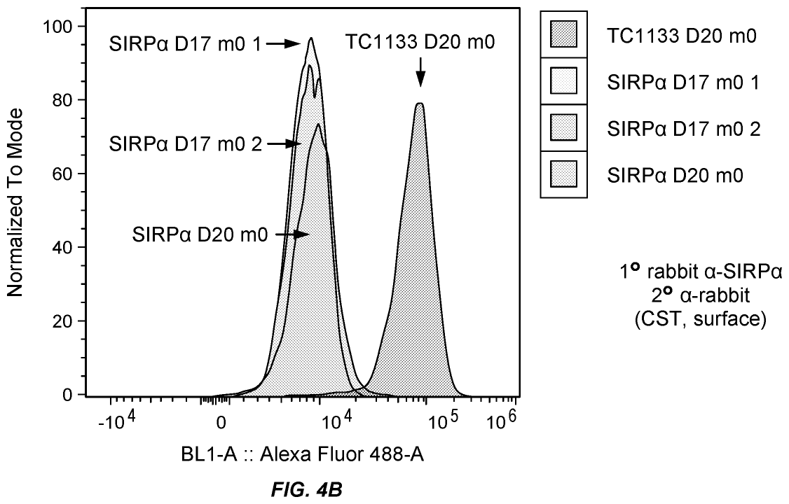

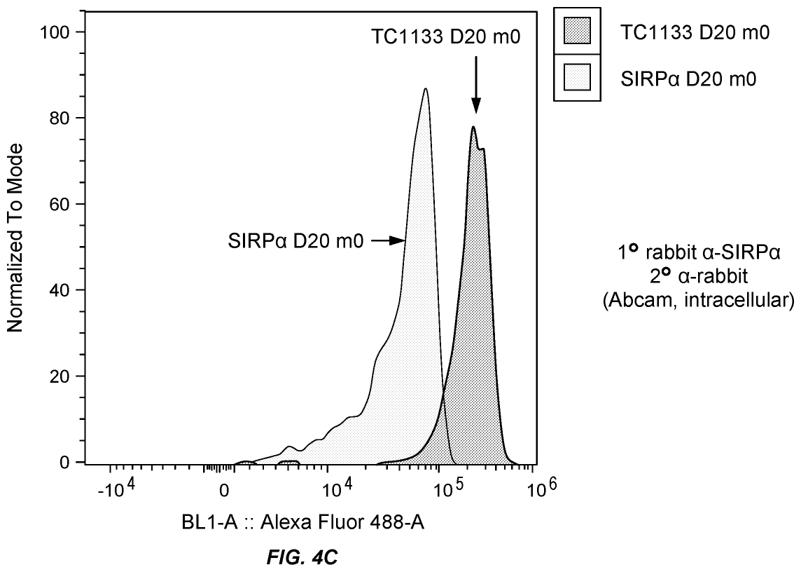

The D17 and D20 iPSC-derived monocytes were further differentiated to iPSC-derived macrophages by treating the monocytes with 100 ng/ml macrophage colony-stimulating factor (M-CSF; also known as colony stimulating factor 1 “CSF1”) for 10 days. Flow cytometry confirmed loss of SIRPα expression in SIRPA KO iPSC-derived macrophages using an antibody that detected cell surface expression of SIRPα (FIG. 4B; Cell Signaling Technologies, CST) or intracellular expression (FIG. 4C; Abcam). Isotype controls were used as a negative control to determine background caused by nonspecific antibody binding (FIG. 4A).

Taken together, this example demonstrated that iPSCs could be genetically modified using CRISPR/MAD7, and successfully differentiated into a monocyte or a macrophage.

### 7.1.2. Example 2: SIRPA KO Monocytes had Increased Killing

This example demonstrated that SIRPA KO monocytes had increased killing relative to monocytes expressing SIRPA.

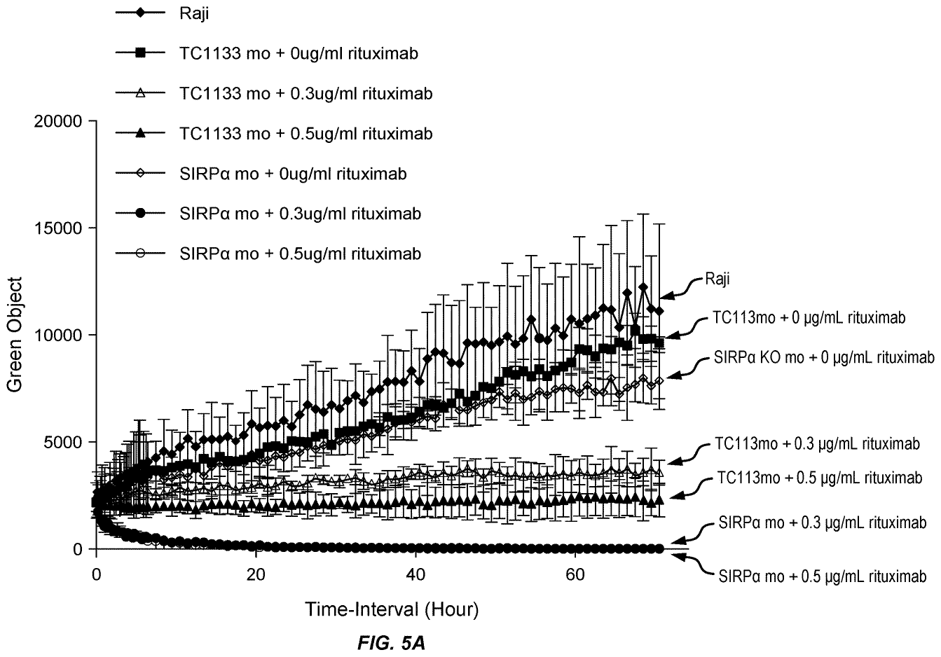

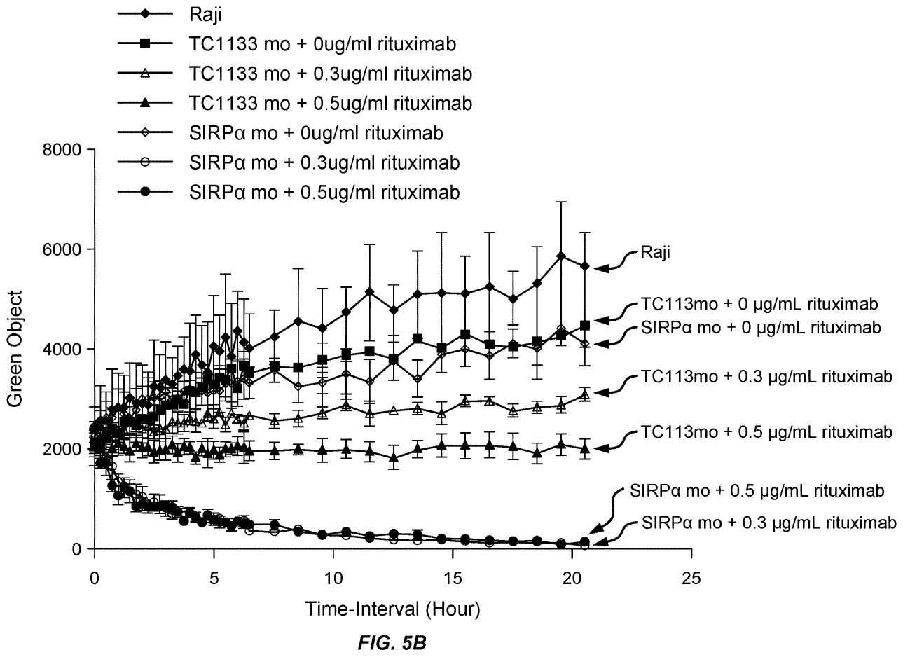

Briefly, SIRPA KO monocyte (“SIRPα mo” or “SIRPα KO mo”) derived from SIRPA KO iPSCs or wild-type control monocytes derived from the TC-113 iPSC-cell line (“TC1133 mo”) were combined with Raji target cells at a 10:1 effector to target ratio. Anti-CD20 (rituximab) antibody was added (0.3 μg/mL or 0.5 μg/mL) and the amount of target cell survival was measured over time (FIG. 5A and FIG. 5B). The results demonstrated that SIRPA KO monocytes have increased killing of Raji cells in comparison to wild-type control monocytes with the addition of 0.3 μg/mL or 0.5 μg/mL rituximab.

In the absence of rituximab, the results demonstrated that SIRPA KO monocytes exhibited a similar amount of target cell killing as the wild-type control monocytes (FIG. 5A and FIG. 5B). Further, both the SIRPA KO monocytes and wild-type TC1133 monocytes exhibited minimal inhibition of the target cell growth in the absence of rituximab, which indicated that the cells exhibited little non-specific killing of the target cells (FIG. 5A and FIG. 5B).

Taken together, this example demonstrated that SIRPA KO monocytes derived from SIRPA KO iPSCs had increased killing relative to monocytes expressing SIRPA derived from wild-type iPSCs.

### 7.1.3. Example 3: SIRPA KO Macrophages had Increased Killing

This example demonstrated that SIRPA KO macrophages had increased killing relative to monocytes expressing SIRPA.

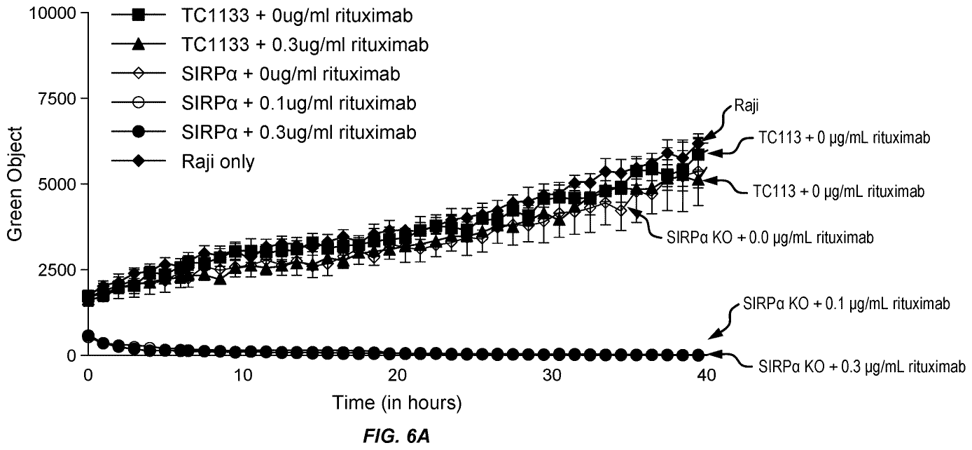

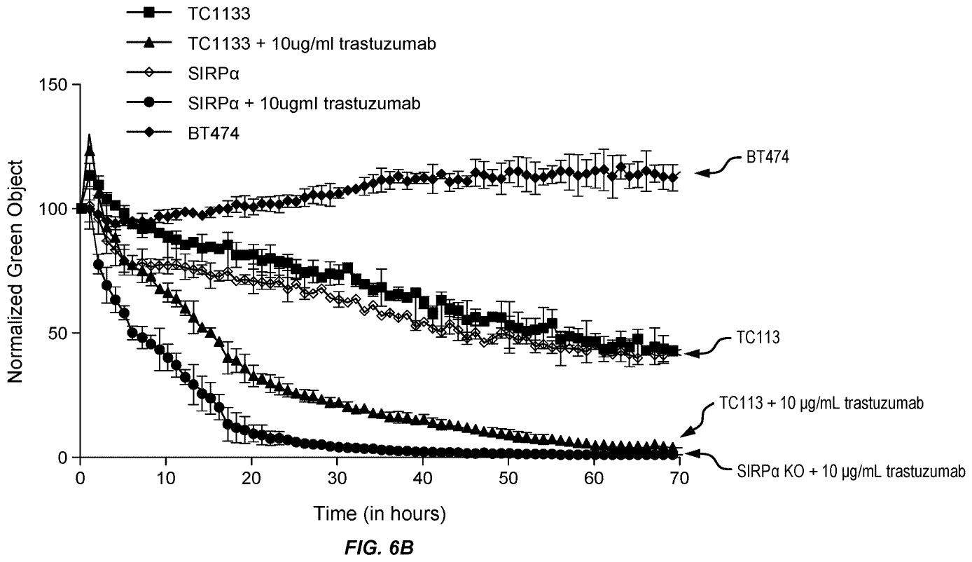

Briefly, SIRPA KO macrophages (“SIRPα” or “SIRPα KO”) derived from SIRPA KO iPSCs or wild-type control macrophages derived from the TC-113 iPSC-cell line (“TC1133”) were combined with Raji target cells at a 3:1 effector cell to target cell ratio. Anti-CD20 (rituximab) antibody was added (0.1 μg/mL or 0.3 μg/mL) and the amount of target cell survival was measured over time (FIG. 6A). The results demonstrated that SIRPA KO macrophages derived from SIRPA KO iPSCs have increased killing of Raji cells in comparison to wild-type control macrophages derived from the TC-113 iPSC-cell line upon the addition of 0.1 μg/mL or 0.3 μg/mL rituximab.

In the absence of rituximab, the results demonstrated that SIRPA KO macrophages derived from SIRPA KO iPSCs exhibited a similar amount of target cell killing in the Raji cell model as the wild-type control macrophages (FIG. 6A). Further, both the SIRPA KO macrophages and wild-type TC1133 macrophages exhibited minimal inhibition of the target cell growth in the absence of rituximab in the Raji cell model, which indicated that the cells exhibited little non-specific killing of the target cells (FIG. 6A).

Similar results were obtained in a solid tumor model using BT474 breast cancer cells. Briefly, 100,000 SIRPA KO macrophages (“SIRPα” or “SIRPα KO”) derived from SIRPA KO iPSCs or 100,000 wild-type control macrophages derived from the TC-113 iPSC-cell line (“TC1133”) were combined with 20,000 BT474 target cells at a 5:1 effector cell to target cell ratio. Anti-HER2/neu (trastuzumab) antibody was added (10 μg/mL) and the amount of target cell survival was measured over time (FIG. 6B). The results demonstrated that SIRPA KO macrophages derived from SIRPA KO iPSCs have increased killing of BT474 cells in comparison to wild-type control macrophages derived from the TC-113 iPSC-cell line upon the addition of 10 μg/mL trastuzumab.

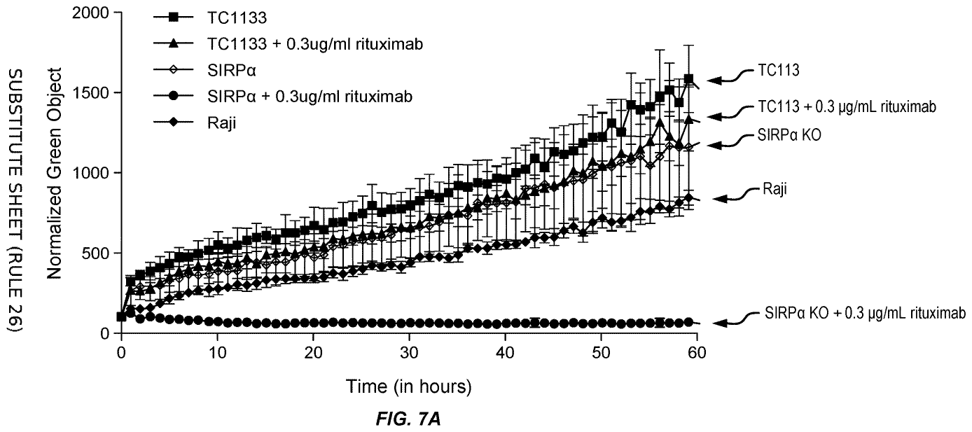

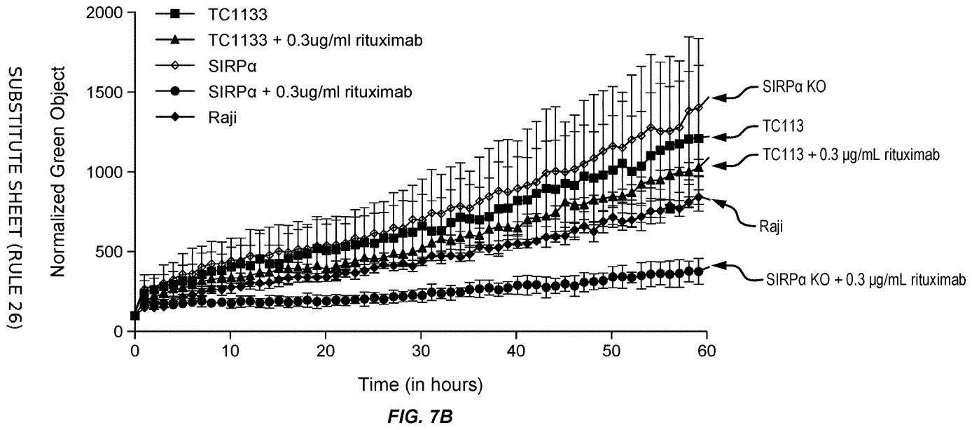

To determine whether SIRPA KO macrophages derived from SIRPA KO iPSCs could exhibit similar efficacy results at lower effector to target ratios, SIRPA KO macrophages derived from SIRPA KO iPSCs or wild-type control macrophages derived from the TC-113 iPSC-cell line (“TC1133”) were combined with Raji target cells at a 3:1 effector cell to target cell ratio (FIG. 7A) or a 1:1 effector to target ratio (FIG. 7B). The results demonstrated that that SIRPA KO macrophages derived from SIRPA KO iPSCs had increased killing of Raji cells in comparison to wild-type control macrophages derived from the TC-113 iPSC-cell line upon the addition of 0.3 μg/mL rituximab at both 3:1 and 1:1 effector to target ratios, and showed little non-specific killing of the target cells.

Taken together, this example demonstrated that SIRPA KO macrophages derived from SIRPA KO iPSCs had increased killing relative to macrophages expressing SIRPA derived from wild-type iPSCs. Further, this example demonstrated that SIRPA KO macrophages exhibited efficient target cell killing at lower effector to target ratio.

### 7.1.4. Example 4: IMACs Pre-Complexed with Rituximab Killed Raji Cells In Vivo

This example demonstrated that macrophages derived from iPSCs (iMACs) precomplexed with a therapeutic antibody killed target cells in vivo.

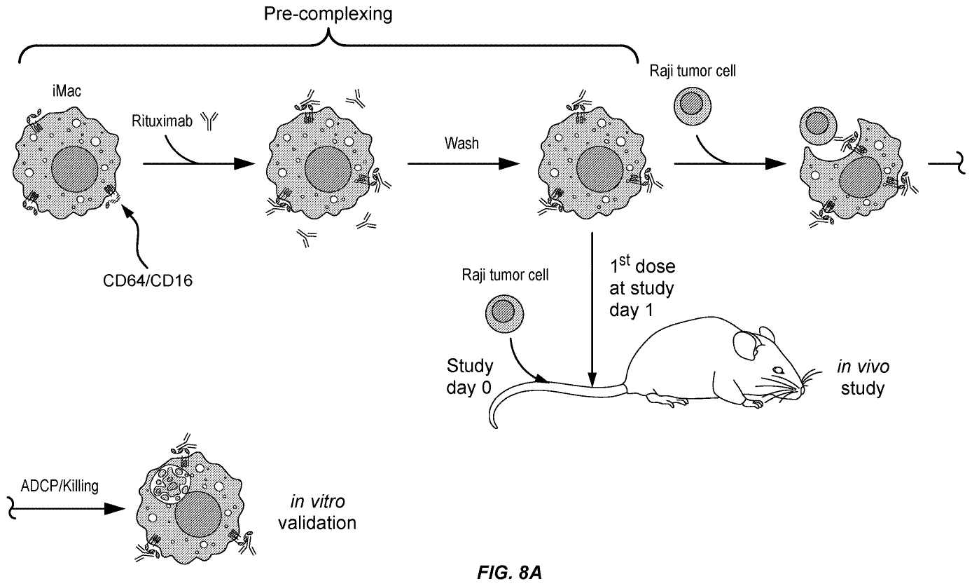

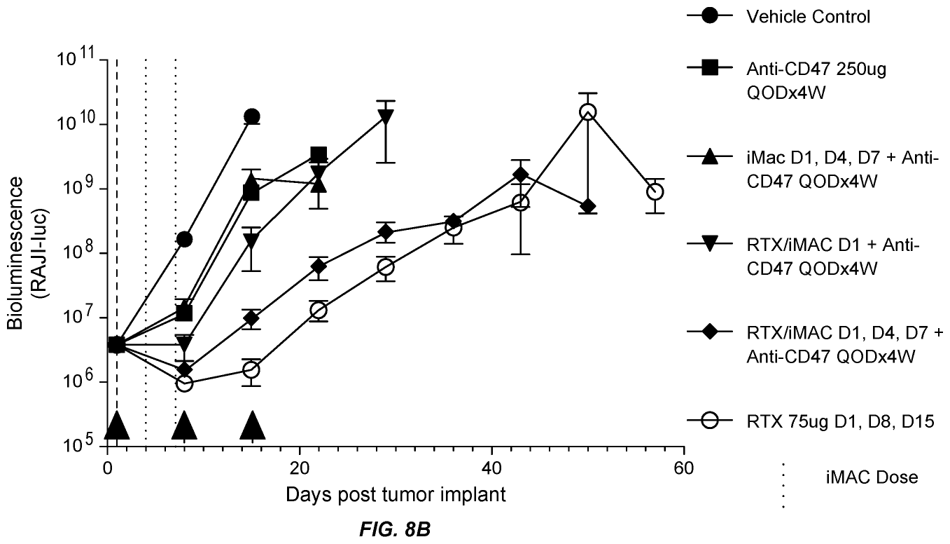

To determine whether macrophages derived from iPSCs were able to synergize with targeting the CD47/SIRPA signaling pathway, six experimental groups of mice were evaluated using the protocols describe in Table 10. Briefly, in one experimental group iMAC cells were administered on days 1 and 4, along with treatment of anti-CD47 to disrupt the SIRPA/CD47 signaling pathway (Group 3). Two other experimental groups (Groups 4 and 5) involved iMAC cells precomplexed with rituximab prior to administration, followed by anti-CD47 administration. A schema of pre-complexing is provided in FIG. 8A. Vehicle control (Group 1), anti-CD47 alone (Group 2), and rituximab (RTX) alone (Group 6) were used as controls. The rituximab alone group was administered at a higher dose (75 μg/dose) relative to the predicted dose of rituximab in the precomplexed iMAC cells (~5 μg), and administration occurred for a longer period of time (administered on days 1, 8, and 15) relative to the precomplexed iMAC cells (day 1 only or days 1, 4, and 7).

TABLE 10
In vivo Study Protocols for iMACs

| Treatment Group | Protocol |
|---|---|
| Group 1: Control (n = 5) | Vehicle Control (Days 1, 4, 7) |
| Group 2: Anti-CD47 (n = 5) | Anti-CD47: QOD × 4 weeks; 250 μg/dose (IP) |
| Group 3: iMAC + Anti-CD47 | iMAC: 1 × 10⁷ cells/mouse administered (IV) on days |
| (n = 5) | 1 and 4; 2.3 × 10⁶ cells/mouse administered (IV) on |
| | day 7; |
| | Anti-CD47: QOD × 4 weeks; 250 μg/dose (IP) |
| Group 4: RTX/iMAC + Anti- | RTX/iMAC: 1 × 10⁷ cells/mouse precomplexed with |
| CD47 (n = 5) | RTX*, administered together (IV) as single dose on |
| | day 1; |
| | Anti-CD47: QOD × 4 weeks; 250 μg/dose (IP) |
| Group 5: RTX/iMAC + Anti- | RTX/iMAC: 1 × 10⁷ cells/mouse precomplexed with |
| CD47 (n = 5) | RTX*, administered together (IV) on days 1 and 4; |
| | 2.3 × 10⁶ cells/mouse (IV), administered on day 7 |
| | Anti-CD47: QOD × 4 weeks; 250 μg/dose (IP) |
| Group 6: RTX (n = 5) | RTX: 75 μg/dose (IP), administered on days 1, 8, and |
| | 15 |

RTX = rituximab;
QOD = every other day;
*predicted RTX injected in precomplexed samples = 5 μg

The results demonstrated that iMACs pre-complexed with rituximab killed Raji cells in vivo. Mice treated with iMACs pre-complexed with rituximab for three days showed improved effector activity (Group 5), relative to Mice treated with iMACs pre-complexed with rituximab for one day (Group 4) (FIG. 8B, diamond and upside-down triangle, respectively). Similarly, both groups of mice treated with iMACs pre-complexed with rituximab exhibited improved effector activity relative to mice treated with anti-CD47 alone or mice treated with iMAC and anti-CD47, but not precomplexed with rituximab (Group 3) (FIG. 8B, square and triangle, respectively). Moreover, the mice treated with iMACs pre-complexed with rituximab for three days showed similar effector activity as mice treated with prolonged high dose rituximab (Group 6), even though the rituximab dose was estimated to be approximately 15 fold lower than the rituximab alone group and treatment occurred for a shorter duration (FIG. 8B, open circle).

Taken together, these results demonstrated that macrophages derived from iPSCs were able to synergize with targeting the CD47/SIRPA signaling pathway to kill Raji cells in vivo.

### 7.1.5. Example 5: SIRPA KO CAR-Macrophages had Increased Killing of Head and Neck Cancer Cells

This example demonstrated that SIRPA KO macrophages had increased killing of head and neck cancer cells relative to macrophages expressing SIRPA.

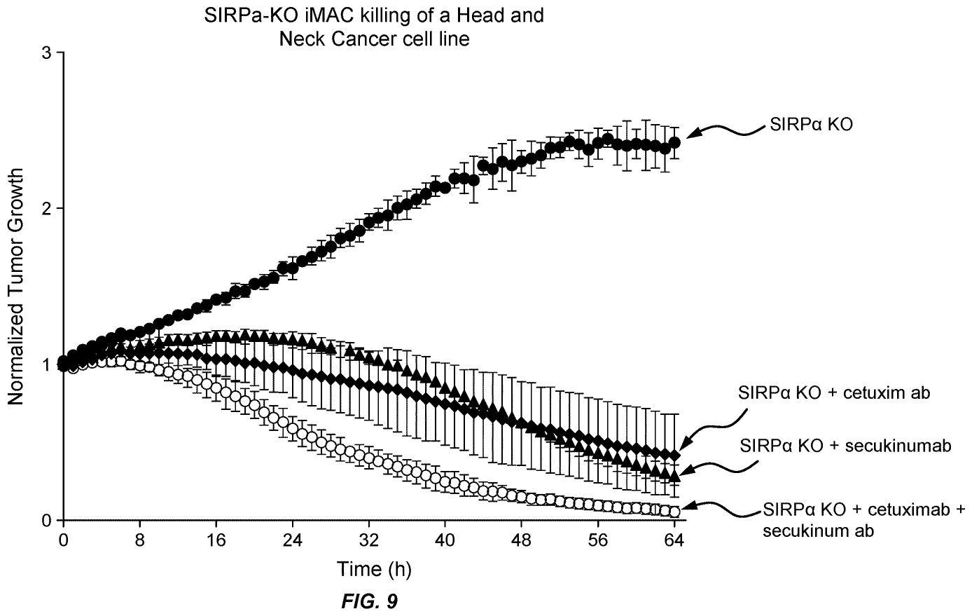

Briefly, SIRPA KO macrophages (“SIRPα” or “SIRPα KO”) derived from SIRPA KO iPSCs or counterpart wild-type control macrophages derived from the TC-1133 iPSC-cell line (“TC1133”) that were not knocked out for SIRPA were combined with CAL27 cells, which were head and neck cancer cells (EGFR⁺ TROP2⁺CD47⁺), at a 5:1 effector cell to target cell ratio. 10 μg/mL cetuximab (anti-EGFR antibody), 10 μg/mL secukinumab (anti-TROP2 antibody), or both was added to the culture. The amount of target cell survival was measured over time by eSIGHT (Agilent). The results demonstrated that SIRPA KO macrophages derived from SIRPA KO iPSCs had increased killing of CAL27 cells by targeting EGFR or TROP2 as compared to SIRPA KO macrophages not targeting any antigen (FIG. 9). In addition, the killing of CAL27 cells by SIRPA KO macrophages was further enhanced by targeting both EGFR and TROP2 (FIG. 9).

### 7.1.6. Example 6: CAR Targeting and Disruption of CD47-Sirpα Axis Synergized to Potently Kill Tumor Cells

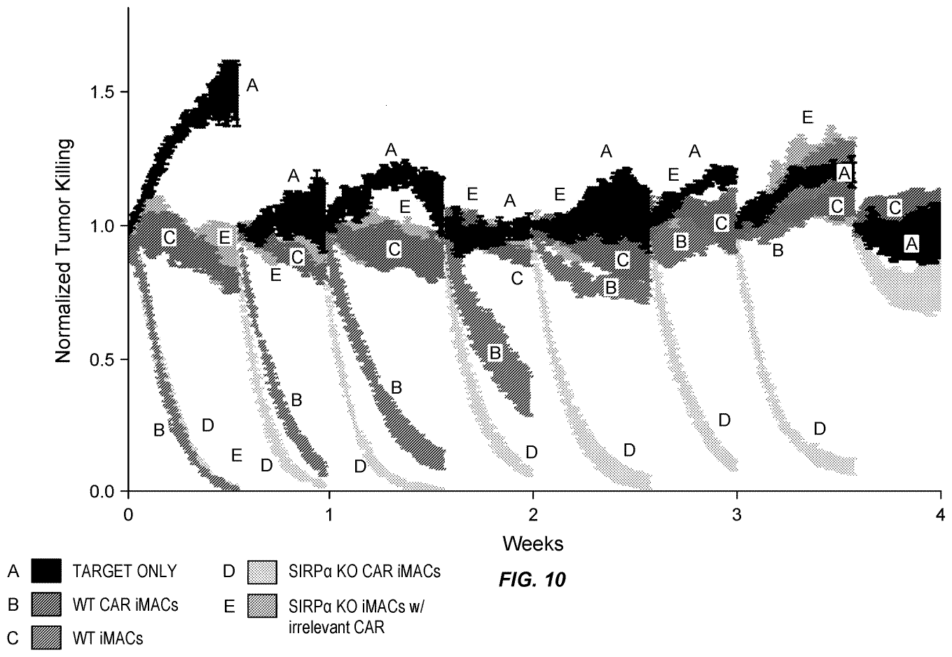

A CAR targeting PSMA (“PSMA-CAR) with a CD8 transmembrane and CD35 signaling domain design was generated and knocked in to SIRPA KO iPSCs or wildtype TC-1133 iPSC-cell line (“TC-113”) at the AAVS1 safe harbor location. CAR positive (CAR⁺) iPSCs were enriched by flow cytometry and then differentiated into SIRPA KO macrophages (“SIRPα” or “SIRPα KO”) or wildtype control macrophages (“WT”) derived from TC-1133 iPSC cell line that were not knocked out for SIRPA. The CAR macrophages were plated onto an eSIGHT assay plate and PSMA⁺ LNCAP target cells were added at an E:T ratio of 10:1. Every 4-6 days, fresh target cells were added and the normalized target cell killing was measured by the eSIGHT machine. WT and SIRPα KO PSMA-CAR⁺ macrophages were compared to target cells alone or an irrelevant CAR against a target not expressed on LNCAP cells (CLDN18.2). Over time, the SIRPα KO PSMA-CAR⁺ macrophages had better target cell killing following multiple target rechallenges than the WT PSMA-CAR⁺ macrophages (FIG. 10). This result demonstrated that disruption of CD47-SIRPα axis and CAR targeting synergized to potently kill tumor cells.

The nucleotide sequence of the polynucleotide encoding the PSMA-CAR used in Example 6 is as follows:

[SEQ ID NO: 61]
atggcactgccagtgaccgccctgctgctgccactggccctgctgct
gcacgcagccagaccccaagtgcaactggtggaatcaggcggcggag
tggtgcagcctggcagaagcctgcggctgtcctgcgcggcctctgga
ttcacattcagcagctacgccatgcactgggtcagacaggcccctgg
aaaaggcctggaatgggtggccgtgatcagctacgacggcaacaaca
agtactacgccgacagcgtgaagggaagattcaccatcagccgggac
aacagcaagaacaccctgtacctgcagatgaacagcctgcgggccga
ggatacagccgtctactattgtgccagagccgttccttggggctcca
gatactactactacggcatggacgtgtggggccagggcacaactgtg
accgtgtcctccggtggcggcggctccggcggaggcggaagcggagg
cggcggctctgctatccagctgacccagagcccatccagcctgtccg
ccagcgtgggcgatagagtgacaatcacctgtagagccagccaggga
atcagcagcgccctggcctggtatcagcaaaagagcggaaaagcccc
caagctgctgatcttcgacgcctctagcctggaatctggcgtgccta
gcagattcagcggatctggatctggcaccgactttaccctgacaatc
tctagcctccagccagaagatttcgctacatactactgccagcagtt
caacagctatcctctgaccttcggcggcggaaccaaagtggaaatca
agaccacaaccccagcacctagaccacccacaccagcacccaccatc
gcctctcagcctctgagcctgagaccagaggcctgtaggcccgcagc
aggaggagcagtgcacaccaggggcctggacttcgcctgcgacatct
acatctgggcacctctggccggcacctgtggcgtgctgctgctgagc
ctggtcatcacacgcgtgaagtttagccggtccgccgacgcaccagc
atatcagcagggccagaatcagctgtacaacgagctgaatctgggcc
ggagagaggagtacgacgtgctggataagaggcggggccgggacccc
gagatgggaggcaagccacagaggagaaagaacccccaggagggcct
gtataatgagctgcagaaggacaagatggccgaggcctactctgaga
tcggcatgaagggagagaggcgccggggcaagggacacgatggcctg
tatcagggcctgagcacagccaccaaggacacctacgatgccctgca
catgcaggccctgcctccaagg

The amino acid sequence of the PSMA-CAR used in Example 6 is as follows:

[SEQ ID NO: 62]
MALPVTALLLPLALLLHAARPQVQLVESGGGVVQPGRSLRLSCAASG
FTFSSYAMHWVRQAPGKGLEWVAVISYDGNNKYYADSVKGRFTISRD
NSKNTLYLQMNSLRAEDTAVYYCARAVPWGSRYYYYGMDVWGQGTTV
TVSSGGGGSGGGGSGGGGSAIQLTQSPSSLSASVGDRVTITCRASQG
ISSALAWYQQKSGKAPKLLIFDASSLESGVPSRFSGSGSGTDFTLTI
SSLQPEDFATYYCQQFNSYPLTFGGGTKVEIKTTTPAPRPPTPAPTI
ASQPLSLRPEACRPAAGGAVHTRGLDFACDIYIWAPLAGTCGVLLLS
LVITRVKFSRSADAPAYQQGQNQLYNELNLGRREEYDVLDKRRGRDP
EMGGKPQRRKNPQEGLYNELQKDKMAEAYSEIGMKGERRRGKGHDGL
YQGLSTATKDTYDALHMQALPPR

### 7.1.7. Example 7: SIRPA KO Macrophages had Increased Anti-Tumor Effects In Vivo

This example demonstrated that SIRPA KO macrophages had increased anti-tumor effects in vivo relative to macrophages expressing SIRPA.

Briefly, SIRPA KO macrophages (“SIRPA KO iMAC”) derived from SIRPA KO iPSCs or counterpart wild-type control macrophages derived from the TC-1133 iPSC-cell line (“WT iMAC”) were generated as described in Example 1. The SIRPA KO macrophages and wild-type control macrophages were precomplexed with rituximab prior to administration (“WT iMAC/RTX” or “SIRPA KO iMAC/RTX”). The predicted rituximab injected in precomplexed samples was 1 μg. Mice were injected by tail vein with 5×10⁵ Raji cells on day 0.

Four experimental groups, with 5 mice in each group, were treated as follows:
Group 1 (Vehicle Control): saline vehicle was injected intravenously to the mice on day 1 and day 4.
Group 2 (WT iMAC/RTX): wild-type control macrophage precomplexed with rituximab were injected intravenously to the mice on day 1 and day 4 at a dose of 1×10⁷ cells/mouse.
Group 3 (WT iMAC/RTX+aCD47): wild-type control macrophage precomplexed with rituximab were injected intravenously to the mice on day 1 and day 4 at a dose of 1×10⁷ cells/mouse. An anti-CD47 antibody was injected intraperitoneally to the mice at a dose of 250 μg/mouse every other day for four weeks.
Group 4 (SIRPA KO iMAC/RTX): SIRPA KO macrophages precomplexed with rituximab were injected intravenously to the mice on day 1 and day 4 at a dose of 1×10⁷ cells/mouse.

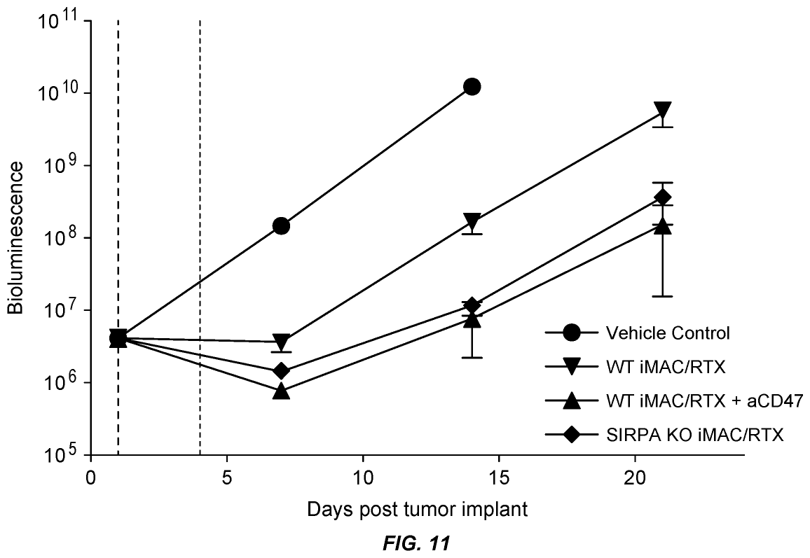

Weekly in vivo bioluminescence imaging was performed to monitor the tumor growth. As shown in FIG. 11, SIRPA KO macrophages had increased inhibition of tumor growth in vivo as compared to the counterpart wildtype-control macrophages.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A modified pluripotent cell comprising a polynucleotide encoding a chimeric antigen receptor (CAR) and genetic disruption of:
(a) a signal regulatory protein alpha (SIRPA) gene; and/or
(b) a sialic acid-binding Ig-like lectin 10 (SIGLEC10) gene.

2. The modified pluripotent cell of claim 1, wherein the pluripotent cell comprises genetic disruption of the SIRPA gene.

3. The modified pluripotent cell of claim 2, wherein the cell is heterozygous for the genetic disruption of the SIRPA gene.

4. The modified pluripotent cell of claim 2, wherein the cell is homozygous for the genetic disruption of the SIRPA gene.

5. The modified pluripotent cell of any one of claims 1 to 4, wherein the pluripotent cell comprises genetic disruption of the SIGLEC10 gene.

6. The modified pluripotent cell of claim 5, wherein the cell is heterozygous for the genetic disruption of the SIGLEC10 gene.

7. The modified pluripotent cell of claim 5, wherein the cell is homozygous for the genetic disruption of the SIGLEC10 gene.

8. The modified pluripotent cell of any one of claims 1 to 7, wherein the pluripotent cell further comprises genetic disruption of a CISH gene.

9. The modified pluripotent cell of claim 8, wherein the cell is heterozygous for the genetic disruption of a cytokine inducible SH2 containing protein (CISH) gene.

10. The modified pluripotent cell of claim 8, wherein the cell is homozygous for the genetic disruption of the CISH gene.

11. The modified pluripotent cell of any one of claims 1 to 10, wherein the CAR comprises a non-lymphoid intracellular signaling domain.

12. The modified pluripotent cell of claim 11, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

13. The modified pluripotent cell of any one of claims 1 to 12, wherein the modified pluripotent cell is an induced pluripotent stem cell (iPSC).

14. The modified pluripotent cell of claim 13, wherein the iPSC has been reprogrammed from a cell selected from the group consisting of a peripheral blood mononuclear cell (PBMC), CD34⁺ cord blood, a macrophage, a monocyte, and a fibroblast.

15. A method of generating a modified pluripotent cell, comprising:
(a) obtaining a pluripotent cell expressing a polypeptide encoding a chimeric antigen receptor (CAR); and
(b) genetically disrupting in the pluripotent cell:
(i) a signal regulatory protein alpha (SIRPA) gene; and/or
(ii) a sialic acid-binding Ig-like lectin 10 (SIGLEC10) gene.

16. A method of generating a modified pluripotent cell, comprising introducing a polypeptide encoding a chimeric antigen receptor (CAR) into a pluripotent cell comprising genetic disruption of:
(a) a signal regulatory protein alpha (SIRPA) gene; and/or
(b) a sialic acid-binding Ig-like lectin 10 (SIGLEC10) gene.

17. A method of generating a modified pluripotent cell, comprising:
(a) introducing a polypeptide encoding a chimeric antigen receptor (CAR) into a pluripotent cell; and
(b) genetically disrupting in the pluripotent cell:
(i) a signal regulatory protein alpha (SIRPA) gene; and/or
(ii) a sialic acid-binding Ig-like lectin 10 (SIGLEC10) gene.

18. A method of generating a modified pluripotent cell, comprising:
(a) genetically disrupting in a pluripotent cell:
(i) a signal regulatory protein alpha (SIRPA) gene; and/or
(ii) a sialic acid-binding Ig-like lectin 10 (SIGLEC10) gene; and
(b) introducing a polypeptide encoding a chimeric antigen receptor (CAR) into the pluripotent cell.

19. The method of any one of claims 15 to 18, wherein the method comprises genetically disrupting the SIRPA gene.

20. The method of claim 19, wherein the modified pluripotent cell is heterozygous for the genetic disruption of the SIRPA gene.

21. The method of claim 19, wherein the modified pluripotent cell is homozygous for the genetic disruption of the SIRPA gene.

22. The method of any one of claims 15 to 21, wherein the method comprises genetically disrupting the SIGLEC10 gene.

23. The method of claim 22, wherein the modified pluripotent cell is heterozygous for the genetic disruption of the SIGLEC10 gene.

24. The method of claim 22, wherein the modified pluripotent cell is homozygous for the genetic disruption of the SIGLEC10 gene.

25. The method of any one of claims 15 to 24, wherein the method further comprises genetically disrupting a CISH gene.

26. The method of claim 25, wherein the modified pluripotent cell is heterozygous for the genetic disruption of the CISH gene.

27. The method of claim 25, wherein the modified pluripotent cell is homozygous for the genetic disruption of the CISH gene.

28. The method of any one of claims 15 to 27, wherein the CAR comprises a non-lymphoid intracellular signaling domain.

29. The method of claim 28, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

30. The method of any one of claims 15 to 29, wherein the pluripotent cell is an induced pluripotent stem cell (iPSC).

31. The method of claim 30, wherein the iPSC has been reprogrammed from a cell selected from the group consisting of a peripheral blood mononuclear cell (PBMC), CD34⁺ cord blood, a macrophage, a monocyte, and a fibroblast.

32. A homogenous population of modified myeloid progenitor cells comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.

33. The population of modified myeloid progenitor cells of claim 32, wherein the cells comprise genetic disruption of the SIRPA gene.

34. The population of modified myeloid progenitor cells of claim 33, wherein the cells are heterozygous for the genetic disruption of the SIRPA gene.

35. The population of modified myeloid progenitor cells of claim 33, wherein the cells are homozygous for the genetic disruption of the SIRPA gene.

36. The population of modified myeloid progenitor cells of any one of claims 32 to 35, wherein the cells comprise genetic disruption of the SIGLEC10 gene.

37. The population of modified myeloid progenitor cells of claim 36, wherein the cells are heterozygous for the genetic disruption of the SIGLEC10 gene.

38. The population of modified myeloid progenitor cells of claim 36, wherein the cells are homozygous for the genetic disruption of the SIGLEC10 gene.

39. The population of modified myeloid progenitor cells of any one of claims 32 to 38, wherein the cells further comprise genetic disruption of a CISH gene.

40. The population of modified myeloid progenitor cells of claim 39, wherein the cells are heterozygous for the genetic disruption of the CISH gene.

41. The population of modified myeloid progenitor cells of claim 39, wherein the cells are homozygous for the genetic disruption of the CISH gene.

42. The population of modified myeloid progenitor cells of any one of claims 32 to 41, wherein the cells are isogenic.

43. The population of modified myeloid progenitor cells of any one of claims 32 to 42, wherein the CAR comprises a non-lymphoid intracellular signaling domain.

44. The population of modified myeloid progenitor cells of claim 43, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

45. The population of modified myeloid progenitor cells of any one of claims 32 to 44, wherein the cells are derived from induced pluripotent stem cells (iPSCs).

46. The population of modified myeloid progenitor cells of claim 45, wherein the iPSCs have been reprogrammed from a cell selected from the group consisting of a peripheral blood mononuclear cell (PBMC), CD34⁺ cord blood, a macrophage, a monocyte, and a fibroblast.

47. A method of generating the homogeneous population of modified myeloid progenitor cells of any one of claims 32 to 46, comprising expanding and differentiating the modified pluripotent cell of any one of claims 1 to 14 under conditions sufficient for cell differentiation into a population of myeloid progenitor cells.

48. The method of claim 47, wherein the modified pluripotent cell is an induced pluripotent stem cell (iPSC).

49. A homogenous population of modified monocytes comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.

50. The population of modified monocytes of claim 49, wherein the modified monocytes comprise genetic disruption of the SIRPA gene.

51. The population of modified monocytes of claim 50, wherein the modified monocytes are heterozygous for the genetic disruption of the SIRPA gene.

52. The population of modified monocytes of claim 50, wherein the modified monocytes are homozygous for the genetic disruption of the SIRPA gene.

53. The population of modified monocytes of any one of claims 49 to 52, wherein the modified monocytes comprise genetic disruption of the SIGLEC10 gene.

54. The population of modified monocytes of claim 53, wherein the modified monocytes are heterozygous for the genetic disruption of the SIGLEC10 gene.

55. The population of modified monocytes of claim 53, wherein the modified monocytes are homozygous for the genetic disruption of the SIGLEC10 gene.

56. The population of modified monocytes of any one of claims 49 to 55, wherein the modified monocytes further comprise genetic disruption of a CISH gene.

57. The population of modified monocytes of claim 56, wherein the modified monocytes are homozygous for the genetic disruption of the CISH gene.

58. The population of modified monocytes of claim 56, wherein the modified monocytes are homozygous for the genetic disruption of the CISH gene.

59. The population of modified monocytes of any one of claims 49 to 58, wherein the modified monocytes are isogenic.

60. The population of modified monocytes of any one of claims 49 to 59, wherein the CAR comprises a non-lymphoid intracellular signaling domain.

61. The population of modified macrophages of claim 60, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

62. The population of modified monocytes of any one of claims 49 to 61, wherein the modified monocytes are derived from induced pluripotent stem cells (iPSCs).

63. The population of modified monocytes of claim 62, wherein the iPSCs have been reprogrammed from a cell selected from the group consisting of a peripheral blood mononuclear cell (PBMC), CD34⁺ cord blood, a macrophage, a monocyte, and a fibroblast.

64. A method of generating the homogeneous population of monocytes of any one of claims 49 to 63, comprising expanding and differentiating the modified pluripotent cell of any one of claims 1 to 14 under conditions sufficient for cell differentiation into a population of monocytes.

65. The method of claim 64, wherein the modified pluripotent cell is an induced pluripotent stem cell (iPSC).

66. A homogenous population of modified macrophages comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.

67. The population of modified macrophages of claim 66, wherein the modified macrophages comprise genetic disruption of the SIRPA gene.

68. The population of modified macrophages of claim 67, wherein the modified macrophages are heterozygous for the genetic disruption of the SIRPA gene.

69. The population of modified macrophages of claim 67, wherein the modified macrophages are homozygous for the genetic disruption of the SIRPA gene.

70. The population of modified macrophages of any one of claims 66 to 69, wherein the modified macrophages comprise genetic disruption of the SIGLEC10 gene.

71. The population of modified macrophages of claim 70, wherein the modified macrophages are heterozygous for the genetic disruption of the SIGLEC10 gene.

72. The population of modified macrophages of claim 70, wherein the modified macrophages are homozygous for the genetic disruption of the SIGLEC10 gene.

73. The population of modified macrophages of any one of claims 66 to 72, the modified macrophages further comprise genetic disruption of a CISH gene.

74. The population of modified macrophages of claim 73, wherein the modified macrophages are heterozygous for the genetic disruption of the CISH gene.

75. The population of modified macrophages of claim 73, wherein the modified macrophages are homozygous for the genetic disruption of the CISH gene.

76. The population of modified macrophages of any one of claims 66 to 75, wherein the modified macrophages are isogenic.

77. The population of modified macrophages of any one of claims 66 to 76, wherein the CAR comprises a non-lymphoid intracellular signaling domain.

78. The population of modified macrophages of claim 77, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

79. The population of modified macrophages of any one of claims 66 to 78, wherein the modified macrophages are derived from induced pluripotent stem cells (iPSCs).

80. The population of modified macrophages of claim 79, wherein the iPSCs have been reprogrammed from a cell selected from the group consisting of a peripheral blood mononuclear cell (PBMC), CD34⁺ cord blood, a macrophage, a monocyte, and a fibroblast.

81. A method of generating the homogeneous population of macrophages of any one of claims 66 to 80 comprising expanding and differentiating the modified pluripotent cell of any one of claims 1 to 14 or the modified monocytes of any one of claims 49 to 63 under conditions sufficient for cell differentiation into a population of macrophages.

82. The method of claim 81, wherein the modified pluripotent cell is an induced pluripotent stem cell (iPSC).

83. A homogenous population of modified CD11b⁺CD45⁺ cells comprising a polynucleotide encoding a chimeric antigen receptor (CAR), and genetic disruption of:
(a) a signal regulatory protein alpha (SIRPA) gene; and/or
(b) a sialic acid-binding Ig-like lectin 10 (SIGLEC10) gene.

84. The population of modified CD11b⁺CD45⁺ cells of claim 83, wherein the cells comprise genetic disruption of the SIRPA gene.

85. The population of modified CD11b⁺CD45⁺ cells of claim 84, wherein the cells are heterozygous for the genetic disruption of the SIRPA gene.

86. The population of modified CD11b⁺CD45⁺ cells of claim 84, wherein the cells are homozygous for the genetic disruption of the SIRPA gene.

87. The population of modified CD11b⁺CD45⁺ cells of any one of claims 83 to 86, wherein the cells comprise genetic disruption of the SIGLEC10 gene.

88. The population of modified CD11b⁺CD45⁺ cells of claim 87, wherein the cells are heterozygous for the genetic disruption of the SIGLEC10 gene.

89. The population of modified CD11b⁺CD45⁺ cells of claim 87, wherein the cells are homozygous for the genetic disruption of the SIGLEC10 gene.

90. The population of modified CD11b⁺CD45⁺ cells of any one of claims 83 to 89, wherein the cells further comprise genetic disruption of a CISH gene.

91. The population of modified CD11b⁺CD45⁺ cells of claim 90, wherein the cells are heterozygous for the genetic disruption of the CISH gene.

92. The population of modified CD11b⁺CD45⁺ cells of claim 90, wherein the cells are homozygous for the genetic disruption of the CISH gene.

93. The population of modified CD11b⁺CD45⁺ cells of any one of claims 83 to 92, wherein the cells are isogenic.

94. The population of modified CD11b⁺CD45⁺ cells of any one of claims 83 to 93, wherein the CAR comprises a non-lymphoid intracellular signaling domain.

95. The population of modified CD11b⁺CD45⁺ cells of claim 94, wherein the non-lymphoid intracellular signaling domain is selected from the group consisting of BAI-1, CD86/B7-2, Lox1c, TM4, MEGF10, SCARF1, CD93, DAP12, SLAMF7, IFNγR2, 2B4/CD244, DECTIN-1, CD206, DECTIN-3, CLEC2, CD40, and CD80/B7-1.

96. The population of modified CD11b⁺CD45⁺ cells of any one of claims 83 to 95, wherein the cells are derived from induced pluripotent stem cells (iPSCs).

97. The population of modified CD11b⁺CD45⁺ cells of claim 96, wherein the iPSCs have been reprogrammed from a cell selected from the group consisting of a peripheral blood mononuclear cell (PBMC), CD34⁺ cord blood, a macrophage, a monocyte, and a fibroblast.

98. The population of modified CD11b⁺CD45⁺ cells of any one of claims 83 to 97, wherein the modified CD11b⁺CD45⁺ cell is a CD11b⁺CD45⁺CD14⁻ cell or a CD11b⁺CD45⁺CD14⁺ cell.

99. A method of generating the homogeneous population of modified CD11b⁺CD45⁺ cells of any one of claims 83 to 98, comprising expanding and differentiating the modified pluripotent cell of any one of claims 1 to 14 under conditions sufficient for cell differentiation into a population of CD11b⁺CD45⁺ cells.

100. The method of claim 99, wherein the modified pluripotent cell is an induced pluripotent stem cell (iPSC).
